(19) United States
(12) Patent Application Publication
(10) Pub. No.: US 2026/0226031 A1
(43) Pub. Date: Aug. 6, 2026

(54) NOVEL COMPOUNDS

(71) Applicant: NRG THERAPEUTICS LTD., Herts (GB)

(72) Inventors: Neil MILLER, Herts (GB); Gilles OUVRY, Herts (GB); Richard RUTTER, Herts (GB); Tammy LADDUWAHETTY, Cardiff (GB); Christopher THOMSON, Cardiff (GB); Jan KULAGOWSKI, London (GB); Michael ROWLEY, Essex (GB); Susan BOYD, Cambridge (GB)

(73) Assignee: NRG THERAPEUTICS LTD., Herts (GB)

(21) Appl. No.: 19/148,178

(22) PCT Filed: Jan. 19, 2024
(86) PCT No.: PCT/GB2024/050145

§ 371 Date: Jul. 15, 2025

(30) Foreign Application Priority Data
Jan. 19, 2023 (GB) 2300833.7

(51) Int. Cl. C07D 403/06; A61K 31/4192; A61K 31/4196; A61K 31/422; A61K 31/4245; A61K 31/4439; A61K 31/454; A61K 31/497; A61K 31/513; C07D 401/06; C07D 413/06

(52) U.S. Cl. CPC C07D 403/06; A61K 31/4192; A61K 31/4196; A61K 31/422; A61K 31/4245; A61K 31/4439; A61K 31/454; A61K 31/497; A61K 31/513; C07D 401/06; C07D 413/06

(57) ABSTRACT

The invention relates to compounds of formula (I), and related aspects.

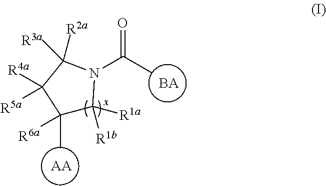

### FIELD OF THE INVENTION

The invention relates to novel compounds which are inhibitors of the mitochondrial permeability transition pore (mPTP). The invention also inter alia relates to such compounds for use as medicaments, in particular, for the treatment or prevention of degenerative, neurodegenerative or mitochondrial diseases or other diseases or disorders in which inhibition of mPTP provides a therapeutic or prophylactic effect.

### BACKGROUND TO THE INVENTION

The mitochondria permeability transition pore (mPTP) is a high conductance channel residing on the inner mitochondrial membrane that is activated under certain conditions of cellular stress, in particular excessive Ca²⁺ loading and oxidative stress. It is permeable to solutes with molecular mass <1.5 kDa, is voltage and Ca²⁺ dependent and exhibits a characteristic large conductance. Once activated, oxidative phosphorylation is uncoupled resulting in the loss of the mitochondria membrane potential and disrupted mitochondria metabolism. In addition, solutes enter the mitochondrial matrix resulting in swelling, eventual rupture of the outer membrane with consequent release of apoptotic factors as well as sequestered Ca²⁺, leading to eventual cell death via apoptosis or necrosis depending on the type and physiology of the cell. As such it has been implicated as a key pathological event in multiple degenerative and metabolic diseases.

Under normal physiological conditions mitochondria play a key role in regulating cellular Ca²⁺ homeostasis. Ca²⁺ entering the cell via cell surface channels, a common mechanism of cell signalling, is rapidly sequestered by mitochondria, preventing excessive and toxic Ca²⁺ accumulation in the cell cytoplasm. In cell types such as neurons, skeletal muscle myofibers and cardiomyocytes which undergo high levels of Ca²⁺ flux, this Ca²⁺‘buffering’ effect of mitochondria is critical to maintain cell health. However, there is a limit to the capacity of mitochondria to sequester Ca²⁺ and if intramitochondrial Ca²⁺ levels reach a certain threshold the Ca²⁺ sensitive mPTP is activated, resulting in collapse of the mitochondria and initiation of cell death. Activation of the mPTP in degenerative diseases may occur in a variety of ways depending on the disease, for example: 1) excessive Ca²⁺ entry into cells and overload of the mitochondria with Ca²⁺2) dysfunctional mitochondrial Ca²⁺ efflux mechanisms, in particular decreased activity of the Ca²⁺ efflux transporter NCLX resulting in Ca²⁺ overload 3) overactivity or upregulation of the Ca²⁺ uptake mechanisms in mitochondria 4) oxidative stress 5) sensitization of the mPTP due to compromised mitochondrial function i.e. mPTP activation at lower intramitochondrial concentrations of Ca²⁺6) excessive transfer of Ca²⁺ from the endoplasmic reticulum into the mitochondria at contact points between the two organelles known as mitochondria-associated-membranes.

While the properties and function of the mPTP can be studied in simple in vitro assays in isolated mitochondria, the molecular identity of the mPTP is not known. Multiple proteins have been proposed to comprise the pore forming complex, including the ATP synthase and the adenine nucleotide transporter (ANT) family of proteins but no single protein is widely accepted as being responsible for formation of the pore. However, the peptidyl prolyl cis-trans isomerase F (Ppif—Uniprot ID P30405 and also known as cyclophilin D), is well accepted to be a key regulator of the pore, though not forming a transmembrane channel in its own right. Genetic or pharmacological inhibition of Ppif significantly decreases the sensitivity of pore opening in response to Ca²⁺ loading and other mPTP activators. Genetic ablation or pharmacological inhibition of Ppif has therefore been utilised to evaluate involvement of the mPTP in pathological pathways in cell and animal disease models. In this way, inhibition of the mPTP has been shown to be protective in numerous models of disease, in particular those where Ca²⁺ dysregulation and oxidative stress are known to contribute to cellular degeneration. Notably, genetic knockout of Ppif was shown to be protective in various preclinical in vivo transgenic models of neurodegenerative disease including Alzheimer's disease, Parkinson's disease and motor neuron disease, also known as Amyotrophic Lateral Sclerosis (ALS), demonstrating the therapeutic potential of mPTP inhibition. In each of these diseases, genetic mutations in particular proteins that cause inherited forms of disease (i.e. amyloid precursor protein, alpha-synuclein and superoxide dismutase 1 respectively), and are expressed in the mouse models, have been shown to cause either Ca²⁺ overload of the mitochondria or sensitization of the mPTP. Recent evidence suggests this may occur through a common mechanism in Alzheimer's, Parkinson's and Friedreich's ataxia. In each case, it has been reported that in cells expressing the mutated disease associated proteins (amyloid precursor protein, PINK1 and frataxin respectively), the activity or expression of the mitochondrial Ca²⁺ efflux transporter, NCLX, is decreased, resulting in Ca²⁺ overload of the mitochondria. In the case of Parkinson's disease, the pathological aggregated form of the protein alpha-synuclein, a common misfolded protein in sporadic and inherited cases of Parkinson's disease, has also been shown to sensitise and activate the mPTP.

Genetic ablation of Ppif has been shown to be beneficial in numerous other preclinical models of degenerative disease, therefore demonstrating the potential of mPTP inhibitors in Duchenne and congenital forms of muscular dystrophy, ischemia-reperfusion injury, bone repair, pancreatitis and inter alia other associated disorders.

In addition to the demonstrated benefit of Ppif inhibition in preclinical models, mPTP function has been shown to be dysregulated in multiple other disease indications. In particular, in a number of diseases the threshold for mPTP activation in response to Ca²⁺ loading appears to be sensitised suggesting that mPTP activation may occur aberrantly under physiological conditions and drive tissue degeneration. For example, in muscle mitochondria from elderly human muscle biopsies, the threshold for mPTP activation is reduced compared to healthy control. In these diseases, this sensitization of mPTP activity underlies additional rationale for the therapeutic potential of mPTP inhibitors.

mPTP inhibitors may also have therapeutic potential in other diseases where mitochondrial dysfunction, oxidative stress, inflammatory stress or Ca²+ dysregulation occur during disease pathogenesis.

The discovery and development of inhibitors of the mPTP has largely been focussed on identification of Ppif inhibitors. Cyclosporin A (CsA), originally identified as an immunosuppressant by virtue of its inhibitory activity at calcineurin, was also found to inhibit Ppif as well as other members of the peptidyl prolyl cis-trans isomerase (Ppi) enzyme family. Several cyclosporin A derivatives e.g. Debio-25, NIM811 were subsequently developed that retained broad activity against the Ppi enzyme family without inhibiting calcineurin, however none of these progressed to market. To date, no potent brain penetrant selective Ppif inhibitors have been reported. Other more recent approaches to discover mPTP inhibitors, have utilised phenotypic screens in isolated mitochondria. These have successfully identified potent small molecule mPTP inhibitors with a Ppif-independent mode of action.

Yu et al., (2020, Cell, 183, 1-14) relates to the link between mPTP activation and the mechanism of TDP-43 proteinopathy such as TAR DNA-binding protein 43 (TDP-43) associated neurodegeneration. Accumulation in neuronal cytoplasm of the normally nuclear protein TDP-43 is a disease hallmark for almost all cases of ALS and 40-50% of Frontotemporal Lobar Degeneration (FTLD), with some familial cases caused by mutant forms of the protein. Both diseases are associated with a neuroinflammatory cytokine profile related to upregulation of NF-κB and type I IFN pathways, directly suggesting a role for TDP-43 in neuroinflammation. Mutant or overexpressed WT TDP-43 in neurons mis-localises to the mitochondria and induces the release of mitochondrial DNA (mtDNA) into the cytoplasm. This mtDNA then activates the immune sensor cGAS-STING triggering the induction of innate immune genes such as IL-6, TNFα, and interferons. Inhibition of the mPTP with cyclosporin A or via Ppif knockout, prevents the TDP-43 induced release of mtDNA and subsequent induction of innate immune response genes. Furthermore, inhibition of cGAS-STING extends the survival of mutant mice expressing a mutant TDP-43. The data implicates mPTP activation in mediating the toxic effects of TDP-43 in ALS and other disease where either mutations in the TDP-43 gene causes disease or where TDP-43 proteinopathy is observed.

Jang et al., (2021 American Journal of Physiology: Renal physiology, 2021 321:4, F431-F442) highlighted the potential therapeutic benefit of mPTP inhibition (via Ppif knockout) in a mouse model of kidney fibrosis. Unilateral ureteral obstruction was used to induce kidney fibrosis in WT and Ppif KO mice. Inflammation, proximal tubule atrophy and markers of fibrosis were reduced in the Ppif KO mice versus WT. Measures of fibrosis included collagen deposition, α-SMA and TGFβ expression, and interstitial cell proliferation. This highlights the potential role of the mPTP in cell injury/death-mediated tissue remodelling and fibrogenesis. mPTP inhibitors may therefore be beneficial in diseases where fibrosis is a key pathological mechanism, e.g. chronic kidney disease, idiopathic pulmonary fibrosis, non-alcoholic steatohepatitis, primary biliary cholangitis and systemic sclerosis.

WO2010/049768 relates to acrylamido derivatives and their use as therapeutic agents, particularly for the prevention and/or treatment of diseases associated with the activity of the mPTP (see also Plyte et al. J. Med Chem. 2014, 57, 5333-47). Chen et al. (Assay and Drug Development Technologies, 2018, 16, 445-455) relates to phenotypic screening for mPTP modulators using platelets, and discloses further acrylamido derivatives. CA2884607A1 relates to acrylamido and maleimide compounds which are said to be useful in the treatment of mitochondrial diseases. WO2022/049376, WO2022/049377 and WO2023/166303 disclose cinnamide compounds which are inhibitors of the mPTP.

There remains a need to find further compounds which are inhibitors of the mPTP.

### SUMMARY OF THE INVENTION

In a first aspect, the invention provides a compound according to formula (I):

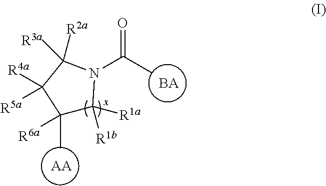

wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is H, C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the carbon atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl, wherein said phenyl or phenyl fused to C₅₋₆cycloalkyl may be optionally substituted by one or more AA¹;
   AA¹ is halo, C₂₋₆alkynyl, C₁₋₆alkyl C₁₋₆haloalkyl, C₁₋₆alkoxy, C₁₋₆haloalkoxy, C₃₋₆ cycloalkyl, CN, OH, NR^{q}R^{r}, or NHSO₂R^{t};
      R^{q} is H or C₁₋₄alkyl;
      R^{r} is H or C₁₋₄alkyl;
      R^{t} is C₁₋₄alkyl;
   BA is a monocyclic or bicyclic heterocycle or a monocyclic or bicyclic heteroaryl, wherein said heterocycle or heteroaryl may be optionally substituted by one or more B^{1A}; and
      B^{1A} is halo, C₁₋₆alkyl, C₁₋₆haloalkyl, oxo (=O), C₁₋₆alkoxy, C₁₋₆haloalkoxy, or C₀₋₆alkylene(OH);
         or a salt and/or solvate thereof,
         and provided that the compound of formula (I) is not 5-hydroxypyridin-3-yl)(8-phenyl-6-azaspiro[3.4]octan-6-yl)methanone:

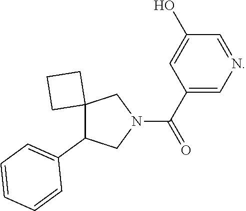 [SMILES: O=C(c1cncc(O)c1)N1CC(c2ccccc2)C2(CCC2)C1]

In one embodiment, a compound of formula (I) is provided in the form of a salt. In one embodiment, a compound of formula (I) is provided in the form of a pharmaceutically acceptable salt. In one embodiment, a compound of formula (I) is provided in the form of a solvate. In one embodiment, a compound of formula (I) is provided in the form of a pharmaceutically acceptable solvate. In one embodiment, a compound of formula (I) is provided in the form of a pharmaceutically acceptable salt and solvate (i.e. a pharmaceutically acceptable salt of a pharmaceutically acceptable solvate). In one embodiment, a compound of formula (I) is provided.

The invention further provides pharmaceutical compositions comprising a compound of formula (I), or a pharmaceutically acceptable salt and/or solvate thereof, and a pharmaceutically acceptable carrier or excipient, wherein the proviso of formula (I) does not apply.

The invention also provides the use of a compound of formula (I), or pharmaceutically acceptable salt and/or solvate thereof, in the manufacture of a medicament for the treatment or prophylaxis of a disease or disorder in which inhibition of mPTP provides a therapeutic or prophylactic effect, wherein the proviso of formula (I) does not apply.

The invention also provides the use of a compound of formula (I), or a pharmaceutically acceptable salt and/or solvate thereof, the manufacture of a medicament for the treatment or prophylaxis of a disease or disorder in which inhibition of mPTP provides a therapeutic or prophylactic effect, wherein the proviso of formula (I) does not apply.

The invention also provides a method of preventing or treating a disorder in which inhibition of mPTP provides a therapeutic or prophylactic effect in a subject, which comprises administering to a subject in need thereof an effective amount of a compound of formula (I), or a pharmaceutically acceptable salt and/or solvate thereof, wherein the proviso of formula (I) does not apply.

Suitably the disease or disorder is selected from degenerative or neurodegenerative diseases, disorders of the central nervous system, ischemia and re-perfusion injury, metabolic diseases, inflammatory or autoimmune diseases, diseases of aging and renal diseases.

The invention also provides a compound of formula (I), or a pharmaceutically acceptable salt and/or solvate thereof, for use in the treatment or prophylaxis of a mitochondrial disease, wherein the proviso of formula (I) does not apply.

The invention also provides the use of a compound of formula (I), or a pharmaceutically acceptable salt and/or solvate thereof, in the manufacture of a medicament for the treatment or prophylaxis of a mitochondrial disease, wherein the proviso of formula (I) does not apply.

The invention also provides a method of preventing or treating a mitochondrial disease in a subject, which comprises administering to a subject in need thereof an effective amount of a compound of formula (I), or a pharmaceutically acceptable salt and/or solvate thereof, wherein the proviso of formula (I) does not apply.

The invention also provides a compound of formula (I), or a pharmaceutically acceptable salt and/or solvate thereof, for use in the treatment or prophylaxis of a disease or disorder associated with TDP-43 proteinopathy such as TDP-43 associated neurodegeneration, wherein the proviso of formula (I) does not apply.

The invention also provides the use of a compound of formula (I), or a pharmaceutically acceptable salt and/or solvate thereof, in the manufacture of a medicament for the treatment or prophylaxis of a disease or disorder associated with TDP-43 proteinopathy such as TDP-43 associated neurodegeneration, wherein the proviso of formula (I) does not apply.

The invention also provides a method of treating or preventing a disease or disorder associated with TDP-43 proteinopathy such as TDP-43 associated neurodegeneration, which comprises administering to a subject in need thereof an effective amount of a compound of formula (I), or a pharmaceutically acceptable salt and/or solvate thereof, wherein the proviso of formula (I) does not apply.

The invention also provides a compound of formula (I), or a pharmaceutically acceptable salt and/or solvate thereof, for use in the treatment or prophylaxis of a disease or disorder associated with fibrosis, wherein the proviso of formula (I) does not apply.

The invention also provides the use of a compound of formula (I), or a pharmaceutically acceptable salt and/or solvate thereof, in the manufacture of a medicament for the treatment or prophylaxis of a disease or disorder associated with fibrosis, wherein the proviso of formula (I) does not apply.

The invention also provides a method of treating or preventing a disease or disorder associated with fibrosis, which comprises administering to a subject in need thereof an effective amount of a compound of formula (I), or a pharmaceutically acceptable salt and/or solvate thereof, wherein the proviso of formula (I) does not apply.

### DETAILED DESCRIPTION OF THE INVENTION

The term “alkyl” as used herein, such as in C₁₋₄alkyl, whether alone or forming part of a larger group, is a straight or branched fully saturated hydrocarbon chain containing the specified number of carbon atoms. Examples of C₁₋₄alkyl groups include methyl, ethyl, n-propyl, iso-propyl, n-butyl, iso-butyl, tert-butyl and sec-butyl. Reference to “propyl” includes n-propyl and iso-propyl. Reference to “butyl” includes n-butyl, iso-butyl, tert-butyl and sec-butyl.

The term “alkylene” as used herein, such as C₁₋₄alkylene or C₁₋₆alkylene, whether alone or forming part of a larger group e.g. C₁₋₄alkylene(OH), C₁₋₆alkylene(OH) or C₁₋₆alkylene(C₃₋₆ cycloalkyl), is a bifunctional straight or branched fully saturated hydrocarbon group containing the specified number of carbon atoms. Examples of C₁₋₆alkylene groups include methylene (i.e. —CH₂—), ethylene (i.e. —CH₂CH₂—) n-propylene (i.e. (—CH₂)₃—), n-butylene (i.e. (—CH₂)₄—), n-pentylene (i.e. (—CH₂)₅—) and n-hexylene (i.e. (—CH₂)₆—). A branched example of a C₁₋₆alkylene group is i-propylene (i.e. —CH(Me)CH₂—). References to C₀alkylene will be understood to mean that the alkylene chain is absent e.g. C₀alkylene(OH) represents OH.

The term C₁₋₄alkylene(OH) as used herein e.g. C₁₋₄alkylene means a C₁₋₄alkyl group substituted by OH, such as CH₂OH. References to C₀alkylene will be understood to mean that the alkylene chain is absent e.g. C₀alkylene(OH) represents OH.

The term “alkoxy” as used herein, such as in C₁₋₄alkoxy or C₁₋₆alkoxy, refers to an alkyl group (e.g. a C₁₋₄alkyl group) as defined above, singularly bonded to an oxygen atom. Examples of C₁₋₆alkoxy groups include methoxy, ethoxy, 1-propoxy, 2-propoxy, 1-butoxy, 2-butoxy and 3-butoxy, especially methoxy.

The term ‘halo’ or ‘halogen’ as used herein, refers to fluorine, chlorine, bromine or iodine. Particular examples of halo are bromine, fluorine and chlorine, especially fluorine.

The term “haloalkyl” as used herein, such as in C₁₋₄haloalkyl or C₁₋₆haloalkyl, is a straight or branched alkyl group containing the specified number of carbon atoms, substituted by one or more halo atoms, for example fluoromethyl (CH₂F), di-fluoromethyl (CHF₂), tri-fluoromethyl (CF₃), 1-fluoroethyl (CH₂FCH₂) and 2-fluoroethyl (CH₂CH₂F).

The term “cycloalkyl” as used herein, such as in C₃₋₆cycloalkyl, whether alone or forming part of a larger group such as C₀₋₆alkylene(C₃₋₆cycloalkyl) is a fully saturated hydrocarbon ring containing the specified number of carbon atoms. Examples of C₃₋₆cycloalkyl groups include cyclopropyl, cyclobutyl, cyclopentyl and cyclohexyl, in particular cyclopropyl. Optionally, the cycloalkyl may be substituted as defined herein.

The term ‘alkenyl’ as used herein, such as in C₂₋₆alkenyl, refers to a straight or branched hydrocarbon group containing the specified number of carbon atoms and at least one carbon-carbon double bond, such as one or two double bonds. The term encompasses, CH═CH₂, CH₂CH═CH₂, CH═CHCH₃, CH₂CH₂CH═CH₂, CH═CHCH₂CH₃, CH₂CH═CHCH₃, CH₂CH₂CH₂CH═CH₂, CH═CHCH₂CH₂CH₃, CH₂CH═CHCH₂CH₃, CH₂CH₂CH═CHCH₃, CH═CHCH═CHCH₃ and CH₂CH═CHCH═CH₂. Branched variants such as CH(CH₃)CH═CH₂ and CH═C(CH₃)₂ are also included.

The term ‘haloalkenyl’ as used herein, such as in C₂₋₆haloalkenyl is a straight or a branched alkenyl chain containing the specified number of carbon atoms and at least one halogen atom, such as fluoro or chloro, for example fluoro.

The term ‘aryl’ as used herein refers to a mono (i.e. phenyl) or polycyclic ring system (e.g. comprising one or two, such as one additional ring) containing at least one phenyl ring, suitably an aryl group contains 6-10 ring members. Additional rings in a polycylic ring system may be saturated (e.g forming indane or tetralin) or partially unsaturated (e.g. forming indene) or fully unsaturated (e.g. forming naphthalene) hydrocarbon rings, or additional rings may be saturated or partially unsaturated heterocycles (e.g. forming chromane). Suitably aryl refers to a mono (i.e. phenyl) or a bicyclic ring system containing at least one phenyl ring (and no heteroaryl rings).

The term ‘heteroaryl’ as used herein refers to mono or polycyclic ring system (e.g bicyclic) with at least one ring having aromatic character and containing at least one heteroatom selected from N, O and S, for example N. Suitably a heteroaryl group contains 5-10 ring members. Where a heteroaryl group contains more than one ring, not all rings must contain a heteroatom, and not all rings must be aromatic in character. In some examples heteroaryl is monocyclic, such as a 5- or 6-membered heteroaryl ring (e.g. containing one or two heteroatom selected from N, S and O). In other examples heteroaryl is bicyclic, such as a 5,5-, 5,6- or 6,6-bicyclic system (e.g. containing one, two or three heteroatoms selected from N, S and O). A heteroaryl may contain one heteroatom selected from N, S and O, for example N and O, especially N. In other examples a heteroaryl may contain two heteroatoms selected from N, S and O. In further examples a heteroaryl may contain three heteroatoms selected from N, S and O, for example N and O. Examples of 6-membered heteroaryls include one nitrogen atom (pyridinyl), two nitrogen atoms (pyridazinyl, pyrimidinyl or pyrazinyl) and three nitrogen atoms (triazinyl). Further examples of heteroaryl include triazolyl, indolyl, indazolyl, benzofuranyl, benzimidazolyl, benzoxazolinyl, quinolinyl, isoquinolinyl and quinazolinyl.

As used herein, the term heterocycle means a non-aromatic cyclic group of carbon atoms wherein from one to four of the carbon atoms is/are replaced by one or more heteroatoms independently selected from nitrogen (N), oxygen (O) or sulfur (S). A heterocycle group may, for example, be monocyclic or bicyclic. In a bicyclic heterocycle group there may be one or more heteroatoms in each ring, or only in one of the rings. Where a heterocycle group contains more than one ring, not all rings must contain a heteroatom. A heteroatom may be S, O or N, and is suitably O or N. Suitably a heterocycle group contains 5-10 ring members.

A heterocycle may contain one heteroatom selected from N, S and O, for example N and O, especially N. In other examples a heterocycle may contain two heteroatoms selected from N, S and O. In further examples a heterocycle may contain three heteroatoms selected from N, S and O, for example N and O. In some examples the heterocycle is monocyclic, such as a 5- or 6-membered heterocycle ring. Examples of heterocycles include morpholinyl, tetrahydrofuran, and tetrahydropyran.

Where substituents are indicated as being optionally substituted in formula (I) or (IB) in the embodiments and preferences set out below, the optional substituent may be attached to an available carbon atom, which means a carbon atom which is attached to a hydrogen atom i.e. a C—H group or the optional substituent may be attached to an available nitrogen atom, which means a nitrogen atom which is attached to a hydrogen atom i.e. a N—H group. The optional substituent replaces the hydrogen atom attached to the carbon atom or the hydrogen atom attached to the nitrogen atom.

The invention provides a compound according to formula (I):

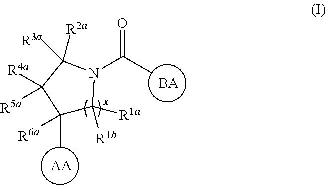

wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is H, C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the carbon atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl wherein said phenyl or phenyl fused to C₅₋₆cycloalkyl may be optionally substituted by one or more AA¹;
   AA¹ is halo, C₂₋₆alkynyl, C₁₋₆alkyl, C₁₋₆haloalkyl, C₁₋₆alkoxy, C₁₋₆haloalkoxy, C₃₋₆ cycloalkyl, CN, OH, NR^{q}R^{r}, NHSO₂R^{t};
      R^{q} is H or C₁₋₄alkyl;
      R^{r} is H or C₁₋₄alkyl;
      R^{t} is C₁₋₄alkyl;
   BA is a monocyclic or bicyclic heterocycle or a monocyclic or bicyclic heteroaryl wherein said heterocycle or heteroaryl may be optionally substituted by one or more B^{1A}; and
      B^{1A} is halo, C₁₋₆alkyl, C₁₋₆haloalkyl, oxo (=O), C₁₋₆alkoxy, C₁₋₆haloalkoxy, or C₀₋₆alkylene(OH);
         or a salt and/or solvate thereof,
         and provided that the compound of formula (I) is not 5-hydroxypyridin-3-yl)(8-phenyl-6-azaspiro[3.4]octan-6-yl)methanone:

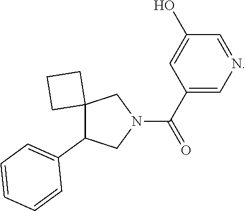 [SMILES: O=C(c1cncc(O)c1)N1CC(c2ccccc2)C2(CCC2)C1]

In an embodiment, the invention provides a compound according to formula (IB):

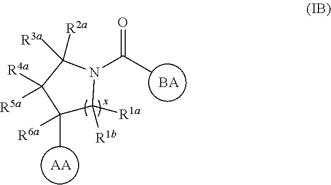

wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is H, C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl optionally substituted by one or more AA¹;
   AA¹ is halo, C₁₋₆alkyl C₁₋₆haloalkyl, C₁₋₆alkoxy, C₁₋₆haloalkoxy, C₃₋₆cycloalkyl, CN, OH, NR^{q}R^{r}, or NHSO₂R^{t};
      R^{q} is H or C₁₋₄alkyl;
      R^{r} is H or C₁₋₄alkyl;
      R^{t} is C₁₋₄alkyl;
   BA is a monocyclic or bicyclic heterocycle or a monocyclic or bicyclic heteroaryl which may be optionally substituted by one or more B^{1A}; and
      B^{1A} is halo, C₁₋₆alkyl, C₁₋₆haloalkyl, C₁₋₆alkoxy, C₁₋₆haloalkoxy, or C₀₋₆alkylene(OH);
         or a salt and/or solvate thereof,
         and provided that formula (IB) is not 5-hydroxypyridin-3-yl)(8-phenyl-6-azaspiro[3.4]octan-6-yl)methanone:

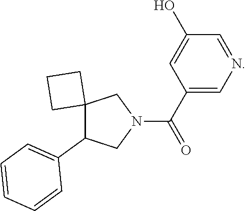 [SMILES: O=C(c1cncc(O)c1)N1CC(c2ccccc2)C2(CCC2)C1]

The compounds according to formula (IB) or a salt and/or solvate thereof are a sub-genus of compounds of formula (I) or a salt and/or solvate thereof. Compounds according to formula (IB′) or a salt and/or solvate thereof described below are a sub-genus of compounds of formula (IB) or a salt and/or solvate thereof. The embodiments that follow apply equally to the compounds of formula (I), (IB) and/or (IB′) or a salt and/or solvate thereof.

In one embodiment, R^{1a} is H. In a second embodiment, R^{1a} is C₁₋₄alkyl, such as methyl.

In one embodiment, R^{1b} is H. In a second embodiment, R^{1b} is C₁₋₄alkyl, such as methyl.

In one embodiment, R^{1a} and R^{1b} are each H.

In one embodiment, R^{2a} is H. In a second embodiment, R^{2a} is halo. In a third embodiment, R^{2a} is C₁₋₄alkyl. In a fourth embodiment, R^{2a} is C₁₋₄haloalkyl.

In one embodiment, R^{3a} is H. In a second embodiment, R^{3a} is halo. In a third embodiment, R^{3a} is C₁₋₄alkyl.

In one embodiment, R^{2a} and R^{3a} are each H.

In one embodiment R^{4a} is H. In a second embodiment, R^{4a} is C₁₋₄alkyl, such as methyl.

In one embodiment, R^{5a} is H. In a second embodiment, R^{5a} is C₁₋₆alkyl, such as methyl.

In a third embodiment, R^{5a} is C₁₋₆haloalkyl. In a fourth embodiment, R^{5a} is C₁₋₆alkoxy. In a fifth embodiment, R^{5a} is C₁₋₆haloalkoxy. In a sixth embodiment, R^{5a} is C₂₋₆alkenyl. In a seventh embodiment, R^{5a} is C₂₋₆haloalkenyl. In an eighth embodiment, R^{5a} is C₀₋₆alkylene(C₃₋₆cycloalkyl).

In a ninth embodiment, R^{5a} is C₀₋₆alkylene(OH).

In one embodiment, R^{4a} and R^{5a} are each methyl.

In one embodiment, R^{4a} and R^{5a} together with the atom to which they are attached form a C₃₋₆cycloalkyl. Within such embodiments, the C₃₋₆cycloalkyl may be substituted by one or more (such as one, two or three, e.g. one or two) groups selected from the group consisting of C₁₋₄alkyl, C₁₋₄haloalkyl and halo. Suitably, the C₃₋₆cycloalkyl is substituted by one group selected from the group consisting of C₁₋₄alkyl, C₁₋₄haloalkyl and halo. Suitably, the C₃₋₆cycloalkyl is not substituted. In one embodiment, R^{4a} and R^{5a} together with the atom to which they are attached form a cyclobutyl.

In one embodiment, R^{6a} is H. In a second embodiment, R^{6a} is halo. In a third embodiment, R^{6a} is C₁₋₄alkyl. In a fourth embodiment, R^{6a} is C₁₋₄haloalkyl. In a fifth embodiment, R^{6a} is C₁₋₄alkoxy. In a sixth embodiment, R^{6a} is C₁₋₄haloalkoxy.

In one embodiment, x is 0. In a second embodiment, x is 1. In a third embodiment, x is 2.

In one embodiment, AA is phenyl. In a second embodiment, AA is phenyl fused to C₅₋₆ cycloalkyl.

In one embodiment, AA is substituted by one or more (such as one, two or three, for example one or two, particularly one) AA¹.

In one embodiment, at least one AA¹ is halo, such as F. In a second embodiment, at least one AA¹ is C₁₋₆alkyl. In a third embodiment, at least one AA¹ is C₁₋₆haloalkyl. In a fourth embodiment, at least one AA¹ is C₁₋₆alkoxy. In a fifth embodiment, at least one AA¹ is C₅₋₆ haloalkoxy. In a sixth embodiment, at least one AA¹ is C₃₋₆cycloalkyl. In a seventh embodiment, at least one AA¹ is CN. In an eighth embodiment, at least one AA¹ is OH. In a ninth embodiment, at least one AA¹ is C₂₋₆alkynyl. In a tenth embodiment, at least one AA¹ is NR^{q}R^{r} Suitably, R^{q} is H. Suitably, R^{q} is C₁₋₄alkyl. Suitably, R^{r} is H. Suitably, R^{r} is C₁₋₄alkyl. In a eleventh embodiment, at least one AA¹ is NHSO₂R^{t}.

In one embodiment, AA is substituted by one, two or three AA¹, wherein the AA¹ is halo.

In one embodiment, AA is not substituted.

In one embodiment, BA is a monocyclic heterocycle. Suitably, BA is a 5-membered monocyclic heterocycle. In a second embodiment, BA is a bicyclic heterocycle. In a third embodiment, BA is a monocyclic heteroaryl. Suitably, BA is a 6-membered monocyclic heteroaryl. In a fourth embodiment, BA is a bicyclic heteroaryl.

In one embodiment, BA is substituted by one or more (such as one, two or three, for example one or two, particularly one) B^{1A}.

In one embodiment, at least one B^{1A} is halo. In a second embodiment, at least one B^{1A} is C₁₋₆alkyl, such as methyl. In a third embodiment, at least one B^{1A} is C₁₋₆haloalkyl. In a fourth embodiment, at least one B^{1A} is C₁₋₆alkoxy. In a fifth embodiment, at least one B^{1A} is C₁₋₆haloalkoxy. In a sixth embodiment, at least one B^{1A} is C₀₋₆alkylene(OH), such as OH. In a seventh embodiment, at least one B^{1A} is oxo (═O).

In one embodiment, BA is not substituted.

In one embodiment, BA is selected from the group consisting of:

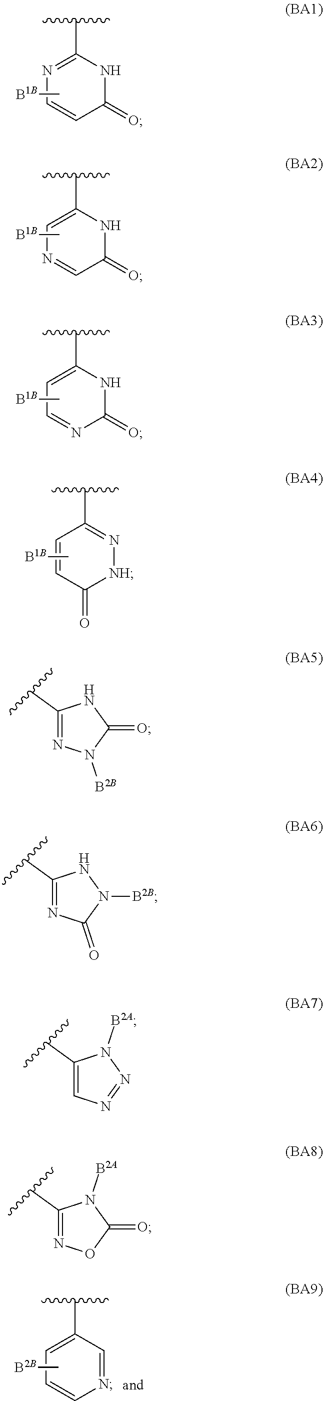

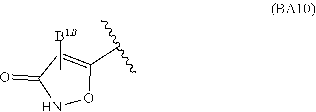

wherein:
B^{1B} is H or C₁₋₆alkyl, such as methyl;
B^{2B} is H or C₁₋₆alkyl, such as methyl; and
B^{2B} is C₀₋₆alkylene(OH), such as OH.

In one embodiment, BA is selected from the group consisting of:

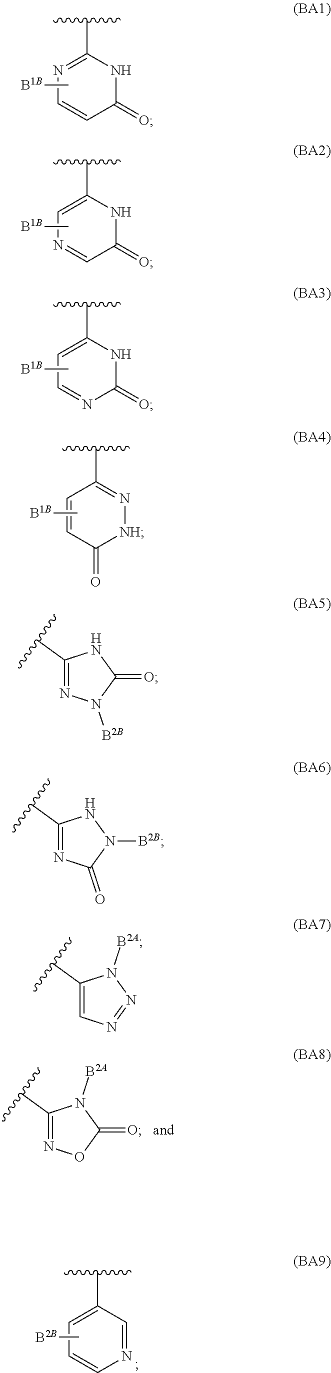

wherein:
B^{1B} is H or C₁₋₆alkyl, such as methyl;
B^{2B} is H or C₁₋₆alkyl, such as methyl; and
B^{2B} is C₀₋₆alkylene(OH), such as OH.

In one embodiment, BA is

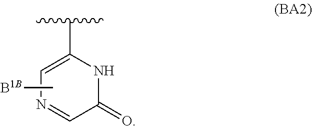

In one embodiment, BA is

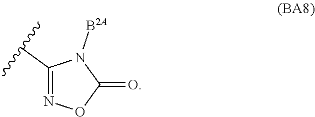

In one embodiment, B^{1B} is H. In a second embodiment, B^{1B} is C₁₋₆alkyl, such as methyl.

In one embodiment, B^{2B} is H. In a second embodiment, B^{2B} is C₁₋₆alkyl, such as methyl.

In one embodiment, the invention provides a compound of formula (IB′):

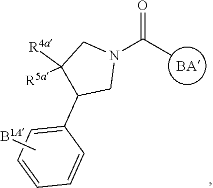

wherein
R^{4a′} is C₁₋₄alkyl;
R^{5a′} is C₁₋₄alkyl; or R^{4a′} and R^{5a′} together with the atom to which they are attached form a C₃₋₆cycloalkyl;
B^{1A′} is halo, such as F; and
BA′ is selected from

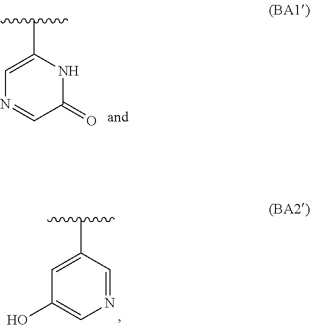 [SMILES: *c1cncc(=O)[nH]1 ; *c1cncc(O)c1]

or a salt and/or solvate thereof.

In one embodiment, R^{4a′} is methyl. In one embodiment, R^{5a′} is methyl. In one embodiment, R^{4a′} and R^{5a′} together with the atom to which they are attached form a cyclobutyl ring.

In one embodiment BA′ is

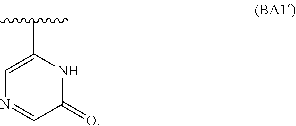 [SMILES: *c1cncc(=O)[nH]1]

In a second embodiment BA′ is

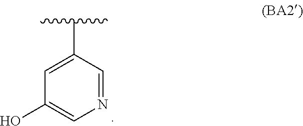 [SMILES: *c1cncc(O)c1]

In the paragraphs that follow, references and preferences set out with respect to the compounds of formula (I) or a salt and/or solvate thereof regarding salts, isomers, processes, pharmaceutical compositions, compounds for use, use and method aspects apply equally to the compounds of formula (IB) or a salt and/or solvate and/or (IB′) or a salt and/or solvate thereof.

In one embodiment, the compound of formula (I) is selected from the group consisting of:
(6-hydroxypyrazin-2-yl)(8-phenyl-6-azaspiro[3.4]octan-6-yl)methanone;
(4-hydroxypyridin-2-yl)(8-phenyl-6-azaspiro[3.4]octan-6-yl)methanone;
(3,3-dimethyl-4-phenylpyrrolidin-1-yl)(5-hydroxypyridin-3-yl)methanone;
2-methyl-5-(8-phenyl-6-azaspiro[3.4]octane-6-carbonyl)-2,4-dihydro-3H-1,2,4-triazol-3-one;
(S)-(6-hydroxypyrazin-2-yl)(8-phenyl-6-azaspiro[3.4]octan-6-yl)methanone;
6-(8-(2-fluorophenyl)-6-azaspiro[3.4]octane-6-carbonyl)pyrazin-2(1H)-one;
6-(3,3-dimethyl-4-phenylpyrrolidine-1-carbonyl)pyrazin-2(1H)-one;
6-(3,3-dimethyl-4-phenylpiperidine-1-carbonyl)pyrazin-2(1H)-one;
(R)-6-(3,3-dimethyl-4-phenylpyrrolidine-1-carbonyl)pyrazin-2(1H)-one;
(S)-6-(3,3-dimethyl-4-phenylpyrrolidine-1-carbonyl)pyrazin-2(1H)-one;
5-(8-(2-fluorophenyl)-6-azaspiro[3.4]octane-6-carbonyl)-2-methyl-2,4-dihydro-3H-1,2,4-triazol-3-one; and
6-(3-methyl-4-phenylpyrrolidine-1-carbonyl)pyrazin-2(1H)-one;
   or a salt and/or solvate of any one thereof.

In another embodiment, the compound of formula (I) is selected from the group consisting of:
(6-hydroxypyrazin-2-yl)(8-phenyl-6-azaspiro[3.4]octan-6-yl)methanone;
(4-hydroxypyridin-2-yl)(8-phenyl-6-azaspiro[3.4]octan-6-yl)methanone;
(3,3-dimethyl-4-phenylpyrrolidin-1-yl)(5-hydroxypyridin-3-yl)methanone;
2-methyl-5-(8-phenyl-6-azaspiro[3.4]octane-6-carbonyl)-2,4-dihydro-3H-1,2,4-triazol-3-one;
(S)-2-methyl-5-(8-phenyl-6-azaspiro[3.4]octane-6-carbonyl)-2,4-dihydro-3H-1,2,4-triazol-3-one;
(R)-2-methyl-5-(8-phenyl-6-azaspiro[3.4]octane-6-carbonyl)-2,4-dihydro-3H-1,2,4-triazol-3-one;
(S)-(6-hydroxypyrazin-2-yl)(8-phenyl-6-azaspiro[3.4]octan-6-yl)methanone;
(R)-(6-hydroxypyrazin-2-yl)(8-phenyl-6-azaspiro[3.4]octan-6-yl)methanone;
6-(8-(2-fluorophenyl)-6-azaspiro[3.4]octane-6-carbonyl)pyrazin-2(1H)-one;
6-(3,3-dimethyl-4-phenylpyrrolidine-1-carbonyl)pyrazin-2(1H)-one;
6-(3,3-dimethyl-4-phenylpiperidine-1-carbonyl)pyrazin-2(1H)-one;
(S)-6-(3,3-dimethyl-4-phenylpyrrolidine-1-carbonyl)pyrazin-2(1H)-one;
(R)-6-(3,3-dimethyl-4-phenylpyrrolidine-1-carbonyl)pyrazin-2(1H)-one;
5-(8-(2-fluorophenyl)-6-azaspiro[3.4]octane-6-carbonyl)-2-methyl-2,4-dihydro-3H-1,2,4-triazol-3-one;
6-(3-methyl-4-phenylpyrrolidine-1-carbonyl)pyrazin-2(1H)-one;
(S)-8-Phenyl-6-(3H-1,2,3-triazole-4-carbonyl)-6-azaspiro[3.4]octane;
(R)-8-Phenyl-6-(3H-1,2,3-triazole-4-carbonyl)-6-azaspiro[3.4]octane;
(R),(R)-6-(3-ethyl-4-phenylpyrrolidine-1-carbonyl)pyrazin-2(1H)-one;
(S),(S)-6-(3-ethyl-4-phenylpyrrolidine-1-carbonyl)pyrazin-2(1H)-one;
(R),(S)-6-(3-ethyl-4-phenylpyrrolidine-1-carbonyl)pyrazin-2(1H)-one;
(S),(R)-6-(3-ethyl-4-phenylpyrrolidine-1-carbonyl)pyrazin-2(1H)-one;
(S)-6-[8-(4-Chlorophenyl)-6-azaspiro[3.4]octane-6-carbonyl]-1H-pyrazin-2-one;
(R)-6-[8-(4-Chlorophenyl)-6-azaspiro[3.4]octane-6-carbonyl]-1H-pyrazin-2-one;
(S)-6-[4-Phenyl-2-azaspiro[4.4]nonane-2-carbonyl]-1H-pyrazin-2-one;
(R)-6-[4-Phenyl-2-azaspiro[4.4]nonane-2-carbonyl]-1H-pyrazin-2-one;
rac-2-{8-phenyl-6-azaspiro[3.4]octane-6-carbonyl}-3H-pyrimidin-4-one;
(S)-6-[(4-Ethynylphenyl)-6-azaspiro[3.4]octane-6-carbonyl]-1H-pyrazin-2-one;
(R)-6-[(4-Ethynylphenyl)-6-azaspiro[3.4]octane-6-carbonyl]-1H-pyrazin-2-one;
(S)-4-[6-(6-oxo-1H-pyrazine-2-carbonyl)-6-azaspiro[3.4]octan-8-yl]benzonitrile;
(R)-4-[6-(6-oxo-1H-pyrazine-2-carbonyl)-6-azaspiro[3.4]octan-8-yl]benzonitrile;
(S)-2-[8-phenyl-6-azaspiro[3.4]octane-6-carbonyl]-3H-pyrimidin-4-one;
(R)-2-[8-phenyl-6-azaspiro[3.4]octane-6-carbonyl]-3H-pyrimidin-4-one;
(3-hydroxyisoxazol-5-yl)(8-phenyl-6-azaspiro[3.4]octan-6-yl)methanone;
(S)-(3-hydroxyisoxazol-5-yl)(8-phenyl-6-azaspiro[3.4]octan-6-yl)methanone;
(R)-(3-hydroxyisoxazol-5-yl)(8-phenyl-6-azaspiro[3.4]octan-6-yl)methanone;
3-[8-Phenyl-6-azaspiro[3.4]octane-6-carbonyl]-4H-1,2,4-oxadiazol-5-one;
(R),(R)-2-(2-fluoro-8-phenyl-6-azaspiro[3.4]octane-6-carbonyl)pyrimidin-4(3H)-one;
(S),(S)-2-(2-fluoro-8-phenyl-6-azaspiro[3.4]octane-6-carbonyl)pyrimidin-4(3H)-one;
(R),(S)-2-(2-fluoro-8-phenyl-6-azaspiro[3.4]octane-6-carbonyl)pyrimidin-4(3H)-one;
(S),(R)-2-(2-fluoro-8-phenyl-6-azaspiro[3.4]octane-6-carbonyl)pyrimidin-4(3H)-one;
(S)-6-(8-phenyl-6-azaspiro[3.4]octane-6-carbonyl)pyridin-2(1H)-one;
(R)-6-(8-phenyl-6-azaspiro[3.4]octane-6-carbonyl)pyridin-2(1H)-one;
(S)-6-(8-phenyl-6-azaspiro[3.4]octane-6-carbonyl)pyrimidin-2(1H)-one;
(R)-6-(8-phenyl-6-azaspiro[3.4]octane-6-carbonyl)pyrimidin-2(1H)-one;
(S)-6-(8-(4-methoxyphenyl)-6-azaspiro[3.4]octane-6-carbonyl)pyrazin-2(1H)-one;
(R)-6-(8-(4-methoxyphenyl)-6-azaspiro[3.4]octane-6-carbonyl)pyrazin-2(1H)-one;
(S)-6-(8-(4-bromophenyl)-6-azaspiro[3.4]octane-6-carbonyl)pyrazin-2(1H)-one;
(R)-6-(8-(4-bromophenyl)-6-azaspiro[3.4]octane-6-carbonyl)pyrazin-2(1H)-one;
(S)-6-(8-(2-chlorophenyl)-6-azaspiro[3.4]octane-6-carbonyl)pyrazin-2(1H)-one;
(R)-6-(8-(2-chlorophenyl)-6-azaspiro[3.4]octane-6-carbonyl)pyrazin-2(1H)-one;
(S)-6-(8-(3-chlorophenyl)-6-azaspiro[3.4]octane-6-carbonyl)pyrazin-2(1H)-one;
(R)-6-(8-(3-chlorophenyl)-6-azaspiro[3.4]octane-6-carbonyl)pyrazin-2(1H)-one;
(S)-6-8-(5-chloro-2-fluorophenyl)-6-azaspiro[3.4]octane-6-carbonyl)pyrazin-2(1H)-one;
(R)-6-8-(5-chloro-2-fluorophenyl)-6-azaspiro[3.4]octane-6-carbonyl)pyrazin-2(1H)-one;
(S)-6-(8-(3-chloro-2-fluorophenyl)-6-azaspiro[3.4]octane-6-carbonyl)pyrazin-2(1H)-one;
(R)-6-(8-(3-chloro-2-fluorophenyl)-6-azaspiro[3.4]octane-6-carbonyl)pyrazin-2(1H)-one;
(S)-3-(8-phenyl-6-azaspiro[3.4]octane-6-carbonyl)-1,2,4-oxadiazol-5(4H)-one;
(R)-3-(8-phenyl-6-azaspiro[3.4]octane-6-carbonyl)-1,2,4-oxadiazol-5(4H)-one;
(S)-6-(8-(4-fluorophenyl)-6-azaspiro[3.4]octane-6-carbonyl)pyrazin-2(1H)-one;
(R)-6-(8-(4-fluorophenyl)-6-azaspiro[3.4]octane-6-carbonyl)pyrazin-2(1H)-one;
(S)-(8-(4-fluorophenyl)-6-azaspiro[3.4]octan-6-yl)(3-hydroxyisoxazol-5-yl)methanone;
(R)-(8-(4-fluorophenyl)-6-azaspiro[3.4]octan-6-yl)(3-hydroxyisoxazol-5-yl)methanone;
(S)-6-(8-(2,4-difluorophenyl)-6-azaspiro[3.4]octane-6-carbonyl)pyrazin-2(1H)-one;
(R)-6-(8-(2,4-difluorophenyl)-6-azaspiro[3.4]octane-6-carbonyl)pyrazin-2(1H)-one;
(S)-(8-(3-fluorophenyl)-6-azaspiro[3.4]octan-6-yl)(3-hydroxyisoxazol-5-yl)methanone;
(R)-(8-(3-fluorophenyl)-6-azaspiro[3.4]octan-6-yl)(3-hydroxyisoxazol-5-yl)methanone;
(S)-(8-(2,3-difluorophenyl)-6-azaspiro[3.4]octan-6-yl)(3-hydroxyisoxazol-5-yl)methanone;
(R)-(8-(2,3-difluorophenyl)-6-azaspiro[3.4]octan-6-yl)(3-hydroxyisoxazol-5-yl)methanone;
(S)-6-(8-(3-fluorophenyl)-6-azaspiro[3.4]octane-6-carbonyl)pyrazin-2(1H)-one;
(R)-6-(8-(3-fluorophenyl)-6-azaspiro[3.4]octane-6-carbonyl)pyrazin-2(1H)-one;
(S)-3-(8-(4-fluorophenyl)-6-azaspiro[3.4]octane-6-carbonyl)-1,2,4-oxadiazol-5(4H)-one;
(R)-3-(8-(4-fluorophenyl)-6-azaspiro[3.4]octane-6-carbonyl)-1,2,4-oxadiazol-5(4H)-one;
(S)-(8-(2,4-difluorophenyl)-6-azaspiro[3.4]octan-6-yl)(3-hydroxyisoxazol-5-yl)methanone;
(R)-(8-(2,4-difluorophenyl)-6-azaspiro[3.4]octan-6-yl)(3-hydroxyisoxazol-5-yl)methanone;
(S)-6-(8-(2,3-difluorophenyl)-6-azaspiro[3.4]octane-6-carbonyl)pyrazin-2(1H)-one;
(R)-6-(8-(2,3-difluorophenyl)-6-azaspiro[3.4]octane-6-carbonyl)pyrazin-2(1H)-one;
(S)-3-(8-(2,3-difluorophenyl)-6-azaspiro[3.4]octane-6-carbonyl)-1,2,4-oxadiazol-5(4H)-one;
(R)-3-(8-(2,3-difluorophenyl)-6-azaspiro[3.4]octane-6-carbonyl)-1,2,4-oxadiazol-5(4H)-one;
(S)-6-(8-(3,4-difluorophenyl)-6-azaspiro[3.4]octane-6-carbonyl)pyrazin-2(1H)-one;
(R)-6-(8-(3,4-difluorophenyl)-6-azaspiro[3.4]octane-6-carbonyl)pyrazin-2(1H)-one;
(S)-(8-(3,4-difluorophenyl)-6-azaspiro[3.4]octan-6-yl)(3-hydroxyisoxazol-5-yl)methanone;
(R)-(8-(3,4-difluorophenyl)-6-azaspiro[3.4]octan-6-yl)(3-hydroxyisoxazol-5-yl)methanone;
(S)-3-(8-(3,4-difluorophenyl)-6-azaspiro[3.4]octane-6-carbonyl)-1,2,4-oxadiazol-5(4H)-one;
(R)-3-(8-(3,4-difluorophenyl)-6-azaspiro[3.4]octane-6-carbonyl)-1,2,4-oxadiazol-5(4H)-one
(S)-3-(8-(2,4-difluorophenyl)-6-azaspiro[3.4]octane-6-carbonyl)-1,2,4-oxadiazol-5(4H)-one;
(R)-3-(8-(2,4-difluorophenyl)-6-azaspiro[3.4]octane-6-carbonyl)-1,2,4-oxadiazol-5(4H)-one;
(S)-3-(8-(3-fluorophenyl)-6-azaspiro[3.4]octane-6-carbonyl)-1,2,4-oxadiazol-5(4H)-one; and
(R)-3-(8-(3-fluorophenyl)-6-azaspiro[3.4]octane-6-carbonyl)-1,2,4-oxadiazol-5(4H)-one.
   or a salt and/or solvate of any one thereof.

The definition of the compounds of formula (I) is intended to include all tautomers of said compounds.

The compounds of the invention may be provided in the form of a pharmaceutically acceptable salt and/or solvate thereof. In particular, the compound of formula (I) may be provided in the form of a pharmaceutically acceptable salt and/or solvate, such as a pharmaceutically acceptable salt. In one embodiment, a compound of formula (I) is provided.

It will be appreciated that for use in medicine the salts of the compounds of formula or (I) should be pharmaceutically acceptable. Non-pharmaceutically acceptable salts of the compounds of formula (I) may be of use in other contexts such as during preparation of the compounds of formula (I). Suitable pharmaceutically acceptable salts will be apparent to those skilled in the art. Pharmaceutically acceptable salts include those described by Berge et al. (1977). Such pharmaceutically acceptable salts include acid and base addition salts. Pharmaceutically acceptable acid additional salts may be formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain compounds of formula (I) may form acid or base addition salts with one or more equivalents of the acid or base. The present invention includes within its scope all possible stoichiometric and non-stoichiometric forms.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

It is to be understood that the present invention encompasses all stereoisomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present disclosure includes all isotopic forms of the compounds of the invention provided herein, whether in a form (I) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³¹Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. 2H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy. In one embodiment, the compounds of the invention are provided in a natural isotopic form. In one embodiment, the compounds of the invention are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of the invention. In one embodiment, the atoms of the compounds of the invention are in an isotopic form which is not radioactive. In one embodiment, one or more atoms of the compounds of the invention are in an isotopic form which is radioactive. Suitably radioactive isotopes are stable isotopes. Suitably the unnatural variant isotopic form is a pharmaceutically acceptable form.

In one embodiment, a compound of the invention is provided whereby a single atom of the compound exists in an unnatural variant isotopic form. In another embodiment, a compound of the invention is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

In general, the compounds of formula (I) may be made according to the organic synthesis techniques known to those skilled in this field, as well as by the representative methods set forth below, those in the Examples, and modifications thereof. In the following schemes, reactive groups can be protected with protecting groups and deprotected according to established techniques well known to the skilled person.

### Generic Routes

Generic routes by which compound examples of the invention may be conveniently prepared are summarised below. In the following description, the groups R^{1a}, R^{1b}, R^{2a}, R^{3a}, R^{4a}, R^{5a}, R^{6a}, BA, AA and x are as defined above for the compounds of formulae (I) and (IB), unless otherwise stated.

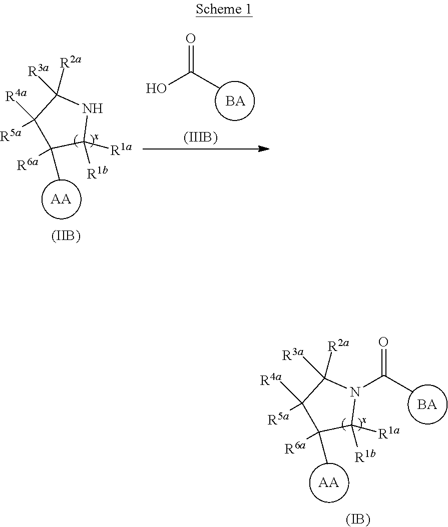

Compounds of formula (I) or (IB) may be prepared by reacting a compound of formula (IIB) with a compound of formula (IIIB) under amide coupling conditions, using an agent such as EDC.HCl (N-ethyl-N′-(3-dimethylaminopropyl)carbodiimide hydrochloride) in a solvent e.g. dimethylformamide at room temperature. Alternative conditions known to the skilled person may be used e.g. HATU (1-[bis(dimethylamino)methylene]-1H-1,2,3-triazolo[4,5-b]pyridinium 3-oxide hexafluorophosphate) or PyBOP (benzotriazol-1-yloxytripyrrolidinophosphonium hexafluorophosphate) in the presence of a base e.g. N,N′-diisopropylethylamine and a solvent e.g. dimethylformamide; or T3P (1-Propanephosphonic anhydride) in the presence of a base e.g. triethylamine and a solvent e.g. dimethylformamide.

Compounds of formulae (IIB) and (IIIB) are commercially available or may be prepared according to methods known to the skilled person.

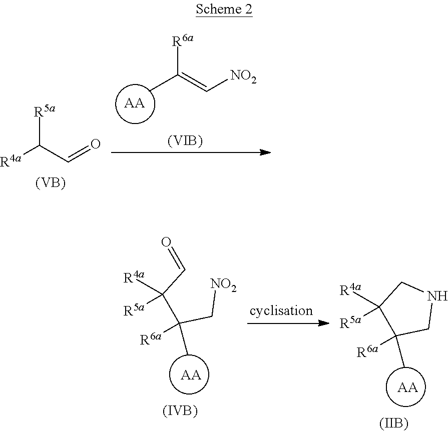

Compounds of formula (IIB) wherein R^{1a}, R^{1b}, R^{2a} and R^{3a} are H and x is 1 may be prepared by reacting a compound of formula (IVB) under metal-mediated cyclisation conditions, using a metal such as zinc in solvent such as a mixture of acetic acid and water. Compounds of formula (IVB) may be obtained by reacting a compound of formula (VB) with a compound of formula (VIB) using an organocatalyst such as L-proline in a solvent such as ethanol.

Compounds of formulae (VB) and (VIB) are commercially available or may be made according to methods known to the skilled person.

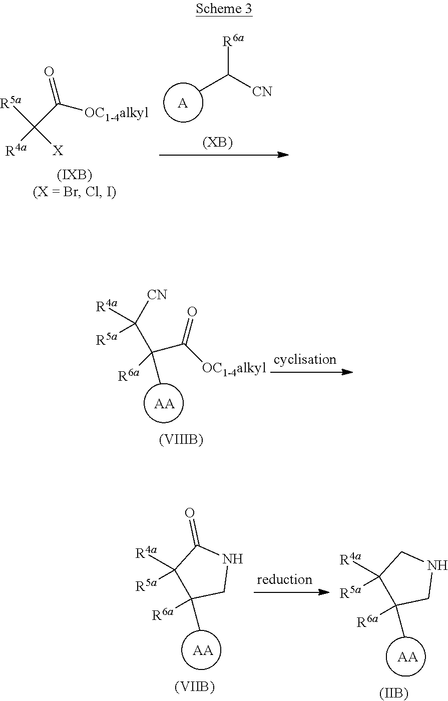

Alternatively, compounds of formula (IIB) wherein R^{1a}, R^{1b}, R^{2a} and R^{3a} are H and x is 1 may be prepared by reacting a compound of formula (VIIB) under reduction conditions e.g. using lithium aluminium hydride in a solvent such as tetrahydrofuran. Compounds of formula (VIIB) may be obtained by reacting a compound of formula (VIIIB) under reduction conditions using e.g. Raney nickel and hydrogen and a solvent such as methanol. Compounds of formula (VIIIB) may be obtained by reacting a compound of formula (IXB) with a compound of formula (XB), using a base such as sodium hydride in a solvent such as dimethylsulfoxide.

Compounds of formulae (IXB) and (XB) are commercially available or may be made according to methods known to the skilled person.

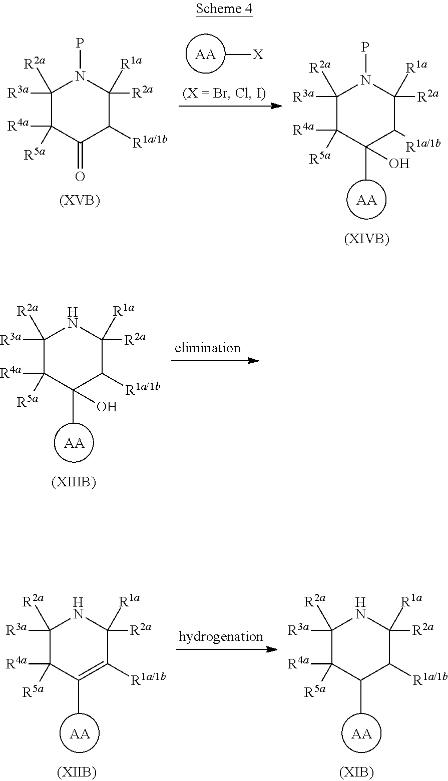

Compounds of formula (IIB) wherein x is 2 may be prepared by reacting a compound of formula (XIB) under hydrogenation conditions e.g. using palladium on carbon and hydrogen. Compounds of formula (XIB) may be prepared via an elimination reaction of compounds of formula (XIIB) using an acid such as p-toluenesulfonic acid in a solvent such as toluene. Compounds of formula (XIIB) may be prepared by reacting a compound of formula (XIVB), wherein P is a nitrogen protecting group e.g. BOC (tert-butyloxycarbonyl) under deprotection conditions e.g. hydrochloric acid in a solvent such as methanol. Compounds of formula (XIVB) may be prepared by reacting a compound of formula (XVB) with a commercially available reagent e.g. bromobenzene, using a base such as n-butyl lithium and a solvent such as tetrahydrofuran.

Compounds of formula (XVB) are commercially available or may be made according to methods known to the skilled person.

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (BOC), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include alkyl esters (such as C₁₋₆ alkyl and C₁₋₆ haloalkyl e.g. C₁₋₄ alkyl esters and C₁₋₄ haloalkyl esters), benzyl esters (including substituted benzyl esters such as p-methoxybenzyl esters), and silyl esters.

### Processes

The invention also provides a process for preparing a compound of formula (I), or salt and/or solvate thereof, which comprises reacting a compound of formula (IIB),

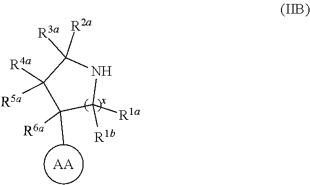

wherein R^{1a}, R^{1b}, R^{2a}, R^{3a}, R^{4a}, R^{5a}, R^{6a}, AA and x are as defined for the compound of formula (I) or a salt thereof,
with a compound of formula (IIIB),

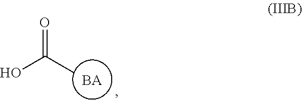

wherein BA is as defined for the compound of formula (I) or a salt thereof.

### Intermediates

The invention also provides novel intermediates used in the preparation of compounds of formula (I). Particular intermediates of interest are those of the following general formulae, wherein the variable groups and associated preferences are as defined previously for compounds of formulae (I). Therefore, in one embodiment the invention provides a compound selected from the group consisting of:
a compound of formula (IIB):

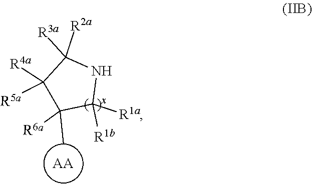

wherein R^{1a}, R^{1b}, R^{2a}, R^{3a}, R^{4a}, R^{5a}, R^{6a} x and AA are as defined for the compound of formula (I);
a compound of formula (IIIB):

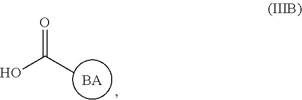

wherein BA is as defined for the compound of formula (I);
a compound of formula (IVB):

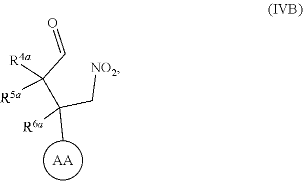

wherein R^{4a}, R^{5a}, R^{6a} and AA are as defined for the compound of formula (I);
a compound of formula (VIIB):

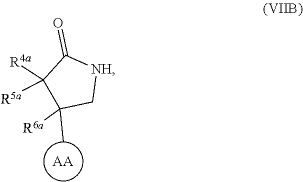

wherein R^{4a}, R^{5a}, R^{6a} and AA are as defined for the compound of formula (I):
a compound of formula (VIIIB):

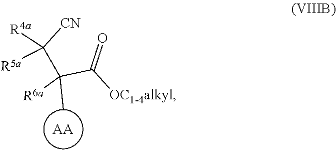

wherein R^{4a}, R^{5a}, R^{6a} and AA are as defined for the compound of formula (I):
a compound of formula (XIB):

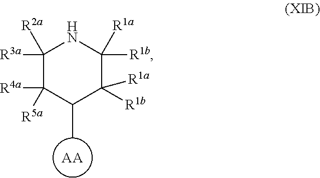

wherein R^{1a}, R^{2a}, R^{1a/b}, R^{2a}, R^{3a}, R^{4a}, R^{5a} and AA are as defined for the compound of formula (I);
a compound of formula (XIIB):

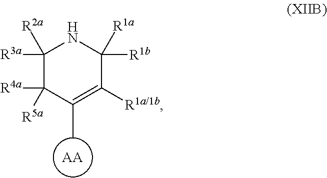

wherein R^{1a}, R^{2a}, R^{1a/b}, R^{2a}, R^{3a}, R^{4a}, R^{5a} and AA are as defined for the compound of formula (I);
a compound of formula (XIIIB):

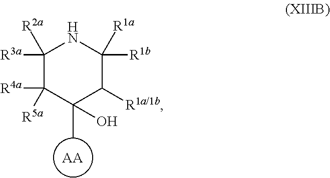

wherein R^{1a}, R^{2a}, R^{1a/b}, R^{2a}, R^{3a}, R^{4a}, R^{5a} and AA are as defined for the compound of formula (I); and
a compound of formula (XIVB):

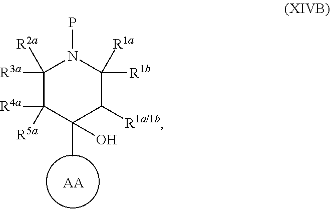

wherein R^{1a}, R^{2a}, R^{1a/b}, R^{2a}, R^{3a}, R^{4a}, R^{5a} and AA are as defined for the compound of formula (I) and P is a nitrogen protecting group such as BOC (tert-butyloxycarbonyl),
or salts, such as pharmaceutically acceptable salts, of any one thereof.

### Therapeutic Methods

The compounds of formula (I) of the present invention have utility as inhibitors of mPTP.

References to compounds of formula (I), or pharmaceutically acceptable salts and/or solvates thereof in the context of therapeutic and prophylactic methods as described below are understood to not include the proviso of formula (I) i.e. (5-hydroxypyridin-3-yl)(8-phenyl-6-azaspiro[3.4]octan-6-yl)methanone:

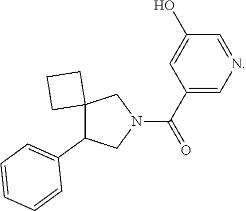 [SMILES: O=C(c1cncc(O)c1)N1CC(c2ccccc2)C2(CCC2)C1]

These compounds are commercially available and to date no utility (e.g. therapeutic utility) of these compounds has been described.

Therefore, the invention provides a compound of formula (I), or a pharmaceutically acceptable salt and/or solvate thereof, for use as a pharmaceutical, in particular in the treatment or prophylaxis of a disease or disorder in which inhibition of mPTP provides a therapeutic or prophylactic effect, for example those diseases and disorders mentioned herein below, wherein the proviso of formula (I) does not apply.

The invention also provides a compound of formula (I), or a pharmaceutically acceptable salt and/or solvate thereof, for use a pharmaceutical, in particular in the treatment of a disease or disorder in which inhibition of mPTP provides a therapeutic effect, for example those diseases and disorders mentioned herein below, wherein the proviso of formula (I) does not apply.

The invention also provides a compound of formula (I), or a pharmaceutically acceptable salt and/or solvate thereof, for use a pharmaceutical, in particular in the prophylaxis of a disease or disorder in which inhibition of mPTP provides a prophylactic effect, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament, in particular for the treatment or prophylaxis of a disease or disorder in which inhibition of mPTP provides a therapeutic or prophylactic effect, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament, in particular for the treatment of a disease or disorder in which inhibition of mPTP provides a therapeutic effect, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament, in particular for the prophylaxis of a disease or disorder in which inhibition of mPTP provides a prophylactic effect, for example those diseases and disorders mentioned herein below, wherein the proviso of formula (I) does not apply.

The invention also provides a method of preventing or treating a disease or disorder in which inhibition of mPTP provides a therapeutic or prophylactic effect in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of treating a disease or disorder in which inhibition of mPTP provides a therapeutic effect in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of preventing a disease or disorder in which inhibition of mPTP provides a prophylactic effect in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The term ‘treatment’ or ‘treating’ as used herein includes the control, mitigation, reduction, or modulation of the disease state or its symptoms.

The term ‘prophylaxis’ or ‘preventing’ is used herein to mean preventing symptoms of a disease or disorder in a subject or preventing recurrence of symptoms of a disease or disorder in an afflicted subject and is not limited to complete prevention of an affliction.

In one embodiment, the disease or disorder is selected from degenerative or neurodegenerative diseases, disorders of the central nervous system, ischemia or re-perfusion injury, metabolic diseases, inflammatory or autoimmune diseases, diseases of aging and renal diseases.

In another embodiment, the disease or disorder is selected from degenerative or neurodegenerative diseases, disorders of the central nervous system, ischemia or re-perfusion injury, metabolic diseases, inflammatory or autoimmune diseases, diseases of aging, renal diseases, hearing loss, a disease or disorder of the eye, Charcot-Marie-Tooth disease (CMT1a) and Leigh syndrome disease.

In one particular embodiment, the disease or disorder is a degenerative or neurodegenerative disease, such as Parkinson's disease, dementia with Lewy bodies, Alzheimer's disease, amyotrophic lateral sclerosis, multiple sclerosis, frontal temporal dementia, chemotherapy induced neuropathy, Huntington's disease, spinocerebellar ataxias, progressive supranuclear palsy, hereditary spastic paraplegia, Duchenne muscular dystrophy, congenital muscular dystrophy, traumatic brain injury (such as concussion) and Friedreich's ataxia. In one preferred embodiment, the disease or disorder is Parkinson's disease. In one preferred embodiment, the disease or disorder is Alzheimer's disease. In one preferred embodiment, the disease or disorder is amyotrophic lateral sclerosis.

In another particular embodiment, the disease or disorder is a disease of the central nervous system, such as AIDS dementia complex, depressive disorders, schizophrenia and epilepsy.

In another embodiment, the disease or disorder is ischemia or re-perfusion injury, such as acute myocardial infarction, stroke, kidney ischemia reperfusion injury, and organ damage during transplantation.

In another embodiment, the disease or disorder is a metabolic disease, such as hepatic steatosis, diabetes, diabetic retinopathy, cognitive decline and other diabetes associated conditions, obesity and feeding behaviours, and non-alcoholic fatty liver disease.

In another embodiment, the disease or disorder is complication linked to a metabolic disease, such as diabetic neuropathy.

In another embodiment, the disease or disorder is an inflammatory or autoimmune disease, such as acute pancreatitis, systemic lupus, organ failure in sepsis and hepatitis.

In another embodiment, the disease or disorder is a disease of aging, such as bone repair, bone weakness in aging in osteoporosis and sarcopenia.

In another embodiment, the disease or disorder is a renal disease, such as chronic kidney disease associated with APOL1 genetic variants and chronic kidney disease.

In another embodiment, the disease or disorder is hearing loss, such as hearing loss due to aging, noise, concussion, traumatic brain injury (TBI), drug induced, and/or genetic hearing loss, including spinal muscular atrophy (SMA) syndrome (SMA1, SMA2, SMA3, and SMA4, also called Type I, II, III and IV).

In another embodiment, the disease or disorder is a disease or disorder of the eye, such as age-related macular degeneration.

In another embodiment, the disease or disorder is Charcot-Marie-Tooth disease (CMT1a).

In another embodiment, the disease or disorder is Leigh syndrome disease.

The compounds of formula (I) are expected to be useful in the treatment or prophylaxis of a mitochondrial disease.

Therefore, the invention provides a compound of formula (I), or a pharmaceutically acceptable salt and/or solvate thereof, for use in the treatment or prophylaxis of a mitochondrial disease, for example those diseases and disorders mentioned herein below, wherein the proviso of formula (I) does not apply.

The invention also provides a compound of formula (I), or a pharmaceutically acceptable salt and/or solvate thereof, for use in the treatment of a mitochondrial disease, for example those diseases and disorders mentioned herein below, wherein the proviso of formula (I) does not apply.

The invention also provides a compound of formula (I), or a pharmaceutically acceptable salt and/or solvate thereof, for use in the prophylaxis of a mitochondrial disease, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the treatment or prophylaxis of a mitochondrial disease, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the treatment of a mitochondrial disease, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the prophylaxis of a mitochondrial disease, for example those diseases and disorders mentioned herein below, wherein the proviso of formula (I) does not apply.

The invention also provides a method of treating or preventing a mitochondrial disease in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of treating a mitochondrial disease in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of preventing a mitochondrial disease in a subject, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

Suitably, the mitochondrial disease is selected from Reye syndrome, Leber's hereditary optic neuropathy and associated disorders and disorders, such as those diseases and disorders disclosed in CA2884607A1 (Stealth Peptides International Inc.) The compounds of formula (I) are expected to be useful in the treatment or prophylaxis of a disease or disorder associated with TDP-43 proteinopathy such as TDP-43 associated neurodegeneration.

Therefore, the invention provides a compound of formula (I), or a pharmaceutically acceptable salt and/or solvate thereof, for use in the treatment or prophylaxis of a disease or disorder associated with TDP-43 proteinopathy such as TDP-43 associated neurodegeneration, for example those diseases and disorders mentioned herein below, wherein the proviso of formula (I) does not apply.

The invention also provides a compound of formula (I), or a pharmaceutically acceptable salt and/or solvate thereof, for use in the treatment of a disease or disorder associated with TDP-43 proteinopathy such as TDP-43 associated neurodegeneration, for example those diseases and disorders mentioned herein below, wherein the proviso of formula (I) does not apply.

The invention also provides a compound of formula (I), or a pharmaceutically acceptable salt and/or solvate thereof, for use in the prophylaxis of a disease or disorder associated with TDP-43 proteinopathy such as TDP-43 associated neurodegeneration, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the treatment or prophylaxis of a disease or disorder associated with TDP-43 proteinopathy such as TDP-43 associated neurodegeneration, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the treatment of a disease or disorder associated with TDP-43 proteinopathy such as TDP-43 associated neurodegeneration, for example those diseases and disorders mentioned herein below, wherein the proviso of formula (I) does not apply.

The invention also provides the use of a compound of formula (I), or a pharmaceutically acceptable salt and/or solvate thereof, in the manufacture of a medicament for the prophylaxis of a disease or disorder associated with TDP-43 proteinopathy such as TDP-43 associated neurodegeneration, for example those diseases and disorders mentioned herein below, wherein the proviso of formula (I) does not apply.

The invention also provides a method of treating or preventing a disease or disorder associated with TDP-43 proteinopathy such as TDP-43 associated neurodegeneration, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of treating a disease or disorder associated with TDP-43 proteinopathy such as TDP-43 associated neurodegeneration, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

The invention also provides a method of preventing a disease or disorder associated with TDP-43 proteinopathy such as TDP-43 associated neurodegeneration, which comprises administering to a subject in need thereof an effective amount of a compound of formula (I), or a pharmaceutically acceptable salt and/or solvate thereof, for example those diseases and disorders mentioned herein below, wherein the proviso of formula (I) does not apply.

Suitably, the disease or disorder associated with TDP-43 proteinopathy such as TDP-43 associated neurodegeneration is selected from Amyotrophic Lateral Sclerosis, Frontotemporal dementia, Facial onset sensory and motor neuronopathy, Primary lateral sclerosis, Progressive muscular atrophy, Inclusion body myopathy associated with early-onset Paget disease of the bone and Frontotemporal lobar degeneration dementia, Perry disease, Chronic traumatic encephalopathy, Severe traumatic brain injury, Alzheimer's disease, Hippocampal sclerosis dementia, Limbic-predominant age-related TDP-43 encephalopathy, and Cerebral age-related TDP-43 with sclerosis.

The invention also provides a compound of formula (I), or a pharmaceutically acceptable salt and/or solvate thereof, for use in the treatment or prophylaxis of a disease or disorder associated with fibrosis, wherein the proviso of formula (I) does not apply.

The invention also provides a compound of formula (I), or a pharmaceutically acceptable salt and/or solvate thereof, for use in the treatment of a disease or disorder associated with fibrosis, wherein the proviso of formula (I) does not apply.

The invention also provides a compound of formula (I), or a pharmaceutically acceptable salt and/or solvate thereof, for use in the prophylaxis of a disease or disorder associated with fibrosis, wherein the proviso of formula (I) does not apply.

The invention also provides the use of a compound of formula (I), or a pharmaceutically acceptable salt and/or solvate thereof, in the manufacture of a medicament for the treatment or prophylaxis of a disease or disorder associated with fibrosis, wherein the proviso of formula (I) does not apply.

The invention also provides the use of a compound of formula (I), or a pharmaceutically acceptable salt and/or solvate thereof, in the manufacture of a medicament for the treatment of a disease or disorder associated with fibrosis, wherein the proviso of formula (I) does not apply.

The invention also provides the use of a compound of formula (I), or a pharmaceutically acceptable salt and/or solvate thereof, in the manufacture of a medicament for the prophylaxis of a disease or disorder associated with fibrosis, wherein the proviso of formula (I) does not apply.

The invention also provides a method of treating or preventing a disease or disorder associated with fibrosis, which comprises administering to a subject in need thereof an effective amount of a compound of formula (I), or a pharmaceutically acceptable salt and/or solvate thereof, wherein the proviso of formula (I) does not apply.

The invention also provides a method of treating a disease or disorder associated with fibrosis, which comprises administering to a subject in need thereof an effective amount of a compound of formula (I), or a pharmaceutically acceptable salt and/or solvate thereof, wherein the proviso of formula (I) does not apply.

The invention also provides a method of preventing a disease or disorder associated with fibrosis, which comprises administering to a subject in need thereof an effective amount of a compound of formula (I), or a pharmaceutically acceptable salt and/or solvate thereof, wherein the proviso of formula (I) does not apply.

Suitably, the disease or disorder associated with fibrosis is selected from chronic kidney disease, idiopathic pulmonary fibrosis, non-alcoholic steatohepatitis, primary biliary cholangitis and systemic sclerosis.

Suitably the subject is a mammal, in particular the subject is a human.

### Pharmaceutical Compositions

For use in therapy the compounds of the invention are usually administered as a pharmaceutical composition. The invention also provides a pharmaceutical composition comprising a compound of formula (I), or a pharmaceutically acceptable salt and/or solvate (e.g. salt) thereof, and a pharmaceutically acceptable carrier or excipient, wherein the proviso of formula (I) does not apply.

In one embodiment, there is provided a pharmaceutical composition comprising a compound of formula (I), or a pharmaceutically acceptable salt and/or solvate (e.g. salt) thereof, for use in the treatment or prophylaxis of a disease or disorder as described herein, wherein the proviso of formula (I) does not apply. In one embodiment, there is provided a pharmaceutical composition comprising a compound of formula (I), or a pharmaceutically acceptable salt and/or solvate (e.g. salt) thereof, for use in the treatment of a disease or disorder as described herein, wherein the proviso of formula (I) does not apply. In one embodiment, there is provided a pharmaceutical composition comprising a compound of formula (I), or a pharmaceutically acceptable salt and/or solvate (e.g. salt) thereof, for use in the prophylaxis of a disease or disorder as described herein, wherein the proviso of formula (I) does not apply.

In a further embodiment, there is provided a method for the treatment or prophylaxis of a disease or disorder as described herein, which comprises administering to a subject in need thereof an effective amount of a pharmaceutical composition comprising a compound of formula (I) or a pharmaceutically acceptable salt and/or solvate (e.g. salt) thereof, wherein the proviso of formula (I) does not apply. In a further embodiment, there is provided a method for the treatment of a disease or disorder as described herein, which comprises administering to a subject in need thereof an effective amount of a pharmaceutical composition comprising a compound of formula (I) or a pharmaceutically acceptable salt and/or solvate (e.g. salt) thereof, wherein the proviso of formula (I) does not apply. In a further embodiment, there is provided a method for the prophylaxis of a disease or disorder as described herein, which comprises administering to a subject in need thereof an effective amount of a pharmaceutical composition comprising a compound of formula (I) or a pharmaceutically acceptable salt and/or solvate (e.g. salt) thereof, wherein the proviso of formula (I) does not apply. Pharmaceutical compositions of the invention may take the form of a pharmaceutical formulation as described below.

The invention also provides the use of a pharmaceutical composition comprising a compound of formula (I), or a pharmaceutically acceptable salt and/or solvate thereof (e.g. salt) thereof, in the manufacture of a medicament for the treatment or prophylaxis of a disease or disorder as described herein, wherein the proviso of formula (I) does not apply. The invention also provides the use of a pharmaceutical composition comprising a compound of formula (I), or a pharmaceutically acceptable salt and/or solvate thereof (e.g. salt) thereof, in the manufacture of a medicament for the treatment of a disease or disorder as described herein, wherein the proviso of formula (I) does not apply. The invention also provides the use of a pharmaceutical composition comprising a compound of formula (I), or a pharmaceutically acceptable salt and/or solvate thereof (e.g. salt) thereof, in the manufacture of a medicament for the prophylaxis of a disease or disorder as described herein, wherein the proviso of formula (I) does not apply.

The amount of active ingredient which is required to achieve a therapeutic effect will, of course, vary with the particular compound, the route of administration, the subject under treatment or prophylaxis, including the type, species, age, weight, sex, and medical condition of the subject and the renal and hepatic function of the subject, and the particular disorder or disease being treated or prevented, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, suitably 0.01 mg per kg of body weight per day (mg/kg/day) to 10 mg/kg/day, and most suitably 0.1 to 5.0 mg/kg/day, for adult humans. For oral administration, the compositions are suitably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, suitably from about 1 mg to about 100 mg of active ingredient. Intravenously, the most suitable doses will range from about 0.1 to about 10 mg/kg/minute during a constant rate infusion. Advantageously, compounds of the invention may be administered in a single daily dose, or the total daily dosage may be administered in divided doses of two, three or four times daily. Furthermore, suitably compounds of the invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The pharmaceutical formulations according to the invention include those suitable for oral, parenteral (including subcutaneous, intradermal, intramuscular, intravenous [bolus or infusion], and intraarticular), intranasal (also known as nasal administration), inhalation (including fine particle dusts or mists which may be generated by means of various types of metered dose pressurized aerosols, nebulizers or insufflators) insufflation, rectal, intraperitoneal, topical (including dermal, buccal, sublingual, and intraocular) and intrathecal administration, although the most suitable route may depend upon, for example, the condition and disorder of the recipient.

Suitable pharmaceutical formulations according to the invention are those suitable for oral, intrathecal and parenteral administration; and more suitably are those suitable for oral or intrathecal administration.

In one suitable embodiment a compound according to formula (I) is administered by intrathecal administration. Such a method of administration involves injection of the compound of the invention into the spinal canal, or into the subarachnoid space so that it reaches the cerebrospinal fluid. This is advantageous for the administration of compounds which may not be able to pass the blood brain barrier via other routes of administration, such as oral administration.

Suitable pharmaceutical formulations may be administered intrathecally by continuous infusion such as with a catheter, or a pump, or intrathecally by a single bolus injection or by intermittent bolus injection. To be administered intrathecally, the pharmaceutical composition may be administered continuously or intermittently. The intermittent administration may be, for example, every thirty minutes, every hour, every several hours, every 24 hours, every couple of days (for example every 48 or 72 hours) or any combination thereof.

When the pharmaceutical formulation of the invention is administered continuously, implantable delivery devices, such as an implantable pump may be employed. Examples of such delivery devices include devices which can be implanted subcutaneously in the body or in the cranium, and provides an access port through which the pharmaceutical formulation may be delivered to the nerves or brain.

Intrathecal dosages of the present invention, when used for the indicated effects, will typically be less than 1 mg, such as less than 500 μg, for example less than 250 μg per kg of body weight when administered in a single dose or intermittently for adult humans. When administered continuously, the intrathecal dosages of the present invention will typically be less than 250 μg per kg body weight per hour, such as less than 125 μg per kg body weight per hour for adult humans.

In another suitable embodiment a compound according to formula (I) is administered by intranasal, inhalation (including fine particle dusts or mists which may be generated by means of various types of metered dose pressurized aerosols, nebulizers or insufflators) or insufflation administration. Such a method of administration allows for low doses of the compound of the invention to be administered, which can lead to a reduction in side-effects. For example, a daily dose of 10 to 0.01 μg, suitably 1 to 0.01 μg, and more suitably in the region of as low as 0.1 μg (100 ng) of compound of the invention may be used.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The compounds of formula (I) can, for example, be administered in a form suitable for immediate release or extended release. Immediate release or extended release can be achieved by the use of suitable pharmaceutical compositions comprising a compound of the present invention, or, particularly in the case of extended release, by the use of devices such as subcutaneous implants or osmotic pumps. The compounds of the invention may also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of formula (I) can also be delivered through the oral cavity by sublingual and/or buccal administration. Molded tablets, compressed tablets or freeze-dried tablets are exemplary forms which may be used. Exemplary compositions include those formulating a compound of the present invention with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds of formula (I) can also be administered in the form of liposome delivery systems, such as small unilamellar vesicles, large unilamellar vesicles and multilamellar vesicles. Liposomes can be formed from a variety of phospholipids, 1,2-dipalmitoylphosphatidylcholine, phosphatidyl ethanolamine (cephaline), or phosphatidylcholine (lecithin).

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for intranasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Suitable unit dosage formulations are those containing an effective dose, as hereinbefore recited, or an appropriate fraction thereof, of the active ingredient. It should be understood that in addition to the ingredients particularly mentioned above, the formulations of this invention may include other agents conventional in the art having regard to the type of formulation in question, for example those suitable for oral administration may include flavouring agents.

The compounds of formula (I) are expected to display the advantageous property of inhibitory activity of mPTP as demonstrated in the assays of Biological Example 1 (preferably with a pIC₅₀ value of 6.0 and above in the rat liver assay and/or with a pIC₅₀ value of 7.0 and above in the rat brain assay).

### CLAUSES

The invention is further defined by the following clauses.

Clause 1. A compound according to formula (I):

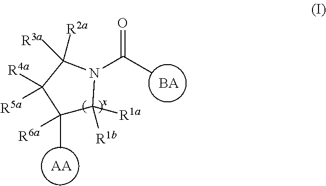

wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is H, C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the carbon atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl optionally substituted by one or more AA¹;
   AA¹ is halo, C₂₋₆alkynyl, C₁₋₆alkyl, C₁₋₆haloalkyl, C₁₋₆alkoxy, C₁₋₆haloalkoxy, C₃₋₆ cycloalkyl, CN, OH, NR^{q}R^{r}, NHSO₂R^{t};
      R^{q} is H or C₁₋₄alkyl;
      R^{r} is H or C₁₋₄alkyl;
      R^{t} is C₁₋₄alkyl;
   BA is a monocyclic or bicyclic heterocycle or a monocyclic or bicyclic heteroaryl wherein said heterocycle or heteroaryl may be optionally substituted by one or more B^{1A}; and
      B^{1A} is halo, C₁₋₆alkyl, C₁₋₆haloalkyl, oxo (C═O), C₁₋₆alkoxy, C₁₋₆haloalkoxy, or C₀₋₆ alkylene(OH);
         or a salt and/or solvate thereof,
         and provided that the compound of formula (I) is not 5-hydroxypyridin-3-yl)(8-phenyl-6-azaspiro[3.4]octan-6-yl)methanone:

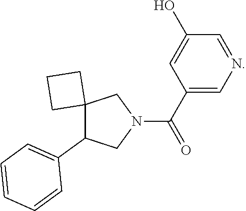 [SMILES: O=C(c1cncc(O)c1)N1CC(c2ccccc2)C2(CCC2)C1]

Clause 2. The compound of formula (IB) according to claim 1:

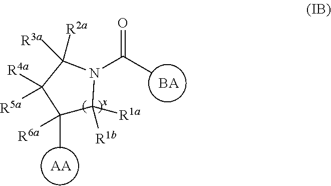

wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is H, C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl optionally substituted by one or more AA¹;
   AA¹ is halo, C₁₋₆alkyl, C₁₋₆haloalkyl, C₁₋₆alkoxy, C₁₋₆haloalkoxy, C₃₋₆cycloalkyl, CN, OH, NR^{q}R^{r}, NHSO₂R^{t};
      R^{q} is H or C₁₋₄alkyl;
      R^{r} is H or C₁₋₄alkyl;
      R^{t} is C₁₋₄alkyl;
   BA is a monocyclic or bicyclic heterocycle or a monocyclic or bicyclic heteroaryl which may be optionally substituted by one or more B^{1A}; and
      B^{1A} is halo, C₁₋₆alkyl, C₁₋₆haloalkyl, C₁₋₆alkoxy, C₁₋₆haloalkoxy, or C₀₋₆alkylene(OH);
         or a salt and/or solvate thereof,
         and provided that the compound of formula (IB) is not 5-hydroxypyridin-3-yl)(8-phenyl-6-azaspiro[3.4]octan-6-yl)methanone:

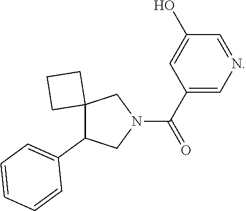 [SMILES: O=C(c1cncc(O)c1)N1CC(c2ccccc2)C2(CCC2)C1]

Clause 3. The compound or salt and/or solvate according to clause 1 or clause 2 which is the compound or pharmaceutically acceptable salt and/or solvate thereof.

Clause 4. The pharmaceutically acceptable salt and solvate according to clause 3.

Clause 5. The pharmaceutically acceptable salt according to clause 3.

Clause 6. The pharmaceutically acceptable solvate according to clause 3.

Clause 7. The compound according to clause 1.

Clause 8. The compound or salt and/or solvate thereof according to any one of clauses 1 to 7, which is a compound according to formula (IB):

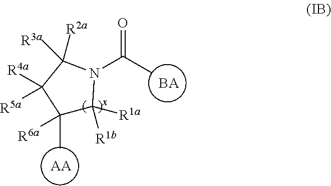

or a salt and/or solvate thereof.

Clause 9. The compound or salt and/or solvate thereof according to any one of clauses 1, 2 or 8, wherein R^{1a} is H.

Clause 10. The compound or salt and/or solvate thereof according to any one of clauses 1, 2 or 8, wherein R^{1a} is C₁₋₄alkyl, such as methyl.

Clause 11. The compound or salt and/or solvate thereof according to any one of clauses 1, 2 or 8 to 10, wherein R^{1b} is H.

Clause 12. The compound or salt and/or solvate thereof according to any one of clauses 1, 2 or 8 to 10, wherein R^{1b} is C₁₋₄alkyl, such as methyl.

Clause 13. The compound or salt and/or solvate thereof according to any one of clauses 1, 2 or 8 to 12, wherein R^{2a} is H.

Clause 14. The compound or salt and/or solvate thereof according to any one of clauses 1, 2 or 8 to 12, wherein R^{2a} is halo.

Clause 15. The compound or salt and/or solvate thereof according to any one of clauses 1, 2 or 8 to 12, wherein R^{2a} is C₁₋₄alkyl.

Clause 16. The compound or salt and/or solvate thereof according to any one of clauses 1, 2 or 8 to 12, wherein R^{2a} is C₁₋₄haloalkyl.

Clause 17. The compound or salt and/or solvate thereof according to any one of clauses 1, 2 or 8 to 16, wherein R^{3a} is H.

Clause 18. The compound or salt and/or solvate thereof according to any one of clauses 1, 2 or 8 to 16, wherein R^{3a} is halo.

Clause 19. The compound or salt and/or solvate thereof according to any one of clauses 1, 2 or 8 to 16, wherein R^{3a} is C₁₋₄alkyl.

Clause 20. The compound or salt and/or solvate thereof according to any one of clauses 1, 2 or 8 to 19, wherein R^{4a} is H.

Clause 21. The compound or salt and/or solvate thereof according to any one of clauses 1, 2 or 8 to 19, wherein R^{4a} is C₁₋₄alkyl, such as methyl.

Clause 22. The compound or salt and/or solvate thereof according to any one of clauses 1, 2 or 8 to 21, wherein R^{5a} is H.

Clause 23. The compound or salt and/or solvate thereof according to any one of clauses 1, 2 or 8 to 21, wherein R^{5a} is C₁₋₆alkyl, such as methyl.

Clause 24. The compound or salt and/or solvate thereof according to any one of clauses 1, 2 or 8 to 21, wherein R^{5a} is C₁₋₆haloalkyl.

Clause 25. The compound or salt and/or solvate thereof according to any one of clauses 1, 2 or 8 to 21, wherein R^{5a} is C₁₋₆alkoxy.

Clause 26. The compound or salt and/or solvate thereof according to any one of clauses 1, 2 or 8 to 21, wherein R^{5a} is C₁₋₆haloalkoxy.

Clause 27. The compound or salt and/or solvate thereof according to any one of clauses 1, 2 or 8 to 21, wherein R^{5a} is C₂₋₆alkenyl.

Clause 28. The compound or salt and/or solvate thereof according to any one of clauses 1, 2 or 8 to 21, wherein R^{5a} is C₂₋₆haloalkenyl.

Clause 29. The compound or salt and/or solvate thereof according to any one of clauses 1, 2 or 8 to 21, wherein R^{5a} is C₀₋₆alkylene(C₃₋₆cycloalkyl).

Clause 30. The compound or salt and/or solvate thereof according to any one of clauses 1, 2 or 8 to 21, wherein R^{5a} is C₀₋₆alkylene(OH).

Clause 31. The compound or salt and/or solvate thereof according to any one of clauses 1, 2 or 8 to 19, wherein R^{4a} and R^{5a} together with the atom to which they are attached form a C₃₋₆ cycloalkyl, for example cyclobutyl.

Clause 32. The compound or salt and/or solvate thereof according to any one of clauses 1, 2 or 8 to 31, wherein R^{6a} is H.

Clause 33. The compound or salt and/or solvate thereof according to any one of clauses 1, 2 or 8 to 31, wherein R^{6a} is halo.

Clause 34. The compound or salt and/or solvate thereof according to any one of clauses 1, 2 or 8 to 31, wherein R^{6a} is C₁₋₄alkyl.

Clause 35. The compound or salt and/or solvate thereof according to any one of clauses 1, 2 or 8 to 31, wherein R^{6a} is C₁₋₄haloalkyl.

Clause 36. The compound or salt and/or solvate thereof according to any one of clauses 1, 2 or 8 to 31, wherein R^{6a} is C₁₋₄alkoxy.

Clause 37. The compound or salt and/or solvate thereof according to any one of clauses 1, 2 or 8 to 31, wherein R^{6a} is C₁₋₄haloalkoxy.

Clause 38. The compound or salt and/or solvate thereof according to any one of clauses 1, 2 or 8 to 37, wherein x is 1.

Clause 39. The compound or salt and/or solvate thereof according to any one of clauses 1, 2 or 8 to 37, wherein x is 2.

Clause 40. The compound or salt and/or solvate thereof according to any one of clauses 1, 2 or 8 to 39, wherein AA is phenyl.

Clause 41. The compound or salt and/or solvate thereof according to any one of clauses 1, 2 or 8 to 39, wherein AA is phenyl fused to C₅₋₆cycloalkyl.

Clause 42. The compound or salt and/or solvate thereof according to clause 40 or 41, wherein AA is substituted by one or more (such as one two or three, for example one or two, particularly one) AA¹.

Clause 43. The compound or salt and/or solvate thereof according to clause 42, wherein at least one AA¹ is halo.

Clause 44. The compound or salt and/or solvate thereof according to clause 43, wherein at least one AA¹ is F.

Clause 45. The compound or salt and/or solvate thereof according to any one of clauses 42 to 44, wherein at least one AA¹ is C₂₋₆alkynyl.

Clause 46. The compound or salt and/or solvate thereof according to any one of clauses 42 to 45, wherein at least one AA¹ is C₁₋₆alkyl.

Clause 47. The compound or salt and/or solvate thereof according to any one of clauses 42 to 46, wherein at least one AA¹ is C₁₋₆haloalkyl.

Clause 48. The compound or salt and/or solvate thereof according to any one of clauses 42 to 47, wherein at least one AA¹ is C₁₋₆alkoxy.

Clause 49. The compound or salt and/or solvate thereof according to any one of clauses 42 to 48, wherein at least one AA¹ is C₁₋₆haloalkoxy.

Clause 50. The compound or salt and/or solvate thereof according to any one of clauses 42 to 49, wherein at least one AA¹ is C₃₋₆cycloalkyl.

Clause 51. The compound or salt and/or solvate thereof according to any one of clauses 42 to 50, wherein at least one AA¹ is CN.

Clause 52. The compound or salt and/or solvate thereof according to any one of clauses 42 to 51, wherein at least one AA¹ is NR^{q}R^{r}.

Clause 53. The compound or salt and/or solvate thereof according to clause 52, wherein R^{q} is H.

Clause 54. The compound or salt and/or solvate thereof according to clause 52, wherein R^{q} is C₁₋₄alkyl.

Clause 55. The compound or salt and/or solvate thereof according to any one of clauses 42 to 54, wherein at least one AA¹ is NHSO₂R^{t}.

Clause 56. The compound or salt and/or solvate thereof according to clause 40 or 41, wherein AA is not substituted.

Clause 57. The compound or salt and/or solvate thereof according to any one of clauses 1, 2 or 8 to 56, wherein BA is a monocyclic heterocycle.

Clause 58. The compound or salt and/or solvate thereof according to clause 57, wherein BA is a 5-membered monocyclic heterocycle.

Clause 59. The compound or salt and/or solvate thereof according to any one of clauses 1, 2 or 8 to 56, wherein BA is a bicyclic heterocycle.

Clause 60. The compound or salt and/or solvate thereof according to any one of clauses 1, 2 or 8 to 56, wherein BA is a monocyclic heteroaryl.

Clause 61. The compound or salt and/or solvate thereof according to clause 60, wherein BA is a 6-membered monocyclic heteroaryl.

Clause 62. The compound or salt and/or solvate thereof according to any one of clauses 1, 2 or 8 to 56, wherein BA is a bicyclic heteroaryl.

Clause 63. The compound or salt and/or solvate thereof according to any one of clauses 57 to 62, wherein BA is substituted by one or more (such as one, two or three, for example one or two, particularly one) B^{1A}.

Clause 64. The compound or salt and/or solvate thereof according to clause 63, wherein at least one B^{1A} is halo.

Clause 65. The compound or salt and/or solvate thereof according to clause 63 or clause 64, wherein at least one B^{1A} is C₁₋₆alkyl.

Clause 66. The compound or salt and/or solvate thereof according to clause 63 or clause 65, wherein at least one B^{1A} is methyl.

Clause 67. The compound or salt and/or solvate thereof according to any one of clauses 63 to 66, wherein at least one B^{1A} is C₁₋₆haloalkyl.

Clause 68. The compound or salt and/or solvate thereof according to any one of clauses 63 to 67, wherein at least one B^{1A} is oxo (C═O).

Clause 69. The compound or salt and/or solvate thereof according to any one of clauses 63 to 68, wherein at least one B^{1A} is C₁₋₆alkoxy.

Clause 70. The compound or salt and/or solvate thereof according to any one of clauses 63 to 69, wherein at least one B^{1A} is C₁₋₆haloalkoxy.

Clause 71. The compound or salt and/or solvate thereof according to any one of clauses 63 to 70, wherein at least one B^{1A} is C₀₋₆alkylene(OH), such as OH.

Clause 72. The compound or salt and/or solvate thereof according to any one of clauses 57 to 62, wherein BA is not substituted.

Clause 73. The compound or salt and/or solvate thereof according to any one of clauses 1, 2 or 8 to 72, wherein BA is selected from the group consisting of:

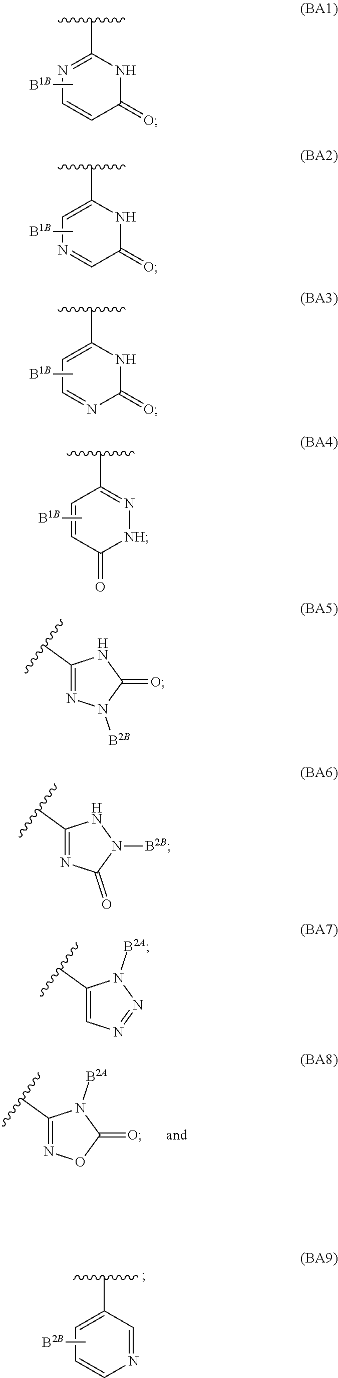

wherein:
B^{1B} is H or C₁₋₆alkyl, such as methyl;
B^{2B} is H or C₁₋₆alkyl, such as methyl; and
B^{2B} is C₀₋₆alkylene(OH), such as OH.

Clause 74. The compound or salt and/or solvate thereof according to clause 73, wherein BA is:

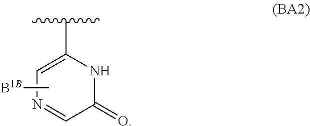

Clause 75. The compound or salt and/or solvate thereof according to clause 73, wherein BA is:

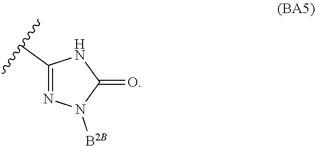

Clause 76. The compound or salt and/or solvate thereof according to clause 73, wherein BA is:

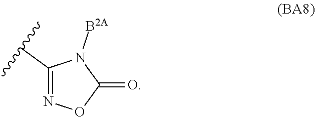

Clause 77. The compound or salt and/or solvate thereof according to any one of clauses 1, 2 or 8, which is a compound of formula (IB′):

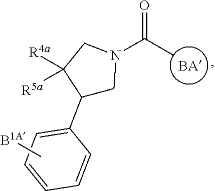

wherein
R^{4a′} is C₁₋₄alkyl;
R^{5a′} is C₁₋₄alkyl; or R^{4a′} and R^{5a′} together with the atom to which they are attached form a C₃₋₆ cycloalkyl;
B^{1A′} is halo, such as F; and
BA′ is selected from

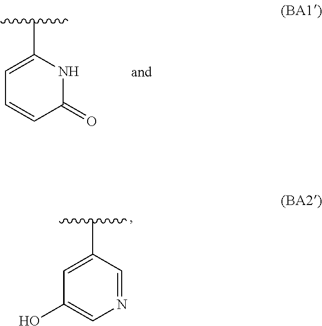 [SMILES: *c1cccc(=O)[nH]1 ; *c1cncc(O)c1]

or a salt and/or solvate thereof.

Clause 78. The compound or salt and/or solvate thereof according to clause 77, wherein R^{4a′} and R^{5a′} together with the atom to which they are attached form a cyclobutyl ring.

Clause 79. The compound or salt and/or solvate thereof according to clause 77 or clause 78,

wherein BA′ is

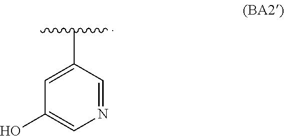 [SMILES: *c1cncc(O)c1]

Clause 80. The compound or salt and/or solvate thereof according to any one of clauses 1, 2 or 8, selected from the group consisting of:
(6-hydroxypyrazin-2-yl)(8-phenyl-6-azaspiro[3.4]octan-6-yl)methanone;
(4-hydroxypyridin-2-yl)(8-phenyl-6-azaspiro[3.4]octan-6-yl)methanone;
(3,3-dimethyl-4-phenylpyrrolidin-1-yl)(5-hydroxypyridin-3-yl)methanone;
2-methyl-5-(8-phenyl-6-azaspiro[3.4]octane-6-carbonyl)-2,4-dihydro-3H-1,2,4-triazol-3-one;
(S)-(6-hydroxypyrazin-2-yl)(8-phenyl-6-azaspiro[3.4]octan-6-yl)methanone;
6-(8-(2-fluorophenyl)-6-azaspiro[3.4]octane-6-carbonyl)pyrazin-2(1H)-one;
6-(3,3-dimethyl-4-phenylpyrrolidine-1-carbonyl)pyrazin-2(1H)-one;
6-(3,3-dimethyl-4-phenylpiperidine-1-carbonyl)pyrazin-2(1H)-one;
(R)-6-(3,3-dimethyl-4-phenylpyrrolidine-1-carbonyl)pyrazin-2(1H)-one;
(S)-6-(3,3-dimethyl-4-phenylpyrrolidine-1-carbonyl)pyrazin-2(1H)-one;
5-(8-(2-fluorophenyl)-6-azaspiro[3.4]octane-6-carbonyl)-2-methyl-2,4-dihydro-3H-1,2,4-triazol-3-one; and
6-(3-methyl-4-phenylpyrrolidine-1-carbonyl)pyrazin-2(1H)-one;
   or a salt and/or solvate of any one thereof.

Clause 81. The compound, salt and/or solvate thereof according to any one of clauses 1, 2 or 8, selected from the group consisting of:
(6-hydroxypyrazin-2-yl)(8-phenyl-6-azaspiro[3.4]octan-6-yl)methanone;
(4-hydroxypyridin-2-yl)(8-phenyl-6-azaspiro[3.4]octan-6-yl)methanone;
(3,3-dimethyl-4-phenylpyrrolidin-1-yl)(5-hydroxypyridin-3-yl)methanone;
2-methyl-5-(8-phenyl-6-azaspiro[3.4]octane-6-carbonyl)-2,4-dihydro-3H-1,2,4-triazol-3-one;
(S)-2-methyl-5-(8-phenyl-6-azaspiro[3.4]octane-6-carbonyl)-2,4-dihydro-3H-1,2,4-triazol-3-one;
(R)-2-methyl-5-(8-phenyl-6-azaspiro[3.4]octane-6-carbonyl)-2,4-dihydro-3H-1,2,4-triazol-3-one;
(S)-(6-hydroxypyrazin-2-yl)(8-phenyl-6-azaspiro[3.4]octan-6-yl)methanone;
(R)-(6-hydroxypyrazin-2-yl)(8-phenyl-6-azaspiro[3.4]octan-6-yl)methanone;
6-(8-(2-fluorophenyl)-6-azaspiro[3.4]octane-6-carbonyl)pyrazin-2(1H)-one;
6-(3,3-dimethyl-4-phenylpyrrolidine-1-carbonyl)pyrazin-2(1H)-one;
6-(3,3-dimethyl-4-phenylpiperidine-1-carbonyl)pyrazin-2(1H)-one;
(S)-6-(3,3-dimethyl-4-phenylpyrrolidine-1-carbonyl)pyrazin-2(1H)-one;
(R)-6-(3,3-dimethyl-4-phenylpyrrolidine-1-carbonyl)pyrazin-2(1H)-one;
5-(8-(2-fluorophenyl)-6-azaspiro[3.4]octane-6-carbonyl)-2-methyl-2,4-dihydro-3H-1,2,4-triazol-3-one;
6-(3-methyl-4-phenylpyrrolidine-1-carbonyl)pyrazin-2(1H)-one;
(S)-8-Phenyl-6-(3H-1,2,3-triazole-4-carbonyl)-6-azaspiro[3.4]octane;
(R)-8-Phenyl-6-(3H-1,2,3-triazole-4-carbonyl)-6-azaspiro[3.4]octane;
(R), (R)-6-(3-ethyl-4-phenylpyrrolidine-1-carbonyl)pyrazin-2(1H)-one;
(S),(S)-6-(3-ethyl-4-phenylpyrrolidine-1-carbonyl)pyrazin-2(1H)-one;
(R), (S)-6-(3-ethyl-4-phenylpyrrolidine-1-carbonyl) pyrazin-2(1H)-one;
(S),(R)-6-(3-ethyl-4-phenylpyrrolidine-1-carbonyl)pyrazin-2(1H)-one;
(S)-6-[8-(4-Chlorophenyl)-6-azaspiro[3.4]octane-6-carbonyl]-1H-pyrazin-2-one;
(R)-6-[8-(4-Chlorophenyl)-6-azaspiro[3.4]octane-6-carbonyl]-1H-pyrazin-2-one;
(S)-6-[4-Phenyl-2-azaspiro[4.4]nonane-2-carbonyl]-1H-pyrazin-2-one;
(R)-6-[4-Phenyl-2-azaspiro[4.4]nonane-2-carbonyl]-1H-pyrazin-2-one;
rac-2-{8-phenyl-6-azaspiro[3.4]octane-6-carbonyl}-3H-pyrimidin-4-one;
(S)-6-[(4-Ethynylphenyl)-6-azaspiro[3.4]octane-6-carbonyl]-1H-pyrazin-2-one;
(R)-6-[(4-Ethynylphenyl)-6-azaspiro[3.4]octane-6-carbonyl]-1H-pyrazin-2-one;
(S)-4-[6-(6-oxo-1H-pyrazine-2-carbonyl)-6-azaspiro[3.4]octan-8-yl]benzonitrile;
(R)-4-[6-(6-oxo-1H-pyrazine-2-carbonyl)-6-azaspiro[3.4]octan-8-yl]benzonitrile;
(S)-2-[8-phenyl-6-azaspiro[3.4]octane-6-carbonyl]-3H-pyrimidin-4-one;
(R)-2-[8-phenyl-6-azaspiro[3.4]octane-6-carbonyl]-3H-pyrimidin-4-one;
(3-hydroxyisoxazol-5-yl)(8-phenyl-6-azaspiro[3.4]octan-6-yl)methanone;
(S)-(3-hydroxyisoxazol-5-yl)(8-phenyl-6-azaspiro[3.4]octan-6-yl)methanone;
(R)-(3-hydroxyisoxazol-5-yl)(8-phenyl-6-azaspiro[3.4]octan-6-yl)methanone;
3-[8-Phenyl-6-azaspiro[3.4]octane-6-carbonyl]-4H-1,2,4-oxadiazol-5-one;
(R),(R)-2-(2-fluoro-8-phenyl-6-azaspiro[3.4]octane-6-carbonyl)pyrimidin-4(3H)-one;
(S),(S)-2-(2-fluoro-8-phenyl-6-azaspiro[3.4]octane-6-carbonyl)pyrimidin-4(3H)-one;
(R),(S)-2-(2-fluoro-8-phenyl-6-azaspiro[3.4]octane-6-carbonyl)pyrimidin-4(3H)-one;
(S),(R)-2-(2-fluoro-8-phenyl-6-azaspiro[3.4]octane-6-carbonyl)pyrimidin-4(3H)-one;
(S)-6-(8-phenyl-6-azaspiro[3.4]octane-6-carbonyl)pyridin-2(1H)-one;
(R)-6-(8-phenyl-6-azaspiro[3.4]octane-6-carbonyl)pyridin-2(1H)-one;
(S)-6-(8-phenyl-6-azaspiro[3.4]octane-6-carbonyl)pyrimidin-2(1H)-one;
(R)-6-(8-phenyl-6-azaspiro[3.4]octane-6-carbonyl)pyrimidin-2(1H)-one;
(S)-6-(8-(4-methoxyphenyl)-6-azaspiro[3.4]octane-6-carbonyl)pyrazin-2(1H)-one;
(R)-6-(8-(4-methoxyphenyl)-6-azaspiro[3.4]octane-6-carbonyl)pyrazin-2(1H)-one;
(S)-6-(8-(4-bromophenyl)-6-azaspiro[3.4]octane-6-carbonyl)pyrazin-2(1H)-one;
(R)-6-(8-(4-bromophenyl)-6-azaspiro[3.4]octane-6-carbonyl)pyrazin-2(1H)-one;
(S)-6-(8-(2-chlorophenyl)-6-azaspiro[3.4]octane-6-carbonyl)pyrazin-2(1H)-one;
(R)-6-(8-(2-chlorophenyl)-6-azaspiro[3.4]octane-6-carbonyl)pyrazin-2(1H)-one;
(S)-6-(8-(3-chlorophenyl)-6-azaspiro[3.4]octane-6-carbonyl)pyrazin-2(1H)-one;
(R)-6-(8-(3-chlorophenyl)-6-azaspiro[3.4]octane-6-carbonyl)pyrazin-2(1H)-one;
(S)-6-8-(5-chloro-2-fluorophenyl)-6-azaspiro[3.4]octane-6-carbonyl)pyrazin-2(1H)-one;
(R)-6-8-(5-chloro-2-fluorophenyl)-6-azaspiro[3.4]octane-6-carbonyl)pyrazin-2(1H)-one;
(S)-6-(8-(3-chloro-2-fluorophenyl)-6-azaspiro[3.4]octane-6-carbonyl)pyrazin-2(1H)-one;
(R)-6-(8-(3-chloro-2-fluorophenyl)-6-azaspiro[3.4]octane-6-carbonyl)pyrazin-2(1H)-one;
(S)-3-(8-phenyl-6-azaspiro[3.4]octane-6-carbonyl)-1,2,4-oxadiazol-5(4H)-one;
(R)-3-(8-phenyl-6-azaspiro[3.4]octane-6-carbonyl)-1,2,4-oxadiazol-5(4H)-one;
(S)-6-(8-(4-fluorophenyl)-6-azaspiro[3.4]octane-6-carbonyl)pyrazin-2(1H)-one;
(R)-6-(8-(4-fluorophenyl)-6-azaspiro[3.4]octane-6-carbonyl)pyrazin-2(1H)-one;
(S)-(8-(4-fluorophenyl)-6-azaspiro[3.4]octan-6-yl)(3-hydroxyisoxazol-5-yl)methanone;
(R)-(8-(4-fluorophenyl)-6-azaspiro[3.4]octan-6-yl)(3-hydroxyisoxazol-5-yl)methanone;
(S)-6-(8-(2,4-difluorophenyl)-6-azaspiro[3.4]octane-6-carbonyl)pyrazin-2(1H)-one;
(R)-6-(8-(2,4-difluorophenyl)-6-azaspiro[3.4]octane-6-carbonyl)pyrazin-2(1H)-one;
(S)-(8-(3-fluorophenyl)-6-azaspiro[3.4]octan-6-yl)(3-hydroxyisoxazol-5-yl)methanone;
(R)-(8-(3-fluorophenyl)-6-azaspiro[3.4]octan-6-yl)(3-hydroxyisoxazol-5-yl)methanone;
(S)-(8-(2,3-difluorophenyl)-6-azaspiro[3.4]octan-6-yl)(3-hydroxyisoxazol-5-yl)methanone;
(R)-(8-(2,3-difluorophenyl)-6-azaspiro[3.4]octan-6-yl)(3-hydroxyisoxazol-5-yl)methanone;
(S)-6-(8-(3-fluorophenyl)-6-azaspiro[3.4]octane-6-carbonyl)pyrazin-2(1H)-one;
(R)-6-(8-(3-fluorophenyl)-6-azaspiro[3.4]octane-6-carbonyl)pyrazin-2(1H)-one;
(S)-3-(8-(4-fluorophenyl)-6-azaspiro[3.4]octane-6-carbonyl)-1,2,4-oxadiazol-5(4H)-one;
(R)-3-(8-(4-fluorophenyl)-6-azaspiro[3.4]octane-6-carbonyl)-1,2,4-oxadiazol-5(4H)-one;
(S)-(8-(2,4-difluorophenyl)-6-azaspiro[3.4]octan-6-yl)(3-hydroxyisoxazol-5-yl)methanone;
(R)-(8-(2,4-difluorophenyl)-6-azaspiro[3.4]octan-6-yl)(3-hydroxyisoxazol-5-yl)methanone;
(S)-6-(8-(2,3-difluorophenyl)-6-azaspiro[3.4]octane-6-carbonyl)pyrazin-2(1H)-one;
(R)-6-(8-(2,3-difluorophenyl)-6-azaspiro[3.4]octane-6-carbonyl)pyrazin-2(1H)-one;
(S)-3-(8-(2,3-difluorophenyl)-6-azaspiro[3.4]octane-6-carbonyl)-1,2,4-oxadiazol-5(4H)-one;
(R)-3-(8-(2,3-difluorophenyl)-6-azaspiro[3.4]octane-6-carbonyl)-1,2,4-oxadiazol-5(4H)-one;
(S)-6-(8-(3,4-difluorophenyl)-6-azaspiro[3.4]octane-6-carbonyl)pyrazin-2(1H)-one;
(R)-6-(8-(3,4-difluorophenyl)-6-azaspiro[3.4]octane-6-carbonyl)pyrazin-2(1H)-one;
(S)-(8-(3,4-difluorophenyl)-6-azaspiro[3.4]octan-6-yl)(3-hydroxyisoxazol-5-yl)methanone;
(R)-(8-(3,4-difluorophenyl)-6-azaspiro[3.4]octan-6-yl)(3-hydroxyisoxazol-5-yl)methanone;
(S)-3-(8-(3,4-difluorophenyl)-6-azaspiro[3.4]octane-6-carbonyl)-1,2,4-oxadiazol-5(4H)-one;
(R)-3-(8-(3,4-difluorophenyl)-6-azaspiro[3.4]octane-6-carbonyl)-1,2,4-oxadiazol-5(4H)-one
(S)-3-(8-(2,4-difluorophenyl)-6-azaspiro[3.4]octane-6-carbonyl)-1,2,4-oxadiazol-5(4H)-one;
(R)-3-(8-(2,4-difluorophenyl)-6-azaspiro[3.4]octane-6-carbonyl)-1,2,4-oxadiazol-5(4H)-one;
(S)-3-(8-(3-fluorophenyl)-6-azaspiro[3.4]octane-6-carbonyl)-1,2,4-oxadiazol-5(4H)-one; and
(R)-3-(8-(3-fluorophenyl)-6-azaspiro[3.4]octane-6-carbonyl)-1,2,4-oxadiazol-5(4H)-one.
   or a salt and/or solvate of any one thereof.

Clause 82. A pharmaceutical composition comprising the compound or pharmaceutically acceptable salt and/or solvate thereof according to any one of clauses 3 to 81, wherein the proviso of formula (I) does not apply.

Clause 83. The compound or pharmaceutically acceptable salt and/or solvate thereof according to any one of clauses 3 to 81, for use as a pharmaceutical, wherein the proviso of formula (I) does not apply.

Clause 84. The compound or pharmaceutically acceptable salt and/or solvate thereof according to any one of clauses 3 to 81, for use in the treatment or prophylaxis of a disease or disorder in which inhibition of mPTP provides a therapeutic or prophylactic effect, wherein the proviso of formula (I) does not apply.

Clause 85. Use of the compound or pharmaceutically acceptable salt and/or solvate thereof according to any one of clauses 3 to 81, in the manufacture of a medicament for the treatment or prophylaxis of a disease or disorder in which inhibition of mPTP provides a therapeutic or prophylactic effect, wherein the proviso of formula (I) does not apply.

Clause 86. A method of preventing or treating a disease or disorder in which inhibition of mPTP provides a therapeutic or prophylactic effect in a subject, which comprises administering to a subject in need thereof an effective amount of a compound or pharmaceutically acceptable salt and/or solvate thereof according to any one of clauses 3 to 81, wherein the proviso of formula (I) does not apply.

Clause 87. The compound or pharmaceutically acceptable salt and/or solvate thereof for use, use or method according to any one of clauses 3 to 86, wherein the disease or disorder is selected from degenerative or neurodegenerative diseases, disorders of the central nervous system, ischemia and re-perfusion injury, metabolic diseases, inflammatory or autoimmune diseases, diseases of aging and renal diseases.

Clause 88. The compound or pharmaceutically acceptable salt and/or solvate thereof for use, use or method according to any one of clauses 3 to 86, wherein the disease or disorder is selected from degenerative or neurodegenerative diseases, disorders of the central nervous system, ischemia and re-perfusion injury, metabolic diseases, inflammatory or autoimmune diseases, diseases of aging, renal diseases, hearing loss, a disease or disorder of the eye, Charcot-Marie-Tooth disease (CMT1a) and Leigh syndrome disease.

Clause 89. The compound or pharmaceutically acceptable salt and/or solvate thereof for use, use or method according to clause 88, wherein the disease or disorder is a degenerative or neurodegenerative disease, such as Parkinson's disease, dementia with Lewy Bodies, Alzheimer's disease, amyotrophic lateral sclerosis, multiple sclerosis, frontal temporal dementia, chemotherapy induced neuropathy, Huntington's disease, spinocerebellar ataxias, progressive supranuclear palsy, hereditary spastic paraplegia, Duchenne muscular dystrophy, congenital muscular dystrophy, traumatic brain injury (such as concussion) and Friedreich's ataxia.

Clause 90. The compound or pharmaceutically acceptable salt and/or solvate thereof for use, use or method according to clause 88, wherein the disease or disorder is a disease of the central nervous system, such as AIDS dementia complex, depressive disorders, schizophrenia and epilepsy.

Clause 91. The compound or pharmaceutically acceptable salt and/or solvate thereof for use, use or method according to clause 88, wherein the disease or disorder is ischemia or re-perfusion injury, such as acute myocardial infarction, stroke, kidney ischemia reperfusion injury, and organ damage during transplantation.

Clause 92. The compound or pharmaceutically acceptable salt and/or solvate thereof for use, use or method according to clause 88, wherein the disease or disorder is a metabolic disease, such as hepatic steatosis, diabetes, diabetic retinopathy, cognitive decline and other diabetes associated conditions, obesity and feeding behaviours, and non-alcoholic fatty liver disease.

Clause 93. The compound or pharmaceutically acceptable salt and/or solvate thereof for use, use or method according to clause 88, wherein the disease or disorder is a metabolic disease, such as hepatic steatosis, diabetes, diabetic retinopathy, cognitive decline and other diabetes associated conditions, obesity and feeding behaviours, diabetic neuropathy and non-alcoholic fatty liver disease.

Clause 94. The compound or pharmaceutically acceptable salt and/or solvate thereof for use, use or method according to clause 88, wherein the disease or disorder is an inflammatory or autoimmune disease, such as acute pancreatitis, systemic lupus, organ failure in sepsis and hepatitis.

Clause 95. The compound or pharmaceutically acceptable salt and/or solvate thereof for use, use or method according to clause 88, wherein the disease or disorder is a disease of aging, such as bone repair, bone weakness in aging in osteoporosis and sarcopenia.

Clause 96. The compound or pharmaceutically acceptable salt and/or solvate thereof for use, use or method according to clause 88, wherein the disease or disorder is a renal disease, such as chronic kidney disease associated with APOL1 genetic variants and chronic kidney disease.

Clause 97. The compound or pharmaceutically acceptable salt and/or solvate thereof for use, use or method according to clause 88, wherein the disease or disorder is hearing loss, such as hearing loss due to aging, noise, concussion, traumatic brain injury (TBI), drug induced, and/or genetic hearing loss, including spinal muscular atrophy (SMA) syndrome (SMA1, SMA2, SMA3, and SMA4, also called Type I, II, III and IV).

Clause 98. The compound or pharmaceutically acceptable salt and/or solvate thereof for use, use or method according to clause 88, wherein the disease or disorder is a disease or disorder of the eye, such as age-related macular degeneration.

Clause 99. The compound or pharmaceutically acceptable salt and/or solvate thereof for use, use or method according to clause 88, wherein the disease or disorder is Charcot-Marie-Tooth disease (CMT1a).

Clause 100. The compound or pharmaceutically acceptable salt and/or solvate thereof for use, use or method according to clause 88, wherein the disease or disorder is Leigh syndrome disease.

Clause 101. The compound or pharmaceutically acceptable salt and/or solvate thereof according to any one of clauses 3 to 81, for use in the treatment or prophylaxis of a mitochondrial disease, wherein the proviso of formula (I) does not apply.

Clause 102. The compound or pharmaceutically acceptable salt and/or solvate thereof according to any one of clauses 3 to 81, for use in the treatment or prophylaxis of a disease or disorder associated with TDP-43 proteinopathy such as TDP-43 associated neurodegeneration, wherein the proviso of formula (I) does not apply.

Clause 103. Use of a compound of formula (I) or a pharmaceutically acceptable salt and/or solvate thereof according to any one of clauses 3 to 81, in the manufacture of a medicament for the treatment or prophylaxis of a disease or disorder associated with TDP-43 proteinopathy such as TDP-43 associated neurodegeneration, wherein the proviso of formula (I) does not apply.

Clause 104. A method of treating or preventing a disease or disorder associated with TDP-43 proteinopathy such as TDP-43 associated neurodegeneration, which comprises administering to a subject in need thereof an effective amount of a compound or pharmaceutically acceptable salt and/or solvate thereof according to any one of clauses 3 to 81, wherein the proviso of formula (I) does not apply.

Clause 105. The compound or pharmaceutically acceptable salt and/or solvate for use, use or method according to any one of clauses 102 to 104, wherein the disease or disorder is selected from Amyotrophic Lateral Sclerosis, Frontotemporal dementia, Facial onset sensory and motor neuronopathy, Primary lateral sclerosis, Progressive muscular atrophy, Inclusion body myopathy associated with early-onset Paget disease of the bone and Frontotemporal lobar degeneration dementia, Perry disease, Chronic traumatic encephalopathy, Severe traumatic brain injury, Alzheimer's disease, Hippocampal sclerosis dementia, Limbic-predominant age-related TDP-43 encephalopathy, and Cerebral age-related TDP-43 with sclerosis.

Clause 106. The compound or pharmaceutically acceptable salt and/or solvate thereof according to any one of clauses 3 to 81, for use in the treatment or prophylaxis of a disease or disorder associated with fibrosis, wherein the proviso of formula (I) does not apply.

Clause 107. Use of a compound or pharmaceutically acceptable salt and/or solvate thereof according to any one of clauses 3 to 81, in the manufacture of a medicament for the treatment or prophylaxis of a disease or disorder associated with fibrosis, wherein the proviso of formula (I) does not apply.

Clause 108. A method of treating or preventing a disease or disorder associated with fibrosis, which comprises administering to a subject in need thereof an effective amount of a compound or pharmaceutically acceptable salt and/or solvate thereof according to any one of clauses 3 to 81, wherein the proviso of formula (I) does not apply.

Clause 109. The compound or pharmaceutically acceptable salt and/or solvate thereof for use, use or method according to any one of clauses 106 to 108, wherein the disease or disorder is selected from chronic kidney disease, idiopathic pulmonary fibrosis, non-alcoholic steatohepatitis, primary biliary cholangitis and systemic sclerosis.

Clause 110. A process for the preparation of a compound of formula (I) according to clause 1, 2 or any one of clauses 8 to 76, which comprises reacting a compound of formula compound of formula (IIB),

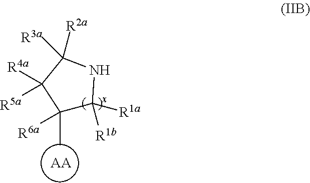

wherein R^{1a}, R^{1b}, R^{2a}, R^{3a}, R^{4a}, R^{5a}, R^{6a}, AA and x are as defined for the compound of formula (I), or a salt thereof,
with a compound of formula (IIIB),

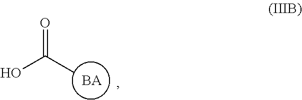

wherein BA is as defined for the compound of formula (I) or a salt thereof.

Clause 111. A compound selected from the group consisting of:
a compound of formula (IIB):

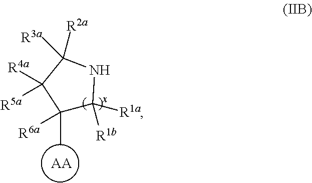

wherein R^{1a}, R^{1b}, R^{2a}, R^{3a}, R^{4a}, R^{5a}, R^{6a}, x and AA are as defined in any preceding clause
a compound of formula (IIIB):

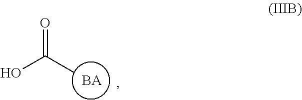

wherein BA is as defined in any preceding clause;
a compound of formula (IVB):

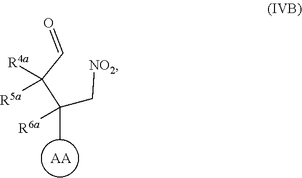

wherein R^{4a}, R^{5a}, R^{6a} and AA are as defined in any preceding clause;
a compound of formula (VIIB):

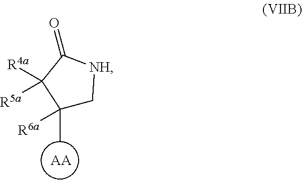

wherein R^{4a}, R^{5a}, R^{6a} and AA are as defined in any preceding clause;
a compound of formula (VIIIB):

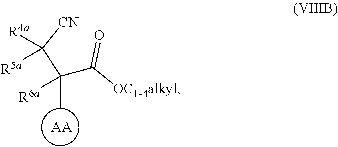

wherein R^{4a}, R^{5a}, R^{6a} and AA are as defined in any preceding clause;
a compound of formula (XIB):

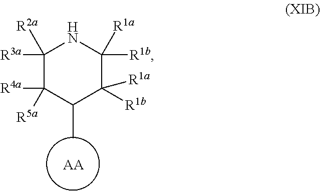

wherein R^{1a}, R^{2a}, R^{1a/b}, R^{2a}, R^{3a}, R^{4a}, R^{5a} and AA are as defined in any preceding clause;
a compound of formula (XIIB):

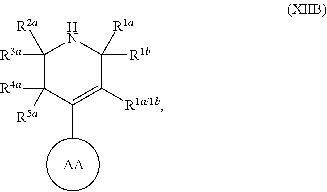

wherein R^{1a}, R^{2a}, R^{1a/b}, R^{2a}, R^{3a}, R^{4a}, R^{5a} and AA are as defined in any preceding clause;
a compound of formula (XIIIB):

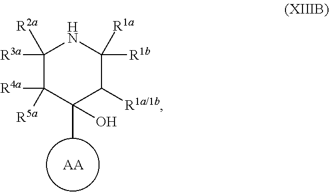

wherein R^{1a}, R^{2a}, R^{1a/b}, R^{2a}, R^{3a}, R^{4a}, R^{5a} and AA are as defined in any preceding clause; and
a compound of formula (XIVB):

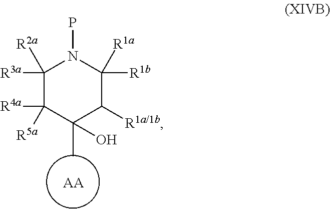

wherein R^{1a}, R^{2a}, R^{1a/b}, R^{2a}, R^{3a}, R^{4a}, R^{5a} and AA are as defined in any preceding clause, and P is a nitrogen protecting group such as BOC (tert-butyloxycarbonyl),
or salts, such as pharmaceutically acceptable salts, of any one thereof.

The invention is further exemplified by the following non-limiting examples.

### EXAMPLES

The invention is illustrated by the compounds described below. The following examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. The invention is meant to include such equivalents.

### General Experimental Details

Starting materials, reagents and solvents were obtained from commercial suppliers and used without further purification unless otherwise stated. Unless otherwise stated, all compounds with chiral centres are racemic. Where reactions are described as having been carried out in a similar manner to earlier, more completely described reactions, the general reaction conditions used were essentially the same. Work up conditions used were of the types standard in the art, but may have been adapted from one reaction to another. The starting material may not necessarily have been prepared from the batch referred to. Compounds synthesised may have various purities, ranging from for example 85% to 99%. Calculations of number of moles and yield are in some cases adjusted for this.

Purity of final compounds was confirmed by HPLC/MS analysis and determined to be at least ≥90%, and in the significant majority of cases ≥95%. Analytical LCMS was conducted using the instrumentation shown in Table 1 or Table 2. ¹H NMR were recorded at 300K in a Bruker 300 MHz instruments (ADVANCE III and ADVANCE III HD) or a Bruker 400 MHz (Avance Neo 400).

TABLE 1
Analytical LC-MS conditions

| Instrument | | | |
|---|---|---|---|
| ID | Column | Mobile Phase | Flow Rate |
| LCMS01 | Halo-C18, | A: H₂O/0.05% TFA; | 1.5 mL/min |
| | 30*3.0 mm, | B: ACN | |
| | 2.0 μm | | |
| LCMS02 | Cortecs C18+, | A: H₂O/0.05% TFA; | 1.5 mL/min |
| | 50*3.0 mm, | B: ACN/0.05% TFA | |
| | 2.7 μm | | |
| LCMS03 | Halo-C18, | A: H₂O/0.1% TFA; | 1.5 mL/min |
| | 30*3.0 mm, | B: ACN/0.05% FA | |
| | 2.0 μm | | |
| LCMS04 | Kinetex XB- | A: H₂O/0.01% TFA; | 1.5 mL/min |
| | C18, | B: ACN/0.01% FA | |
| | 50*3.0 mm, | | |
| | 2.6 μm | | |
| LCMS05 | Poroshell HPH- | A: H₂O/5 mM NH₄HCO₃; | 1.0 mL/min |
| | C18, | B: MeOH | |
| | 50*3.0 mm, | | |
| | 2.7 μm | | |
| LCMS06 | Xbridge C18, | A: H₂O/5 mM NH₄HCO₃ + | 1.2 mL/min |
| | 50*3.0 mm, | 0.05% NH₃•H₂O; | |
| | 3.5 μm | B: 5% H₂O in ACN | |
| LCMS07 | Poroshell HPH- | A: H₂O/0.05% NH₃•H₂O; | 1.2 mL/min |
| | C18, | B: ACN | |
| | 50*3.0 mm, | | |
| | 2.7 μm | | |
| LCMS08 | Halo C18, | A: H₂O/0.05% TFA; | 1.5 mL/min |
| | 50*3.0 mm, | B: ACN | |
| | 2.7 μm | | |
| LCMS09 | Poroshell HPH- | A: H₂O/0.05% NH₃•H₂O; | 1.2 mL/min |
| | C18, | B: ACN | |
| | 50*3.0 mm, | | |
| | 2.7 μm | | |

TABLE 2
Analytical LC-MS conditions

| Instrument | | | |
|---|---|---|---|
| ID | Column | Mobile Phase | Flow Rate |
| LCMS01 | Halo-C18, | A: 0.05% aqueous TFA; | 1.5 |
| | 30*3.0 mm, | B: ACN | mL/min |
| | 2.0 μm | | |
| LCMS02 | Cortecs C18+, | A: 0.05% aqueous TFA; | 1.5 |
| | 50*3.0 mm, | B: ACN/0.05% TFA | mL/min |
| | 2.7 μm | | |
| LCMS03 | Halo-C18, | A: 0.1% aqueous TFA; | 1.5 |
| | 30*3.0 mm, | B: ACN/0.05% FA | mL/min |
| | 2.0 μm | | |
| LCMS04 | Kinetex | A: 0.01% aqueous TFA; | 1.5 |
| | XB-C18, | B: ACN/0.01% FA | mL/min |
| | 50*3.0 mm, | | |
| | 2.6 μm | | |
| LCMS05 | Poroshell | A: 5 mM aqueous NH₄HCO₃; | 1.0 |
| | HPH-C18, | B: MeOH | mL/min |
| | 50*3.0 mm, | | |
| | 2.7 μm | | |
| LCMS06 | Xbridge C18, | A: 5 mM aqueous NH₄HCO₃ + | 1.2 |
| | 50*3.0 mm, | 0.05% NH₃•H₂O; | mL/min |
| | 3.5 μm | B: 5% H₂O in ACN | |
| LCMS07 | Poroshell | A: 0.05% aqueous NH₃; | 1.2 |
| | HPH-C18, | B: ACN | mL/min |
| | 50*3.0 mm, | | |
| | 2.7 μm | | |
| LCMS08 | Halo C18, | A: 0.05% aqueous TFA; | 1.5 |
| | 50*3.0 mm, | B: ACN | mL/min |
| | 2.7 μm | | |
| LCMS09 | Poroshell | A: 0.05% aqueous NH₃; | 1.2 |
| | HPH-C18, | B: ACN | mL/min |
| | 50*3.0 mm, | | |
| | 2.7 μm | | |
| LCMS10 | ZORBAX | A: 0.05% aqueous TFA; | 1.5 |
| | SB-Aq, | B: ACN | mL/min |
| | 50*4.6 mm, | | |
| | 1.8 um | | |
| LCMS11 | Halo-Aq C18, | A: 0.05% aqueous TFA; | 1.5 |
| | 30*3.0 mm, | B: ACN/0.05% TFA | mL/min |
| | 2.0 μm | | |
| LCMS12 | EVO C18 | A: 0.05% aqueous NH₃; | 1.2 |
| | 50*3.0 mm, | B: ACN | mL/min |
| | 2.6 um | | |
| LCMS13 | ZORBAX | A: 0.05% aqueous TFA; | 1.5 |
| | SB-Aq, | B: ACN/0.05% TFA | mL/min |
| | 50*4.6 mm, | | |
| | 1.8 um | | |
| LCMS14 | Cortecs C18+, | A: 0.05% aqueous TFA; | 1.5 |
| | 30*3.0 mm, | B: ACN | mL/min |
| | 2.7 μm | | |

### SYNTHESIS OF EXAMPLE COMPOUNDS

### Abbreviations

AcOH Acetic acid
ADP Adenosine-5′-Diphosphate
BOC₂O Di-tert-butyl dicarbonate
BOO tert-Butyloxycarbonyl
BSA Bovine serum albumin
CDI Carbonyldiimidazole
CH₃CN Acetonitrile
Cs₂CO₃ Caesium Carbonate
DAST Diethylaminosulfur trifluoride
DBU Diazabicyclo(5.4.0)undec-7-ene
DCM Dichloromethane
DIPEA Diisopropylethylamine
DMF Dimethylformamide
DMSO Dimethylsulfoxide
DPBS Dulbecco's Phosphate Buffered Saline
EDC 1-Ethyl-3-(3-dimethylaminopropyl)carbodiimide
EDTA Ethylenediamine tetraacetic acid
EGTA Ethylene glycol tetraacetic acid
Et₃N Triethylamine
EtOAc Ethyl acetate
EtOH Ethyl alcohol
ES Electrospray
FA Formic acid
HATU 1-[Bis(dimethylamino)methylene]-1H-1,2,3-triazolo[4,5-b]pyridinium 3-oxide hexafluorophosphate
HCl Hydrochloric acid
HEPES 4-(2-Hydroxyethyl)-1-piperazineethanesulfonic acid
HOBt Hydroxybenzotriazole
HPLC High Performance Liquid Chromatography
K₂CO₃ Potassium Carbonate
K₃PO₄ Potassium Phosphate
LAH Lithium aluminium hydride
LC-MS Liquid Chromatography-Mass Spectrometry
LDA Lithium diisopropylamine
LiHMDS Lithium bis(trimethylsilyl)amide
m Multiplet
m/z Mass-to-charge ratio
M Molar concentration
mg Milligram
MgSO₄ Magnesium sulfate
MeCN Acetonitrile
MeOH Methanol
MHz Megahertz
mmol Millimole
MOPS 3-(N-morpholino)propanesulfonic acid
MSA Methanesulfonic acid
NaHCO₃ Sodium bicarbonate
NaOAc Sodium acetate
NaOH Sodium hydroxide
Na₂SO₄ Sodium sulfate
NBS N-Bromosuccinimide
NCS N-Chlorosuccinimide
NH₄Cl Ammonium chloride
nm Nanometre
NMI N-Methylimidazole
NMR Nuclear Magnetic Resonance
PE Petroleum ether
Pd(OAc)₂ Palladium(II) Acetate
Pd(dppf)Cl₂·CH₂Cl₂ Bis(diphenylphosphino)ferrocene]dichloropalladium(II), complex with
dichloromethane
Pd(dppf)Cl₂ Bis(diphenylphosphino)ferrocene]dichloropalladium(II)
ppm parts per million
PTSA p-Toluenesulfonic acid
PyBOP Benzotriazol-1-yloxytripyrrolidinophosphonium hexafluorophosphate
RT Room temperature
o/n Overnight (16h)
SFC Supercritical fluid chromatography
TBAF tetra-n-Butylammonium fluoride
TBD Triazabicyclodecene
TCFH N′-Tetramethylformamidinium hexafluorophosphate
TEA Triethylamine
TFA Trifluoroacetic acid
THF Tetrahydrofuran
THP Tetrahydropyranyl
T₃P Propanephosphonic anhydride
TMSOTf Trimethylsilyl trifluoromethanesulfonate

### Intermediate 1: 1-methyl-5-oxo-4H-1,2,4-triazole-3-carboxylic acid

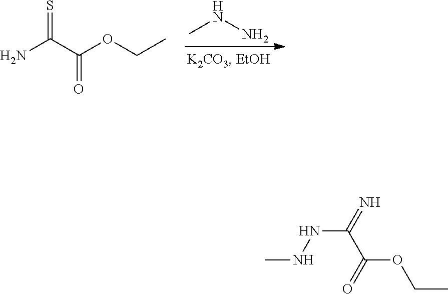

Into a 1 L round-bottom flask was added methyl hydrazine (35 g, 759.66 mmol, 1.00 equiv), K₂CO₃ (209.98 g, 1519.33 mmol, 2.00 equiv), EtOH (350 mL) and ethyl 2-amino-2-thioxoacetate (101.16 g, 759.66 mmol, 1.00 equiv) at room temperature. The mixture was stirred overnight and the resulting mixture filtered and the filter cake washed with EtOH (3×20 mL). The filtrate was concentrated under reduced pressure and the residue purified by silica gel column chromatography, eluting with PE/EtOAc (1:1) to afford ethyl 2-imino-2-(2-methylhydrazineyl)acetate (7 g) as a yellow oil.

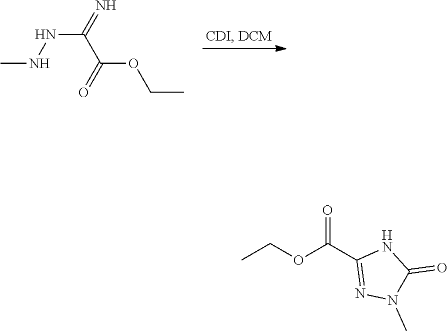

Into a 500 mL round-bottom flask was added ethyl 2-imino-2-(2-methylhydrazineyl)acetate (7 g, 48.22 mmol, 1.00 equiv), DCM (150 mL) and CDI (23.46 g, 144.67 mmol, 3.00 equiv) at room temperature. The mixture was stirred overnight and the resulting mixture was concentrated under reduced pressure. The residue was purified by reverse flash chromatography. This resulted in ethyl 1-methyl-5-oxo-4H-1,2,4-triazole-3-carboxylate (4.5 g, 54% yield) as a light yellow solid.

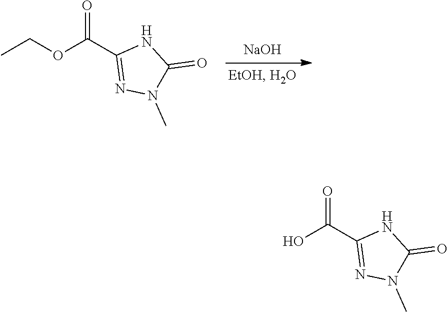

Into a 250 mL round-bottom flask was added ethyl 1-methyl-5-oxo-4H-1,2,4-triazole-3-carboxylate (2.5 g, 14.61 mmol, 1.00 equiv), EtOH (100 mL) and NaOH (1.75 g, 43.82 mmol, 3.00 equiv) at temperature. The mixture was stirred overnight at RT. The mixture as acidified to 5 with conc. HCl. The resulting mixture was concentrated under reduced pressure. The residue was purified by reverse flash chromatography to afford 1-methyl-5-oxo-4H-1,2,4-triazole-3-carboxylic acid (1.7 g, 81% yield) as a white solid.

LC-MS (ES, m/z): [M−H]⁻=142

### Example 1(a): (5-hydroxypyridin-3-yl)(8-phenyl-6-azaspiro[3.4]octan-6-yl)methanone

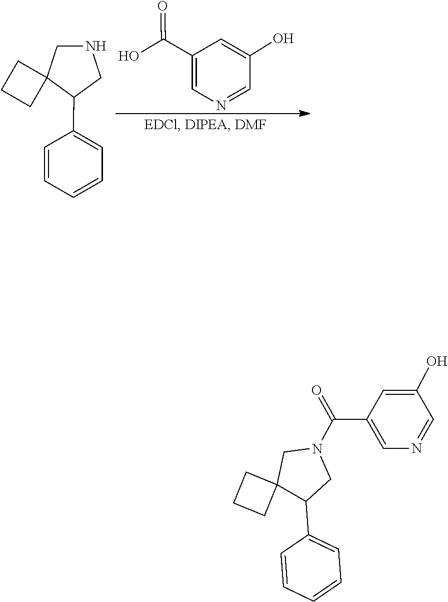

Into a 8-mL sealed tube, was placed 8-phenyl-6-azaspiro[3.4]octane (70.00 mg, 0.374 mmol, 1.00 equiv), 5-methoxypyridine-3-carboxylic acid (57.24 mg, 0.374 mmol, 1.00 equiv), DIPEA (120.62 mg, 0.935 mmol, 2.5 equiv), EDCl (107.48 mg, 0.561 mmol, 1.50 equiv), DMF (2.00 mL). The resulting solution was stirred for 6 hr at room temperature. The mixture was purified by Prep-HPLC. This resulted in 13 mg of 6-(5-methoxypyridine-3-carbonyl)-8-phenyl-6-azaspiro[3.4]octane as a white solid. LC-MS (ES, m/z): [M+H]⁺=309;

¹H-NMR (300 MHz, DMSO-d₆, ppm): 8.21-8.17 (m, 2H), 7.41-7.20 (m, 6H), 3.82-3.75 (m, 2H), 3.73-3.56 (m, 2H), 3.32-3.23 (m, 1H), 2.02-1.53 (m, 6H).

### Example 1(b) and 1(c): (5-hydroxypyridin-3-yl)(8-phenyl-6-azaspiro[3.4]octan-6-yl)methanone Enantiomer A and (5-hydroxypyridin-3-yl)(8-phenyl-6-azaspiro[3.4]octan-6-yl)methanone Enantiomer B

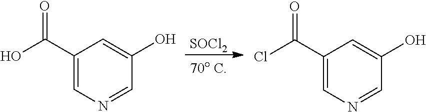

Into a 25-mL round-bottom flask, was placed 5-hydroxypyridine-3-carboxylic acid (220.00 mg, 1.58 mmol, 1.00 equiv), thionyl chloride (10 mL). The resulting solution was stirred for 12 h at 70° C. The reaction mixture was cooled to room temperature. The resulting mixture was concentrated. This resulted in 200 mg (80% yield) of 5-hydroxypyridine-3-carbonyl chloride as a light yellow solid.

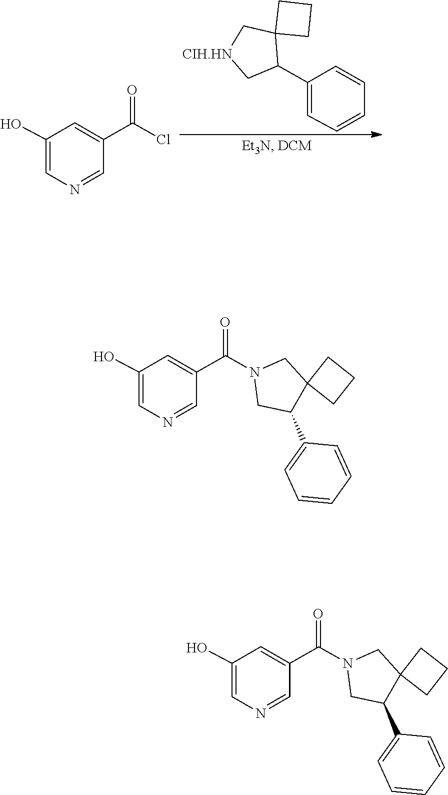

Into a 25-mL 3-necked round-bottom flask purged and maintained with an inert atmosphere of nitrogen, was placed 8-phenyl-6-azaspiro[3.4]octane hydrochloride (227.22 mg, 1.02 mmol, 1.00 equiv), DCM (10.00 mL), Et₃N (205.53 mg, 2.03 mmol, 2.00 equiv). This was followed by the addition of 5-hydroxypyridine-3-carbonyl chloride (160.00 mg, 1.02 mmol, 1.00 equiv) at 0° C. The resulting solution was stirred for 12 h at room temperature. The reaction was then quenched by the addition of 10 mL of water. The resulting solution was extracted with 2×15 mL of dichloromethane and the organic layer was dried over anhydrous sodium sulfate and concentrated. The residue was applied onto a silica gel column with ethyl acetate/petroleum ether (1/3). The racemic product was purified by Prep-SFC. This resulted in 30 mg of (5-hydroxypyridin-3-yl)(8-phenyl-6-azaspiro[3.4]octan-6-yl)methanone enantiomer A as an off-white solid. This resulted in 33 mg of (5-hydroxypyridin-3-yl)(8-phenyl-6-azaspiro[3.4]octan-6-yl)methanone enantiomer B as an off-white solid.

### Enantiomer A

LC-MS (ES, m/z): [M+H]⁺=309;

H-NMR (300 MHz, DMSO-d₆, ppm): 10.20 (brs, 1H), 8.24-8.22 (m, 2H), 7.40-7.22 (m, 6H), 3.83-3.57 (m, 4H), 3.28-3.24 (m, 1H), 2.03-1.49 (m, 6H).

### Enantiomer B

LC-MS (ES, m/z): [M+H]⁺=309;

H-NMR (300 MHz, DMSO-d₆, ppm): 10.20 (brs, 1H), 8.24-8.22 (m, 2H), 7.40-7.22 (m, 6H), 3.83-3.57 (m, 4H), 3.28-3.24 (m, 1H), 2.03-1.49 (m, 6H).

One of Enantiomers A and B is (S)-(5-hydroxypyridin-3-yl)(8-phenyl-6-azaspiro[3.4]octan-6-yl)methanone and the other is (R)-(5-hydroxypyridin-3-yl)(8-phenyl-6-azaspiro[3.4]octan-6-yl)methanone.

### Example 2: (6-hydroxypyrazin-2-yl)(8-phenyl-6-azaspiro[3.4]octan-6-yl)methanone

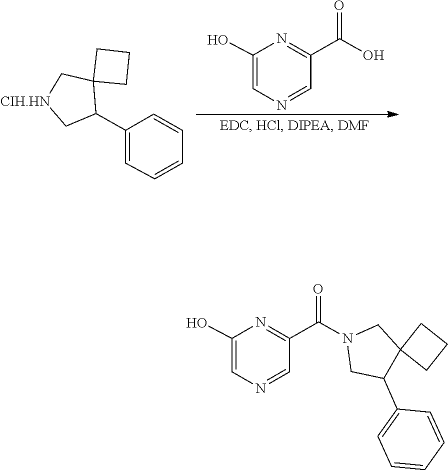

Into a 8-mL sealed tube, was placed 8-phenyl-6-azaspiro[3.4]octane hydrochloride (30.0 mg, 0.13 mmol, 1.00 eq), 6-hydroxypyrazine-2-carboxylic acid (20.66 mg, 0.15 mmol, 1.10 eq), EDC.HCl (38.56 mg, 0.20 mmol, 1.50 eq), DIPEA (41.93 mg, 0.325 mmol, 2.5 eq), DMF (2.00 mL). The resulting solution was stirred for 3 h at room temperature. The mixture was purified by Flash-Prep-HPLC. This resulted in 14 mg of 6-[8-phenyl-6-azaspiro[3.4]octane-6-carbonyl]pyrazin-2-ol as an off-white semi-solid. LC-MS (ES, m/z): [M+H]⁺=310;

H-NMR (300 MHz, DMSO-d₆, ppm): 8.26-8.18 (m, 2H), 7.40-7.21 (m, 5H), 3.99-3.67 (m, 4H), 3.30-3.24 (m, 1H), 2.03-1.51 (m, 6H).

### Example 3: (4-hydroxypyridin-2-yl)(8-phenyl-6-azaspiro[3.4]octan-6-yl)methanone

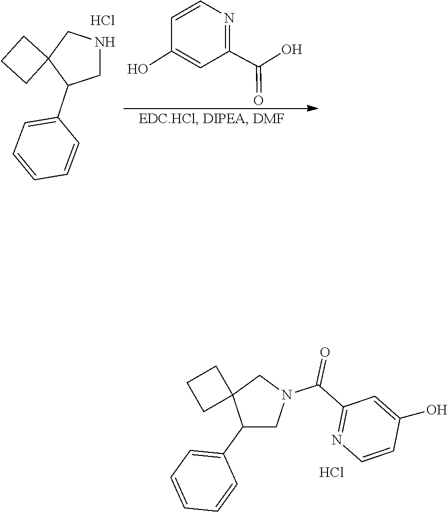

Into a 8-mL sealed tube, was placed 8-phenyl-6-azaspiro[3.4]octane hydrochloride (30.0 mg, 0.14 mmol, 1.00 eq), 4-hydroxypyridine-2-carboxylic acid (18.65 mg, 0.14 mmol, 1.00 eq), DMF (3.00 mL), EDC.HCl (38.56 mg, 0.20 mmol, 1.50 eq), DIPEA (45.15 mg, 0.35 mmol, 2.5 eq). The resulting solution was stirred for 2 h at room temperature. The mixture was purified by Flash-Prep-HPLC directly. This resulted in 16 mg of 2-[8-phenyl-6-azaspiro[3.4]octane-6-carbonyl]pyridin-4-ol as an off-white semi-solid. LC-MS (ES, m/z): [M+H]⁺=309; H-NMR (300 MHz, DMSO-d₆, ppm): 8.59-8.53 (m, 1H), 7.65 (s, 1H), 7.44-7.25 (m, 6H), 3.92-3.65 (m, 4H), 3.35-3.30 (m, 1H), 2.04-1.92 (m, 2H), 1.83-1.41 (m, 4H).

### Example 4: (3,3-dimethyl-4-phenylpyrrolidin-1-yl)(5-hydroxypyridin-3-yl)methanone

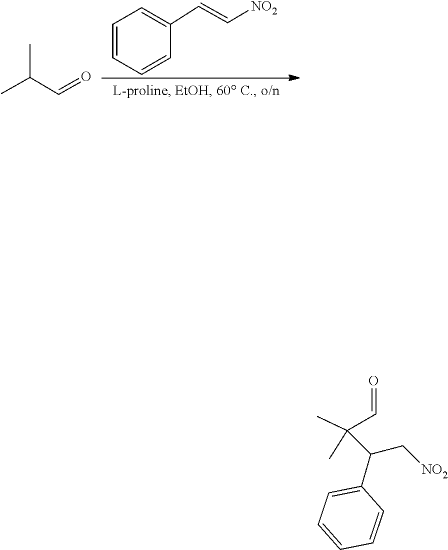

Into a 250-mL round-bottom flask purged and maintained with an inert atmosphere of nitrogen, was placed (E)-(2-nitrovinyl)benzene (0.70 g, 4.69 mmol, 1.00 equiv), isobutyraldehyde (1.02 g, 14.08 mmol, 3.00 equiv), EtOH (60 mL), L-proline (0.11 g, 0.96 mmol, 0.20 equiv). The resulting solution was stirred overnight at 60 degrees C. The reaction mixture was cooled to room temperature. The mixture was concentrated and the crude product was purified by Flash-Prep-HPLC. This resulted in 800 mg (77% yield) of 2,2-dimethyl-4-nitro-3-phenylbutanal as an off-white solid.

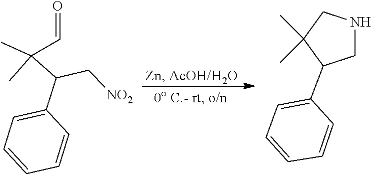 [SMILES: CC(C)(C=O)C(C[N+](=O)[O-])c1ccccc1 ; CC1(C)CNCC1c1ccccc1]

Into a 100-mL round-bottom flask purged and maintained with an inert atmosphere of nitrogen, was placed 2,2-dimethyl-4-nitro-3-phenylbutanal (420.00 mg, 1.90 mmol, 1.00 equiv), AcOH (20.00 mL), H₂O (20.00 mL). This was followed by the addition of Zn (372.49 mg, 5.70 mmol, 3.00 equiv) at 0 degrees C. The resulting solution was stirred overnight at room temperature. The solids were filtered out. The resulting mixture was concentrated. The crude product was purified by Flash-Prep-HPLC. This resulted in 150 mg (45% yield) of 3,3-dimethyl-4-phenylpyrrolidine as light yellow oil.

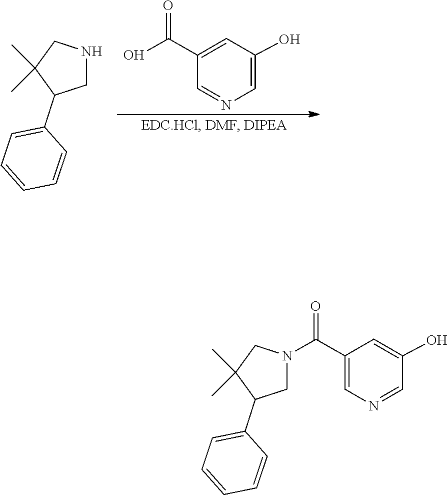 [SMILES: CC1(C)CNCC1c1ccccc1 ; O=C(O)c1cncc(O)c1 ; CC1(C)CN(C(=O)c2cncc(O)c2)CC1c1ccccc1]

Into an 8-mL sealed tube, was placed 3,3-dimethyl-4-phenylpyrrolidine (150.00 mg, 0.86 mmol, 1.00 equiv), 6-hydroxypyrazine-2-carboxylic acid (143.87 mg, 1.03 mmol, 1.20 equiv), EDC.HCl (246.09 mg, 1.28 mmol, 1.5 equiv), DIPEA (270.9 mg, 2.15 mmol, 2.50 equiv), DMF (4 mL). The resulting solution was stirred overnight at room temperature. The mixture was purified by Flash-Prep-HPLC. This resulted in 17.9 mg of 6-(3,3-dimethyl-4-phenylpyrrolidine-1-carbonyl)pyrazin-2-ol as an off-white solid. LC-MS (ES, m/z): [M+H]⁺=297;

H-NMR (300 MHz, DMSO-d₆, ppm): δ 10.21-10.16 (m, 1H), 8.31-8.20 (m, 2H), 7.39-7.22 (m, 6H), 4.06-3.70 (m, 2H), 3.54-3.11 (m, 3H), 1.07-0.98 (m, 3H), 0.81-0.66 (m, 3H).

### Example 5(a): 2-methyl-5-(8-phenyl-6-azaspiro[3.4]octane-6-carbonyl)-2,4-dihydro-3H-1,2,4-triazol-3-one

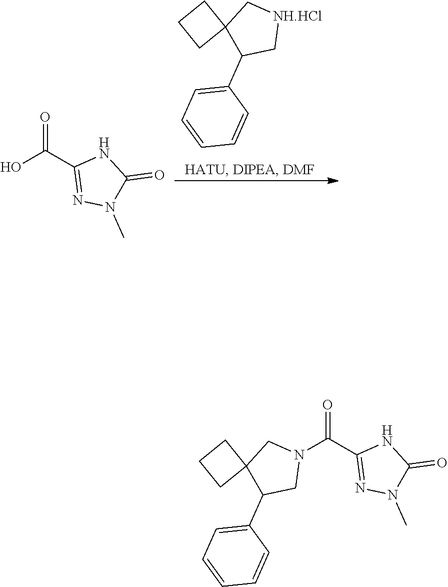

Into a 40-mL vial, was placed 1-methyl-5-oxo-4H-1,2,4-triazole-3-carboxylic acid (60.00 mg, 0.420 mmol, 1.00 equiv), DMF (3.00 mL), HATU (191.31 mg, 0.50 mmol, 1.20 equiv), DIPEA (108.38 mg, 0.84 mmol, 2.00 equiv), 8-phenyl-6-azaspiro[3.4]octane hydrochloride (93.81 mg, 0.420 mmol, 1.00 equiv). The resulting solution was stirred for 5 h at room temperature. The mixture was purified by Prep-HPLC. This resulted in 40 mg of 2-methyl-5-[8-phenyl-6-azaspiro[3.4]octane-6-carbonyl]-4H-1,2,4-triazol-3-one as an off-white solid. LC-MS (ES, m/z): [M+H]⁺=313;

H-NMR (300 MHz, DMSO-d₆, ppm): 12.19 (s, 1H), 7.38-7.22 (m, 5H), 4.19-3.6 (m, 4H), 3.31 (s, 3H), 3.23-3.27 (m, 1H), 2.00-1.49 (m, 6H).

### Example 5(b) and Example 5(c): 2-methyl-5-(8-phenyl-6-azaspiro[3.4]octane-6-carbonyl)-2,4-dihydro-3H-1,2,4-triazol-3-one Enantiomer A and 2-methyl-5-(8-phenyl-6-azaspiro[3.4]octane-6-carbonyl)-2,4-dihydro-3H-1,2,4-triazol-3-one Enantiomer B

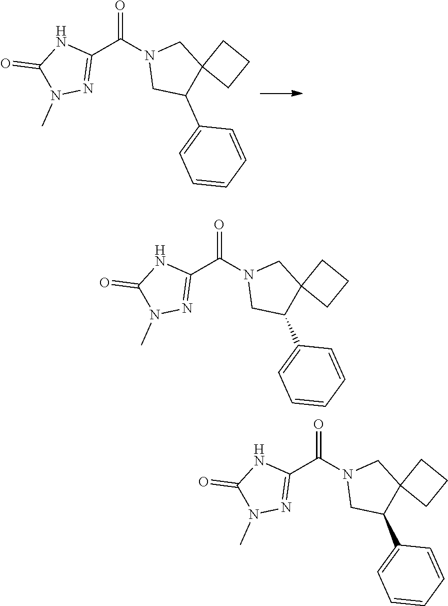

The enantiomers were separated by Prep-HPLC with the following conditions (Column: CHIRALCEL OJ-3, 50*4.6 mm, 3 um OJ3000-QK005; Mobile Phase: 70% EtOH (containing 0.2% MSA)/Hexane; Flow rate: 25 mL/min.; Wavelength: 254 nm, to give:

Ex. 5(b) Enantiomer A (Rt 5.5 min.) was isolated as an off-white solid, and assigned as 2-methyl-5-[(8S)-8-phenyl-6-azaspiro[3.4]octane-6-carbonyl]-4H-1,2,4-triazol-3-one (24 mg).

LCMS (ES, m/z): 313 [M+H]⁺, method LCMS2.

1H NMR (400 MHz, DMSO-d6, ppm) δ 11.96 (br, s, 1H), 7.40-7.35 (m, 2H), 7.32-7.20 (m, 3H), 4.24-4.05 (m, 1H), 3.97 (q, J=11.7 Hz, 1H), 3.83-3.79 (m, 1H), 3.67 (d, J=1.6 Hz, 1H), 3.39-3.35 (m, 3H), 3.30-3.26 (m, 1H), 1.99 (t, J=7.6 Hz, 2H), 1.91-1.69 (m, 2H), 1.71-1.44 (m, 2H).

Ex. 5(c) Enantiomer B (Rt 11.2 min.) was isolated as an off-white solid, and assigned as 2-methyl-5-[(8R)-8-phenyl-6-azaspiro[3.4]octane-6-carbonyl]-4H-1,2,4-triazol-3-one (26 mg).

LCMS (ES, m/z): 313 [M+H]⁺, method LCMS2.

1H NMR (400 MHz, DMSO-d6, ppm) δ 12.19 (br, s, 1H), 7.42-7.33 (m, 2H), 7.31-7.17 (m, 3H), 4.20-4.06 (m, 1H), 3.97 (q, J=11.8 Hz, 1H), 3.86-3.71 (m, 1H), 3.69-3.61 (m, 1H), 3.39-3.35 (m, 3H), 3.30-3.26 (m, 1H), 1.99 (t, J=7.6 Hz, 2H), 1.87-1.72 (m, 2H), 1.72-1.42 (m, 2H).

One of Enantiomers A and B is (S)-2-methyl-5-(8-phenyl-6-azaspiro[3.4]octane-6-carbonyl)-2,4-dihydro-3H-1,2,4-triazol-3-one and the other is (R)-2-methyl-5-(8-phenyl-6-azaspiro[3.4]octane-6-carbonyl)-2,4-dihydro-3H-1,2,4-triazol-3-one.

### Example 6a and 6b: (6-hydroxypyrazin-2-yl)(8-phenyl-6-azaspiro[3.4]octan-6-yl)methanone Enantiomer A and (6-hydroxypyrazin-2-yl)(8-phenyl-6-azaspiro[3.4]octan-6-yl)methanone Enantiomer B

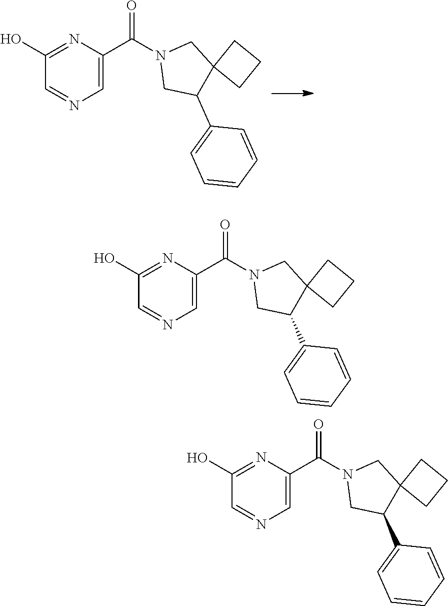

(6-hydroxypyrazin-2-yl)(8-phenyl-6-azaspiro[3.4]octan-6-yl)methanone (Example 65, 45 mg) was purified by Prep-SFC to afford (6-hydroxypyrazin-2-yl)(8-phenyl-6-azaspiro[3.4]octan-6-yl)methanone Enantiomer A (13 mg) as an off-white solid.

LC-MS Enantiomer A (ES, m/z): [M+H]⁺=310;

LC-MS Enantiomer B (ES, m/z): [M+H]⁺=310

H-NMR Enantiomer A (300 MHz, DMSO-d₆, ppm) δ 8.31-8.11 (m, 2H), 7.40-7.19 (m, 5H), 4.07-3.62 (m, 4H), 3.28-3.25 (m, 1H), 2.05-1.88 (m, 2H), 1.87-1.41 (m, 4H)

H-NMR Enantiomer B (300 MHz, DMSO-d₆, ppm) δ 8.31-8.11 (m, 2H), 7.40-7.19 (m, 5H), 4.07-3.62 (m, 4H), 3.28-3.25 (m, 1H), 2.05-1.88 (m, 2H), 1.87-1.41 (m, 4H) One of Enantiomers A and B is (S)-(6-hydroxypyrazin-2-yl)(8-phenyl-6-azaspiro[3.4]octan-6-yl)methanone and the other is (R)-(6-hydroxypyrazin-2-yl)(8-phenyl-6-azaspiro[3.4]octan-6-yl)methanone.

### Example 7: 6-(8-(2-fluorophenyl)-6-azaspiro[3.4]octane-6-carbonyl)pyrazin-2(1H)-one

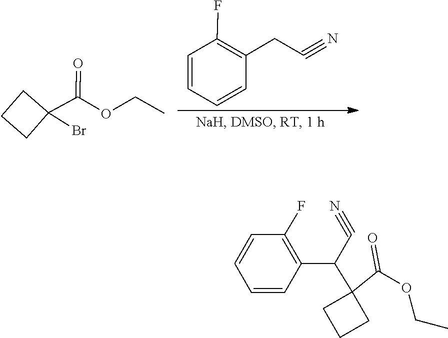

To a solution of benzeneacetonitrile, 2-fluoro-(3 g, 22.20 mmol, 1.00 equiv) in DMSO(30 mL) was added sodium hydride (60% in oil, 1.33 g, 33.30 mmol, 1.50 equiv) at 10° C. The mixture was stirred for 15 min. ethyl 1-bromocyclobutane-1-carboxylate (5.06 g, 24.42 mmol, 1.10 equiv) was added and the mixture was allowed to warm to RT and stirred for 2 h. The reaction mixture was quenched by water and extracted with EtOAc (2×50 mL), dried over anhydrous Na₂SO₄. After filtration, the filtrate was concentrated under reduced pressure. The residue was purified by reverse phase flash to afford ethyl 1-[cyano(2-fluorophenyl)methyl]cyclobutane-1-carboxylate (2.8 g, 48% yield) as a light yellow oil.

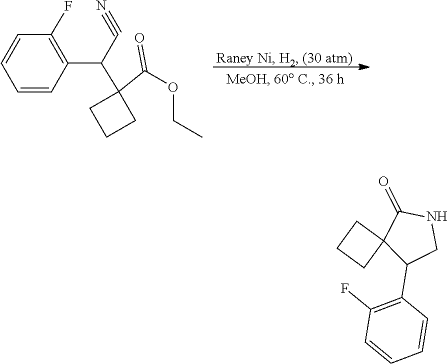 [SMILES: CCOC(=O)C1(C(C#N)c2ccccc2F)CCC1 ; O=C1NCC(c2ccccc2F)C12CCC2]

To a solution of ethyl 1-[cyano(2-fluorophenyl)methyl]cyclobutane-1-carboxylate (2.50 g, 9.57 mmol, 1.00 equiv) in 20 mL MeOH was added Raney Ni (0.25 g, 2.87 mmol, 0.30 equiv) in a pressure tank. The mixture was purged with nitrogen and then was pressurized to 30 atm with hydrogen at 60° C. for 18 h. The reaction mixture was cooled to room temperature and filtered to remove insoluble solids. The resulting mixture was concentrated under reduced pressure. The crude product was purified by Prep. HPLC to afford 8-(2-fluorophenyl)-6-azaspiro[3.4]octan-5-one (1 g, 48% yield) as a light yellow oil.

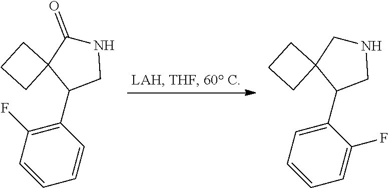 [SMILES: O=C1NCC(c2ccccc2F)C12CCC2 ; Fc1ccccc1C1CNCC12CCC2]

To a stirred solution of 8-(2-fluorophenyl)-6-azaspiro[3.4]octan-5-one (400 mg, 1.82 mmol, 1.00 equiv) in THF (10 mL) was added LAH (138.48 mg, 3.65 mmol, 2.00 equiv) in portions at 0° C. under nitrogen atmosphere. The resulting mixture was stirred for 5 h at 60° C. under nitrogen atmosphere. The mixture was allowed to cool down to room temperature. The reaction was quenched by the addition of 15%. NaOH (aq.) (0.1 mL) at 0° C. The resulting mixture was filtered; the filter cake was washed with THF (2×10 mL). The filtrate was concentrated under reduced pressure. The residue was purified by reverse phase flash to afford 8-(2-fluorophenyl)-6-azaspiro[3.4]octane (230 mg, 61% yield) as a light yellow oil.

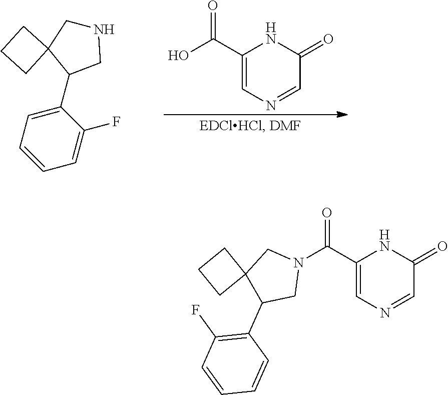 [SMILES: Fc1ccccc1C1CNCC12CCC2 ; O=C(O)c1cncc(=O)[nH]1 ; O=C(c1cncc(=O)[nH]1)N1CC(c2ccccc2F)C2(CCC2)C1]

Prepared as for Example 1, Step 3 using 6-oxo-1H-pyrazine-2-carboxylic acid (50 mg, 0.357 mmol, 1.00 equiv) and 8-(2-fluorophenyl)-6-azaspiro[3.4]octane (73.26 mg, 0.357 mmol, 1.00 equiv) to afford 6-[8-(2-fluorophenyl)-6-azaspiro[3.4]octane-6-carbonyl]-1H-pyrazin-2-one (15 mg) as an off-white solid. LC-MS (ES, m/z): [M+H]⁺=328; H-NMR (400 MHz, DMSO-d₆, ppm): 8.02-7.95 (m, 2H), 7.32-7.18 (m, 4H), 3.98-3.88 (m, 1H), 3.84-3.53 (m, 4H), 2.00-1.57 (m, 6H).

### Example 8: 6-(3,3-dimethyl-4-phenylpyrrolidine-1-carbonyl)pyrazin-2(1H)-one

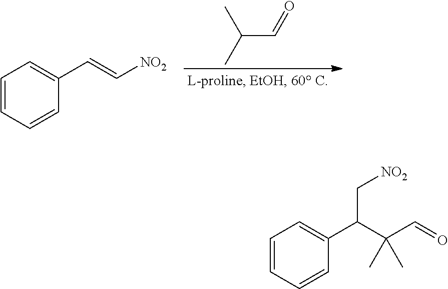

Into a 40 mL vial were added p-nitrostyrene (500 mg, 3.352 mmol, 1.00 equiv), EtOH (15 mL) isobutyraldehyde (483.46 mg, 6.704 mmol, 2.00 equiv) and L-proline (115.79 mg, 1.006 mmol, 0.30 equiv) at room temperature. The resulting mixture was stirred for 3 h at 60° C. under nitrogen atmosphere. The mixture was allowed to cool down to room temperature. The resulting mixture was concentrated under reduced pressure. The residue was purified by silica gel column chromatography, eluted with PE/EA (10:1) to afford 2,2-dimethyl-4-nitro-3-phenylbutanal (350 mg, 47% yield) as a light yellow oil.

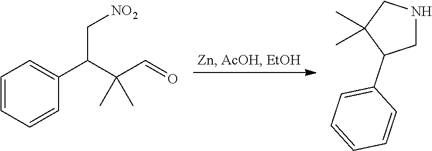 [SMILES: CC(C)(C=O)C(C[N+](=O)[O-])c1ccccc1 ; CC1(C)CNCC1c1ccccc1]

Into a 50 mL round-bottom flask were added 2,2-dimethyl-4-nitro-3-phenylbutanal (350 mg, 1.582 mmol, 1.00 equiv), EtOH/AcOH(10 mL/2 mL) and Zn (517.35 mg, 7.910 mmol, 5.00 equiv) at room temperature. The resulting mixture was stirred for 12 h at room temperature under nitrogen atmosphere. The resulting mixture was filtered, the filter cake was washed with EtOH 1×10 mL. The filtrate was concentrated under reduced pressure. The residue was basified to pH 10 with aq. NaOH (15%). The resulting mixture was extracted with EtOAc (2×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 3,3-dimethyl-4-phenylpyrrolidine (180 mg, 65% yield) as a colorless oil.

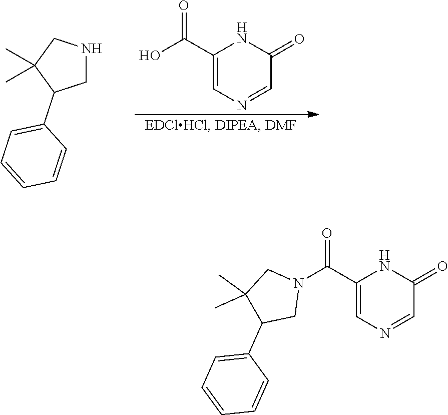 [SMILES: CC1(C)CNCC1c1ccccc1 ; O=C(O)c1cncc(=O)[nH]1 ; CC1(C)CN(C(=O)c2cncc(=O)[nH]2)CC1c1ccccc1]

Prepared as for Example 1 using 3,3-dimethyl-4-phenylpyrrolidine (70 mg, 0.399 mmol, 1.00 equiv), and 6-oxo-1H-pyrazine-2-carboxylic acid (55.95 mg, 0.399 mmol, 1.00 equiv) to afford 6-(3,3-dimethyl-4-phenylpyrrolidine-1-carbonyl)-1H-pyrazin-2-one (10.1 mg) as an off-white solid.

LC-MS (ES, m/z): [M+H]⁺=298

H-NMR (300 MHz, DMSO-d₆, ppm): 8.26-8.17 (m, 2H), 7.39-7.23 (m, 5H), 4.24-4.03 (m, 1H), 3.99-3.90 (m, 1H), 3.67-3.34 (m, 2H), 3.19-3.14 (m, 1H), 1.07-1.01 (m, 3H), 0.77-0.68 (m, 3H).

### Example 9: 6-(3,3-dimethyl-4-phenylpiperidine-1-carbonyl)pyrazin-2(1H)-one

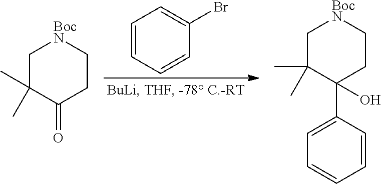

In a 50-mL round bottom flask, to a solution of bromobenzene (0.46 g, 2.93 mmol, 1 equiv) in THF (50 mL) was added dropwise n-butyllithium solution (20.0 M in THF/hexane, 2.94 mL, 5.87 mmol) at −78 degrees C. under N₂ atmosphere. The reaction mixture was stirred at −78 degrees C. for 30 mins. Then a solution of tert-butyl 3,3-dimethyl-4-oxopiperidine-1-carboxylate (1 g, 4.40 mmol, 1.5 equiv) in 50 mL THF was added dropwise and the mixture was stirred for another 30 mins. The reaction was quenched with water/sat. NH₄Cl (20 mL), and then the mixture was extracted with ether/EtOAc (2×15 mL). The combined organic extracts were washed with brine (10 mL), dried over anhydrous Na₂SO₄, and concentrated under vacuum to yield a crude product which was directly purified by eluted with PE/EA (10:1) to afford tert-butyl 4-hydroxy-3,3-dimethyl-4-phenylpiperidine-1-carboxylate (500 mg, 56%) as a light yellow solid.

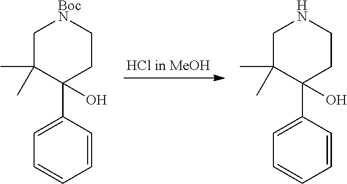 [SMILES: CC(C)(C)OC(=O)N1CCC(O)(c2ccccc2)C(C)(C)C1 ; CC1(C)CNCCC1(O)c1ccccc1]

Into a 50 mL 2-necked round-bottom flask were added tert-butyl 4-hydroxy-3,3-dimethyl-4-phenylpiperidine-1-carboxylate (400 mg, 1.31 mmol, 1 equiv) and HCl in MeOH (10 mL) at room temperature. The mixture was basified to pH 7 with saturated NaHCO₃ (aq.). The aqueous layer was extracted with EtOAc (2×20 mL). The resulting mixture was concentrated under reduced pressure to afford 3,3-dimethyl-4-phenylpiperidin-4-ol (250 mg, 93%) as a light yellow oil.

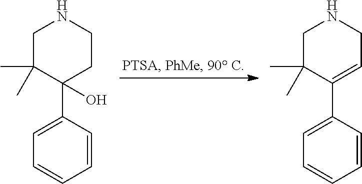

Into a 50 mL 2-necked round-bottom flask were added 3-methyl-4-phenylpiperidin-4-ol (230 mg, 1.20 mmol, 1 equiv) in Toluene (30 mL) and PTSA (310.60 mg, 1.80 mmol, 1.5 equiv) at 90° C. The reaction was quenched with water (1 mL), and then the mixture was extracted with ether/EtOAc (2×15 mL). The combined organic extracts were washed with brine (10 mL), dried over anhydrous Na₂SO₄, and concentrated under vacuum to yield a crude product which was directly purified by flash chromatography (PE/EA) mixture to yield to afford 3,3-dimethyl-4-phenyl-2,6-dihydro-1H-pyridine (200 mg, 89%) as an off-white oil.

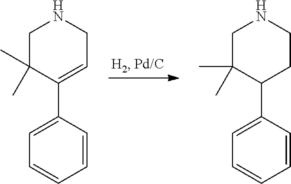

To a solution of 3,3-dimethyl-4-phenyl-2,6-dihydro-1H-pyridine (180 mg, 0.96 mmol, 1 equiv) in 10 mL MeOH was added Pd/C (50%, 85.23 mg) in a pressure tank. The mixture was hydrogenated at room temperature under 15 atm of hydrogen pressure for overnight, filtered through a Celite pad and concentrated under reduced pressure. The resulting mixture was concentrated under reduced pressure to afford 3,3-dimethyl-4-phenylpiperidine (150 mg, 82%) as an off-white solid.

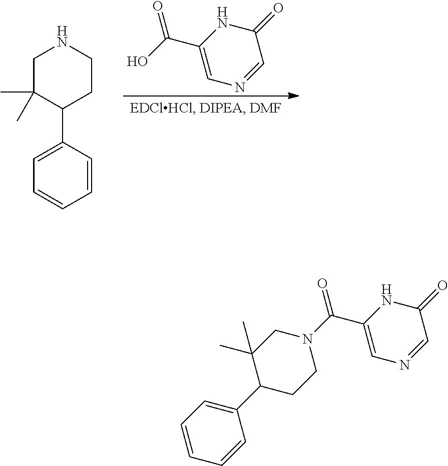 [SMILES: CC1(C)CNCCC1c1ccccc1 ; O=C(O)c1cncc(=O)[nH]1 ; CC1(C)CN(C(=O)c2cncc(=O)[nH]2)CCC1c1ccccc1]

Into a 8 mL vial were added 3,3-dimethyl-4-phenylpiperidine (40 mg, 0.21 mmol, 1 equiv) in DMF (4.00 mL), 6-oxo-1H-pyrazine-2-carboxylic acid (32.56 mg, 0.23 mmol, 1.1 equiv), HATU (88.38 mg, 0.23 mmol, 1.1 equiv), DIPEA (81.93 mg, 0.63 mmol, 3 equiv) at room temperature. Desired product could be detected by LCMS (254 nm, 24%). The residue was purified by reverse flash chromatography with the following conditions: column, silica gel; mobile phase, MeCN in water, 10% to 50% gradient in 10 min; detector, UV 254 nm to afford 6-(3,3-dimethyl-4-phenylpiperidine-1-carbonyl)-1H-pyrazin-2-one (20 mg, 30%) as an off-white solid. LCMS (ES, m/z): [M+H]⁺=312 H-NMR (300 MHz, DMSO-d₆, ppm): 8.11 (s, 1H), 7.97 (s, 1H), 7.31-7.16 (m, 5H), 3.97-3.70 (m, 3H), 2.71-2.65 (m, 1H), 2.20-2.15 (m, 1H), 1.56-1.51 (m, 1H), 0.76 (s, 6H).

### Example 10(a) and 10(b): 6-(3,3-dimethyl-4-phenylpyrrolidine-1-carbonyl)pyrazin-2(1H)-one Enantiomer A and 6-(3,3-dimethyl-4-phenylpyrrolidine-1-carbonyl)pyrazin-2(1H)-one Enantiomer B

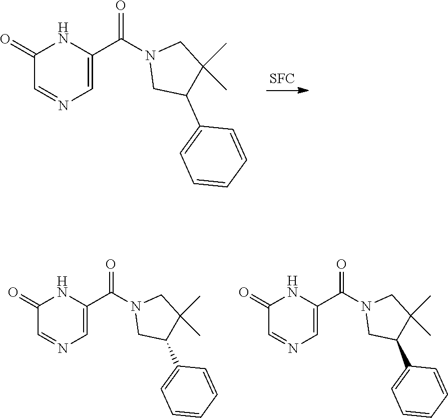

The 6-(-3,3-dimethyl-4-phenylpyrrolidine-1-carbonyl)-1H-pyrazin-2-one (Example 9, 15 mg) was purified by Chiral HPLC with the following conditions: YMC Cellulose-SB, 100×4.6 mm, 3 μm 121AB00077, mobile phase A, n-Hexane/DCM=3/1, mobile phase B Ethanol(0.1% FA), 10% to 50% gradient in 15 min; detector, UV 254 nm and 220 nm to afford 6-(3,3-dimethyl-4-phenylpyrrolidine-1-carbonyl)pyrazin-2(1H)-one (Enantiomer A) (5 mg, 33%) as an off-white solid and 6-(3,3-dimethyl-4-phenylpyrrolidine-1-carbonyl)pyrazin-2(1H)-one (Enantiomer B) (5 mg, 33%) as an off-white solid.

LCMS Enantiomer A (ES, m/z): [M+H]⁺=298;

LCMS Enantiomer B (ES, m/z): [M+H]⁺=298

H-NMR (Enantiomer A) (300 MHz, CDCl₃, ppm): δ 7.54 (s, 1H), 7.49-7.39 (m, 1H), 7.34-7.24 (m, 2H), 7.17-7.06 (m, 3H), 3.74-3.70 (m, 1H), 3.68 (d, 1H), 3.59-3.53 (m, 2H), 3.14-3.02 (m, 2H), 1.01 (s, 3H), 0.87 (s, 3H).

H-NMR (Enantiomer B) (300 MHz, DMSO-d6, ppm): δ 8.40-8.02 (m, 2H), 7.48-7.13 (m, 5H), 4.19-3.83 (m, 2H), 3.70-3.47 (m, 2H), 3.22-3.11 (m, 1H), 1.03 (d, J=20.4 Hz, 3H), 0.71 (d, J=30.9 Hz, 3H).

One of Enantiomers A and B is (S)-6-(3,3-dimethyl-4-phenylpyrrolidine-1-carbonyl)pyrazin-2(1H)-one and the other is (R)-6-(3,3-dimethyl-4-phenylpyrrolidine-1-carbonyl)pyrazin-2(1H)-one.

### Example 11: 5-(8-(2-fluorophenyl)-6-azaspiro[3.4]octane-6-carbonyl)-2-methyl-2,4-dihydro-3H-1,2,4-triazol-3-one

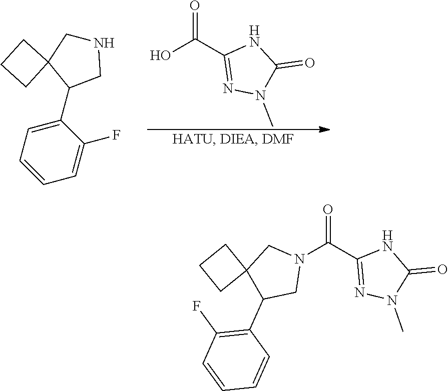

Prepared as for Example 5A using 8-(2-fluorophenyl)-6-azaspiro[3.4]octane (50 mg, 0.244 mmol, 1.00 equiv) and 1-methyl-5-oxo-4H-1,2,4-triazole-3-carboxylic acid (Intermediate 1, 34.86 mg, 0.244 mmol, 1.00 equiv) to give 5-[8-(2-fluorophenyl)-6-azaspiro[3.4]octane-6-carbonyl]-2-methyl-4H-1,2,4-triazol-3-one (22 mg, 27% yield) as an off-white solid. LC-MS-PH (ES, m/z): [M+H]⁺=331

H-NMR (400 MHz, DMSO-d₆, ppm): 11.56 (br, 1H), 7.33-7.14 (m, 4H), 4.20-4.04 (m, 2H), 3.90-3.50 (m, 3H), 3.38-3.33 (m, 3H), 2.00-1.99 (m, 2H), 1.94-1.60 (m, 4H).

H-NMR (400 MHz, DMSO-d₆, ppm) δ 12.33 (s, 1H), 8.27-8.21 (m, 1H), 7.22-7.20 (m, 2H), 7.02-7.00 (m, 1H), 6.93-6.88 (m, 1H), 3.83 (s, 3H), 3.37 (s, 3H), 3.18 (d, J=6.4 Hz, 2H), 2.72 (s, 2H), 1.81-1.73 (m, 6H).

The following Examples were prepared using analogous methods disclosed for Examples 1 to 11:

TABLE 3
Synthesis of certain Example compounds

| Example | | | | |
|---|---|---|---|---|
| No | Name | Structure | LCMS; m/z, Rt | 1H NMR |
| 12 | 6-(1-phenyl-2- azaspiro[3.3] heptane-2- carbonyl)pyrazin- 2(1H)-one | 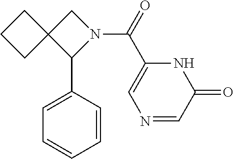 [SMILES: O=C(c1cncc(=O)[nH]1)N1CC2(CCC2)C1c1ccccc1] | [M + H]⁺ = 296; 1.2 min | (300 MHz, DMSO-d₆, ppm): 7.77 (s, 1H), 7.59 (s, 1H), 7.43-7.11 (m, 5H), 6.07 (br, 1H), 5.13 (s, 1H), 4.60-4.42 (m, 2H), 2.28- 2.08 (m, 2H), 1.72-1.39 (m, 4H) |
| 13 | 6-(3-methyl-4- phenylpyrrolidine- 1- carbonyl)pyrazin- 2(1H)-one | 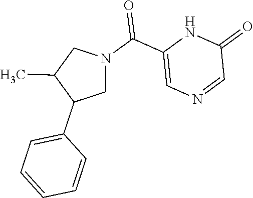 [SMILES: CC1CN(C(=O)c2cncc(=O)[nH]2)CC1c1ccccc1] | [M + H]⁺ = 284; 1.16 min | (300 MHz, DMSO-d₆, ppm): 8.24-8.15 (m, 2H), 7.37-7.23 (m, 5H), 4.20- 3.80 (m, 2H), 3.55-3.15 (m, 2H), 2.97-2.87 (m, 1H), 2.41-2.36 (m, 1H), 0.96-0.87 (m, 3H). |
| 50 | 2-methyl-5-(3- methyl-3- phenylpyrrolidine- 1-carbonyl)-2,4- dihydro-3H-1,2,4- triazol-3-one | 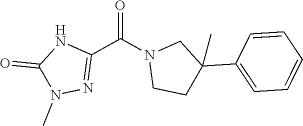 [SMILES: Cn1nc(C(=O)N2CCC(C)(c3ccccc3)C2)[nH]c1=O] | [M + H]⁺ = 287.1; 3.99 min | 1H-NMR (400 MHz, DMSO-D6) δ 8.30 (s, 1H), 7.31 (d, J = 3.7 Hz, 4H), 7.21-7.19 (m, 1H), 4.11- 3.88 (m, 2H), 3.79-3.52 (m, 2H), 2.95-2.78 (m, 3H), 2.22-2.03 (m, 2H), 1.29 (m, 3H) |
| 51 | 2-methyl-5-(4- phenylpiperidine- 1-carbonyl)-2,4- dihydro-3H-1,2,4- triazol-3-one | 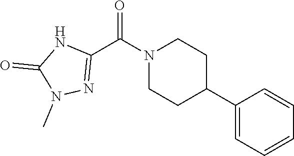 [SMILES: Cn1nc(C(=O)N2CCC(c3ccccc3)CC2)[nH]c1=O] | [M − H]⁻ = 287.1; 4.08 min | 1H-NMR (400 MHz, DMSO-D6) δ 7.32-7.18 (m, 5H), 4.91 (d, J = 13.7 Hz, 1H), 4.56 (d, J = 12.8 Hz, 1H), 3.34 (d, J = 4.1 Hz, 3H), 3.22 (t, J = 11.9 Hz, 1H), 2.89-2.81 (m, 2H), 1.86 (t, J = 9.8 Hz, 2H), 1.68-1.49 (m, 2H). |

### Example 14(a): 8-Phenyl-6-(3H-1,2,3-triazole-4-carbonyl)-6-azaspiro[3.4]octane Enantiomer A and 8-phenyl-6-(3H-1,2,3-triazole-4-carbonyl)-6-azaspiro[3.4]octane Enantiomer B

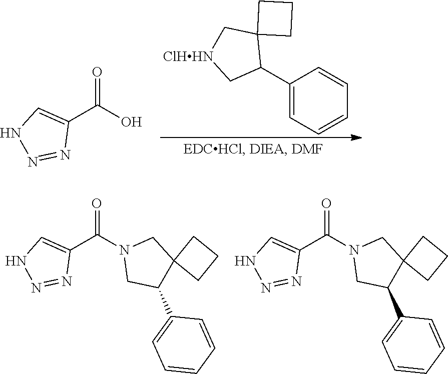

To a solution of 8-phenyl-6-azaspiro[3.4]octane hydrochloride (150 mg, 0.7 mmol) in DMF (1.5 mL) was added 3H-1,2,3-triazole-4-carboxylic acid (99 mg, 0.9 mmol, 1.3 eq), EDC.HCl (0.21 g, 1.1 mmol, 1.6 eq) and DIPEA (0.22 g, 1.7 mmol, 2.5 eq). The mixture was stirred for 2 h then quenched by H₂O (0.5 mL) and the resultant mixture purified directly by Prep-HPLC with the following conditions (Xselect CSH C18 Column, 30*150 mm, 5 μm, Mobile Phase: 25-75% ACN/0.05% aqueous HCl over 8 min., 1.50 L/min) to afford racemic 8-phenyl-6-(3H-1,2,3-triazole-4-carbonyl)-6-azaspiro[3.4]octane (80 mg) as an off-white solid.

The enantiomers were separated by Prep-HPLC with the following conditions (Column: CHIRALCEL OJ-H 2*25 cm, 5 μm; Mobile Phase: 50% EtOH (containing 0.2% MSA)/Hexane; Flow rate: 25 mL/min; Wavelength: 254 nm) to give: Ex. 14(a) Enantiomer A (Rt 12.6 min.) was isolated as an off-white solid, (24 mg, 32% yield).

LCMS (ES, m/z): 283 [M+H]⁺, method LCMS10.

¹H NMR (300 MHz, DMSO-d₆, ppm) δ 8.37 (br, s, 1H), 7.40-7.33 (m, 2H), 7.30-7.20 (m, 3H), 4.24-4.10 (m, 1H), 4.02 (s, 1H), 3.87-3.76 (m, 1H), 3.69 (s, 1H), 3.37-3.32 (m, 1H), 2.09-1.93 (m, 2H), 1.90-1.70 (m, 2H), 1.68-1.45 (m, 2H).

Ex. 14(b) Enantiomer B (Rt 7.7 min.) was isolated as an off-white solid, (16 mg, 21% yield).

LCMS (ES, m/z): 283 [M+H]⁺, method LCMS10.

¹H NMR (300 MHz, DMSO-d₆, ppm) δ 8.37 (br, s, 1H), 7.42-7.32 (m, 2H), 7.30-7.20 (m, 3H), 4.24-4.10 (m, 1H), 4.02 (s, 1H), 3.86-3.78 (m, 1H), 3.68 (s, 1H), 3.32-3.22 (m, 1H), 2.06-1.93 (m, 2H), 1.91-1.72 (m, 2H), 1.69-1.47 (m, 2H).

One of Enantiomers A and B is (S)-8-Phenyl-6-(3H-1,2,3-triazole-4-carbonyl)-6-azaspiro[3.4]octane and the other is (R)-8-Phenyl-6-(3H-1,2,3-triazole-4-carbonyl)-6-azaspiro[3.4]octane.

### Examples 15 (a)-(d): 6-(3-ethyl-4-phenylpyrrolidine-1-carbonyl)pyrazin-2(1H)-one Diastereomer A, 6-(3-ethyl-4-phenylpyrrolidine-1-carbonyl)pyrazin-2(1H)-one Diastereomer B, 6-(3-ethyl-4-phenylpyrrolidine-1-carbonyl)pyrazin-2(1H)-one Diastereomer C and 6-(3-ethyl-4-phenylpyrrolidine-1-carbonyl)pyrazin-2(1H)-one Diastereomer D

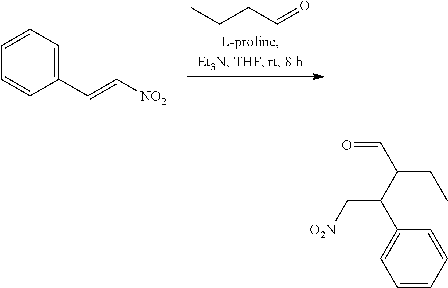

To a stirred solution of p-nitrostyrene (5.0 g, 34 mmol) and butanal (3.6 g, 50 mmol, 1.5 eq) in THF (40 mL) was added L-proline (1.2 g, 10 mmol, 0.3 eq) and TEA (3.4 g, 34 mmol, 1.0 eq). The resulting mixture was stirred for 4 h then concentrated. The residue was purified by silica gel column chromatography, eluting with THF/n-hexane (1/3) to afford 2-ethyl-4-nitro-3-phenylbutanal (3.8 g, 51% yield) as a light yellow oil.

LCMS (ES, m/z): 220 [M−H]⁻

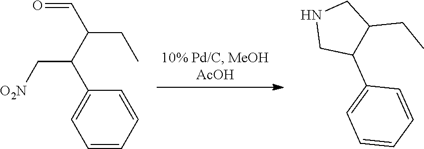 [SMILES: CCC(C=O)C(C[N+](=O)[O-])c1ccccc1 ; CCC1CNCC1c1ccccc1]

2-Ethyl-4-nitro-3-phenylbutanal (3.0 g, 14 mmol), acetic acid (0.8 g, 14 mmol) and 10% Pd/C (1.5 g) in MeOH (30 mL) were stirred for 16 h under hydrogen atmosphere. The resulting mixture was filtered, and the filter cake washed with MeOH (3×30 mL). The combined filtrate was concentrated and the residue dissolved in EtOAc (5 mL). The mixture was acidified to pH 4 with 2 M HCl in EtOAc, and the precipitated solids were collected by filtration and washed with petroleum ether (3×10 mL) to afford 3-ethyl-4-phenylpyrrolidine hydrochloride (2 g, 84% yield).

LCMS (ES, m/z): 176 [M+H]⁺.

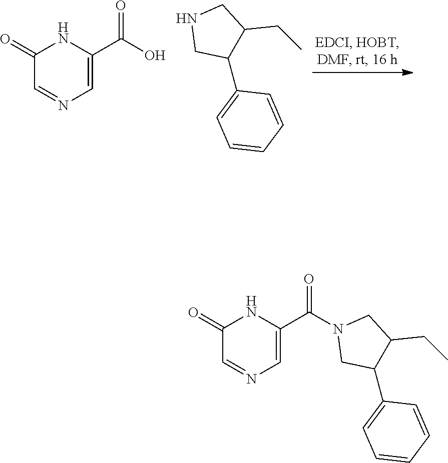

To 6-oxo-1H-pyrazine-2-carboxylic acid (0.53 g, 3.8 mmol) in DMF (10 mL) was added HOBT (1.3 g, 9.4 mmol, 2.5 eq) and EDCl (0.87 g, 4.5 mmol, 1.2 eq). 3-Ethyl-4-phenylpyrrolidine hydrochloride (1.6 g, 7.6 mmol, 2 eq) was added and the mixture stirred for 2 h. The resulting mixture was filtered, and the filter cake washed with MeOH (2×3 mL). The combined filtrate was concentrated and the residue purified by Prep-HPLC (Column: Welch Xtimate C18 ExRS, 250 mm, 10 μm; Mobile Phase: 15-60% MeCN/0.05% aqueous ammonia over 10 min.; Flow rate: 90 mL/min; Wavelength: 254 nm) to afford 6-(3-ethyl-4-phenylpyrrolidine-1-carbonyl)-1H-pyrazin-2-one (350 mg, 31% yield) as an off-white solid.

LCMS (ES, m/z): 298 [M+H]⁺

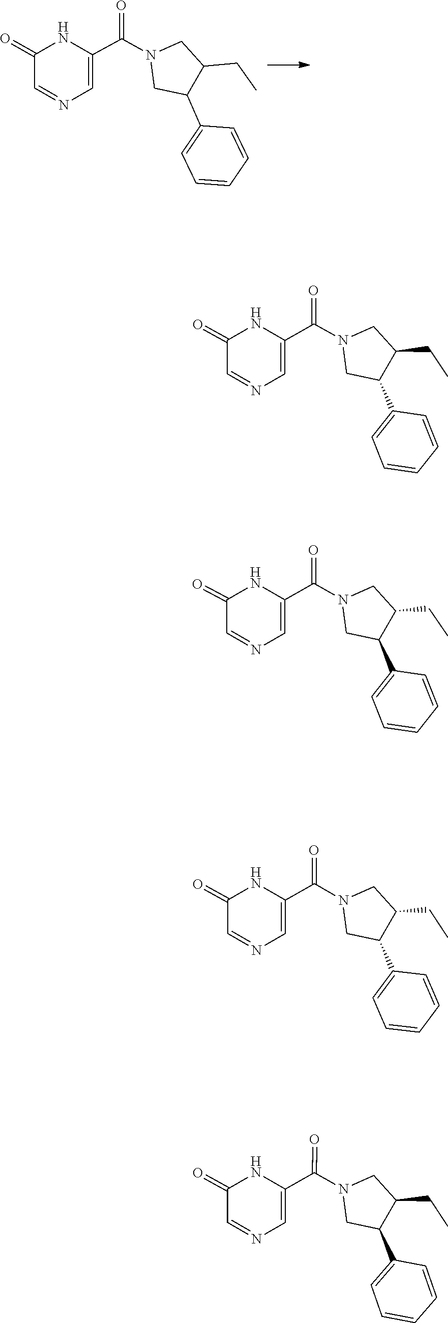 [SMILES: CCC1CN(C(=O)c2cncc(=O)[nH]2)CC1c1ccccc1 ; CC[C@@H]1CN(C(=O)c2cncc(=O)[nH]2)C[C@H]1c1ccccc1 ; CC[C@H]1CN(C(=O)c2cncc(=O)[nH]2)C[C@@H]1c1ccccc1 ; CC[C@H]1CN(C(=O)c2cncc(=O)[nH]2)C[C@H]1c1ccccc1 ; CC[C@@H]1CN(C(=O)c2cncc(=O)[nH]2)C[C@@H]1c1ccccc1]

6-(3-Ethyl-4-phenylpyrrolidine-1-carbonyl)-1H-pyrazin-2-one (250 mg, 0.8 mmol) was separated by Prep-HPLC (Column: CHIRALPAK AD-H, 2×25 cm, 5 μm; Mobile Phase: 20% EtOH/Hexane (containing 0.1% TFA); Flow rate: 25 mL/min; Wavelength: 220 nm) to give:

Ex. 15(a) Diastereomer A (Rt 5.0 min.) was isolated as a yellow solid (110 mg).

LCMS (ES, m/z): 298 [M+H]⁺, method LCMS2.

1H NMR (400 MHz, DMSO-d6, ppm) δ 11.82 (br, s, 1H), 8.32-8.08 (m, 2H), 7.43-7.16 (m, 5H), 4.11-3.84 (m, 2H), 3.77-3.12 (m, 2H), 3.05-2.98 (m, 1H), 2.40-2.20 (m, 1H), 1.50-1.11 (m, 2H), 0.94-0.62 (m, 3H).

Ex. 15(b) Diastereomer B (Rt 5.6 min.) was isolated as a yellow solid (97 mg).

LCMS (ES, m/z): 298 [M+H]⁺, method LCMS2.

1H NMR (400 MHz, DMSO-d6, ppm) δ 11.82 (br, s, 1H), 8.36-8.07 (m, 2H), 7.42-7.19 (m, 5H), 4.12-3.89 (m, 2H), 3.75-3.10 (m, 2H), 3.06-2.98 (m, 1H), 2.36-2.18 (m, 1H), 1.44-1.15 (m, 2H), 0.93-0.65 (m, 3H).

Ex. 15(c) Diastereomer C (Rt 11.5 min.) was isolated as a light brown solid (3.9 mg).

LCMS (ES, m/z): 298 [M+H]⁺, method LCMS2.

1H NMR (400 MHz, DMSO-d6, ppm) δ 11.82 (br, s, 1H), 8.48-8.10 (m, 2H), 7.40-7.31 (m, 2H), 7.28-7.17 (m, 2H), 7.13 (d, J=7.6 Hz, 1H), 4.10-3.66 (m, 3H), 3.57-3.52 (m, 1H), 3.47-3.27 (m, 1H), 2.44-2.36 (m, 1H), 1.06 (t, J=7.0 Hz, 2H), 0.88-0.68 (m, 3H).

Ex. 15(d) Diastereomer D (R^{t} 10.0 min.) was isolated as a light brown solid (4.7 mg).

LCMS (ES, m/z): 298 [M+H]⁺, method LCMS2.

1H NMR (400 MHz, DMSO-d6, ppm) δ 8.25 (s, 1H), 8.19 (d, J=10.5 Hz, 1H), 7.40-7.30 (m, 2H), 7.28-7.18 (m, 2H), 7.13 (d, J=7.6 Hz, 1H), 4.12-3.69 (m, 3H), 3.56-3.49 (m, 1H), 3.47-3.25 (m, 1H), 2.46-2.36 (m, 1H), 1.08-0.86 (m, 2H), 0.85-0.75 (m, 3H).

The Diastereomers A to D are (stereochemistry not formally allocated):
(R),(R)-6-(3-ethyl-4-phenylpyrrolidine-1-carbonyl)pyrazin-2(1H)-one,
(S),(S)-6-(3-ethyl-4-phenylpyrrolidine-1-carbonyl)pyrazin-2(1H)-one,
(R),(S)-6-(3-ethyl-4-phenylpyrrolidine-1-carbonyl)pyrazin-2(1H)-one, and
(S),(R)-6-(3-ethyl-4-phenylpyrrolidine-1-carbonyl)pyrazin-2(1H)-one.

### Example 16(a) and Example 16(b): 6-[8-(4-Chlorophenyl)-6-azaspiro[3.4]octane-6-carbonyl]-1H-pyrazin-2-one Enantiomer A and 6-[8-(4-Chlorophenyl)-6-azaspiro[3.4]octane-6-carbonyl]-1H-pyrazin-2-one Enantiomer B

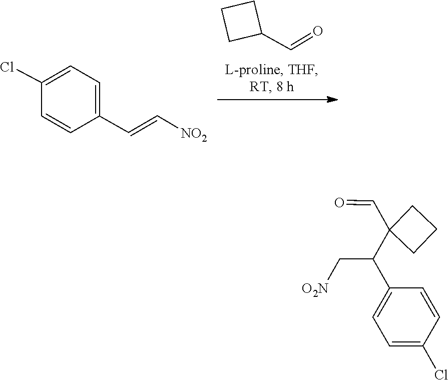 [SMILES: O=CC1CCC1 ; O=[N+]([O-])/C=C/c1ccc(Cl)cc1 ; O=CC1(C(C[N+](=O)[O-])c2ccc(Cl)cc2)CCC1]

To a stirred mixture of 4-chloro beta-nitrostyrene (5.0 g, 27 mmol) and cyclobutanecarbaldehyde (6.9 g, 82 mmol) in THF (50 mL) was added TEA (5.5 g, 54 mmol) and L-proline (0.94 g, 8.2 mmol). The resulting mixture was stirred for 8 h and concentrated. The residue was purified by silica gel column chromatography, eluting with n-hexane/EA (5/1) to afford 1-[1-(4-chlorophenyl)-2-nitroethyl]cyclobutane-1-carbaldehyde (3 g, 41% yield) as a light yellow oil.

LCMS (ES, m/z): 266 [M−H]⁻

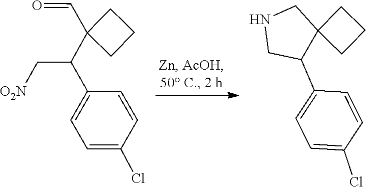 [SMILES: O=CC1(C(C[N+](=O)[O-])c2ccc(Cl)cc2)CCC1 ; Clc1ccc(C2CNCC23CCC3)cc1]

To 1-(1-(4-chlorophenyl)-2-nitroethyl)cyclobutane-1-carbaldehyde (0.93 g, 3.5 mmol) in EtOH (18 mL), was added zinc powder (1.1 g, 18 mmol, 5 eq) and AcOH (1.7 g, 28 mmol, 8 eq). The reaction mixture was stirred for 2 h at 60° C., then cooled and filtered, and the filter cake was washed with EtOAc (3×20 mL). The combined filtrate was concentrated, and the residue taken up in 2M HCl in EtOAc (30 mL). The resulting mixture was stirred for 2 h then concentrated. The residue was triturated with EtOAc and Petroleum ether to afford 8-(4-chlorophenyl)-6-azaspiro[3.4]octane hydrochloride (500 mg, 64% yield) as a white solid.

LCMS (ES, m/z): 222 [M+H]⁺

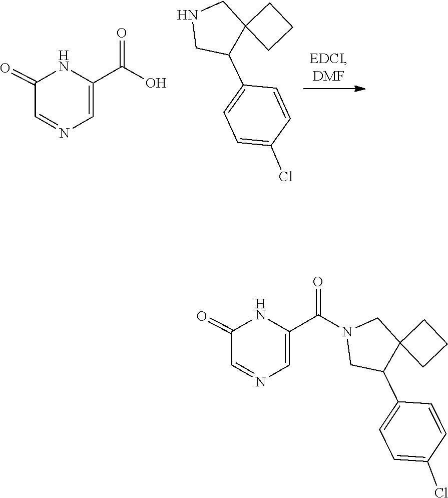 [SMILES: O=C(O)c1cncc(=O)[nH]1 ; Clc1ccc(C2CNCC23CCC3)cc1 ; O=C(c1cncc(=O)[nH]1)N1CC(c2ccc(Cl)cc2)C2(CCC2)C1]

To a stirred solution of 8-(4-chlorophenyl)-6-azaspiro[3.4]octane (100 mg, 0.4 mmol) and 6-oxo-1H-pyrazine-2-carboxylic acid (76 mg, 0.5 mmol, 1.2 eq) in DMF(1 mL) was added EDC.HCl (140 mg, 0.9 mmol, 2 eq) in portions. The reaction mixture was stirred for 6 h, then quenched with water. The mixture was purified by Prep-HPLC (Column: Welch Xtimate C18 ExRS, 250 mm, 10 μm; Mobile Phase: 20-75% MeCN/0.05% aqueous ammonia over 10 min.; Flow rate: 80 mL/min) to afford 6-[8-(4-chlorophenyl)-6-azaspiro[3.4]octane-6-carbonyl]-1H-pyrazin-2-one (80 mg, 52% yield) as a white solid.

LCMS (ES, m/z): 344 [M+H]⁺

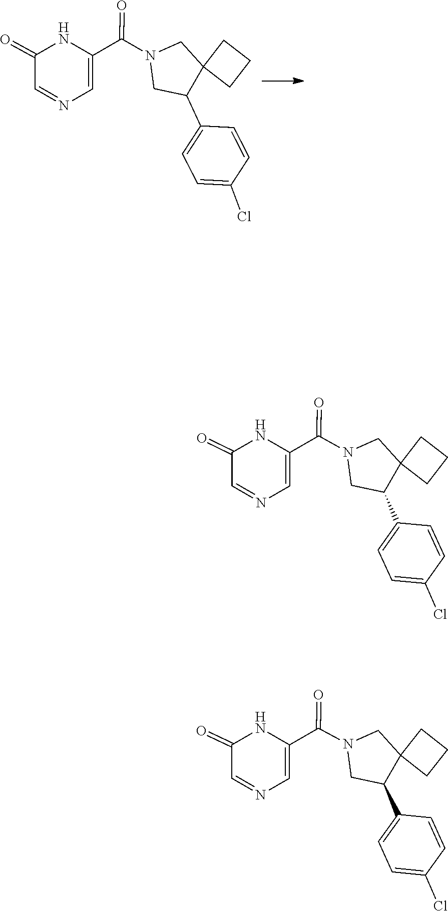 [SMILES: O=C(c1cncc(=O)[nH]1)N1CC(c2ccc(Cl)cc2)C2(CCC2)C1 ; O=C(c1cncc(=O)[nH]1)N1C[C@@H](c2ccc(Cl)cc2)C2(CCC2)C1 ; O=C(c1cncc(=O)[nH]1)N1C[C@H](c2ccc(Cl)cc2)C2(CCC2)C1]

6-[8-(4-Chlorophenyl)-6-azaspiro[3.4]octane-6-carbonyl]-1H-pyrazin-2-one (70 mg) was separated by SFC Column: CHIRALPAK IH, 3×25 cm, 5 μm; Mobile Phase: 30% [2:1 MeOH:DCM containing 0.1% 2M ammonia in MeOH]/CO₂; Flow rate: 80 mL/min; Wavelength: 220 nm to give:

Ex. 16(a) Enantiomer A (Rt 4.4 min.) was isolated as a yellow solid (16 mg, 23% yield).

LCMS (ES, m/z): 344 [M+H]⁺, method LCMS2.

1H NMR (400 MHz, DMSO-d6, ppm) δ 8.26-8.02 (m, 2H), 7.42 (t, J=8.0 Hz, 2H), 7.33-7.24 (m, 2H), 4.08-3.79 (m, 2H), 3.78-7.73 (m, 2H), 3.66 (s, 1H), 2.03-1.85 (m, 2H), 1.82-1.43 (m, 4H).

Ex. 16(b) Enantiomer B (R^{t} 5.6 min.) was isolated as a yellow solid (14 mg, 19% yield).

LCMS (ES, m/z): 344 [M+H]⁺, method LCMS2.

¹H NMR (400 MHz, DMSO-d₆, ppm) δ 8.24-8.05 (m, 2H), 7.42 (t, J=8.1 Hz, 2H), 7.33-7.24 (m, 2H), 4.05-3.79 (m, 2H), 3.79-3.72 (m, 2H), 3.65 (s, 1H), 2.07-1.85 (m, 2H), 1.84-1.43 (m, 4H).

One of Enantiomers A and B is (S)-6-[8-(4-Chlorophenyl)-6-azaspiro[3.4]octane-6-carbonyl]-1H-pyrazin-2-one and the other is (R)-6-[8-(4-Chlorophenyl)-6-azaspiro[3.4]octane-6-carbonyl]-1H-pyrazin-2-one.

### Example 17(a) and Example 17(b): 6-[4-Phenyl-2-azaspiro[4.4]nonane-2-carbonyl]-1H-pyrazin-2-one Enantiomer A and 6-[4-phenyl-2-azaspiro[4.4]nonane-2-carbonyl]-1H-pyrazin-2-one Enantiomer B

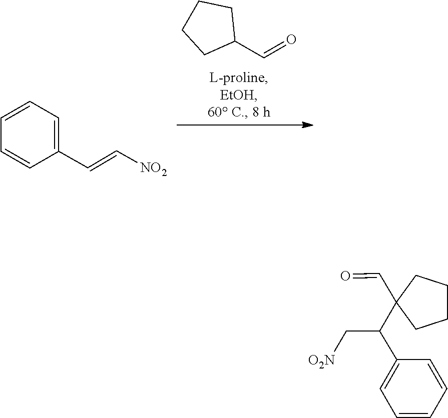 [SMILES: O=CC1CCCC1 ; O=[N+]([O-])/C=C/c1ccccc1 ; O=CC1(C(C[N+](=O)[O-])c2ccccc2)CCCC1]

A solution of (E)-(2-nitrovinyl)benzene (1.5 g, 10 mmol), L-proline (0.23 g, 2.0 mmol, 0.2 eq) and cyclopentanecarbaldehyde (3.0 g, 30 mmol, 3 eq) in EtOH (15 mL) was stirred overnight at 60° C. The reaction mixture was concentrated and the residue was purified by silica gel column chromatography, eluting with petroleum ether/EtOAc (5/1) to afford 1-[1-(4-bromophenyl)-2-nitroethyl]cyclobutane-1-carbaldehyde (2 g, 80% yield) as a light yellow oil.

LCMS (ES, m/z): 246 [M−H]⁻

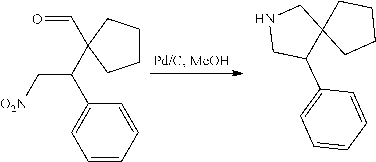 [SMILES: O=CC1(C(C[N+](=O)[O-])c2ccccc2)CCCC1 ; c1ccc(C2CNCC23CCCC3)cc1]

To a solution of 1-(2-nitro-1-phenylethyl)cyclopentane-1-carbaldehyde (0.90 g, 3.6 mmol) in MeOH (20 mL) was added 10% Pd/C (500 mg) under nitrogen atmosphere. The mixture was hydrogenated for 2 h under hydrogen atmosphere, then filtered through a Celite pad and the filtrate concentrated. The residue was dissolved in MeOH (10 mL) and AcOH (210 uL, 3.6 mmol, 1 eq) was added along with 10% Pd/C (400 mg). The mixture was stirred overnight under hydrogen atmosphere, then filtered through a Celite pad and concentrated under reduced pressure to afford 4-phenyl-2-azaspiro[4.4]nonane (0.50 g, 68% yield) as a colourless oil.

LCMS (ES, m/z): 202 [M+H]⁺

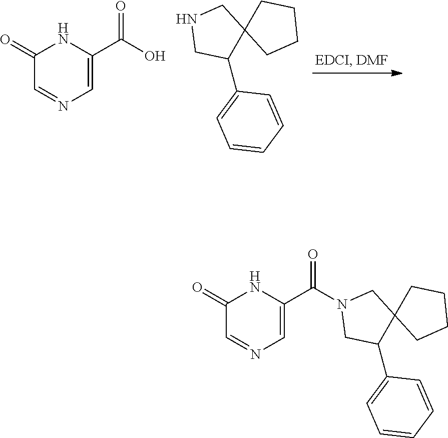

A solution of 4-phenyl-2-azaspiro[4.4]nonane (0.15 g, 0.7 mmol), EDCl (0.21 g, 1.1 mmol, 1.5 eq), HOBT (0.25 g, 1.8 mmol, 2.5 eq), DIPEA (0.27 g, 2.1 mmol, 3 eq) and 6-oxo-1H-pyrazine-2-carboxylic acid (0.12 g, 0.8 mmol, 1.2 eq) in DMF (2 mL) was stirred for 2 h then concentrated. The mixture was purified by Prep-HPLC (Column: XBridge C18, 19*150 mm, 5 μm; Mobile Phase: 10-65% MeCN/20 mM aqueous NH₄HCO₃ solution containing 0.05% NH₃·H₂O over 8 min.; Flow rate: 60 mL/min.) to afford 6-(4-phenyl-2-azaspiro[4.4]nonane-2-carbonyl)pyrazin-2(1H)-one (80 mg, 33% yield) as a white solid.

LCMS (ES, m/z): 324 [M+H]⁺

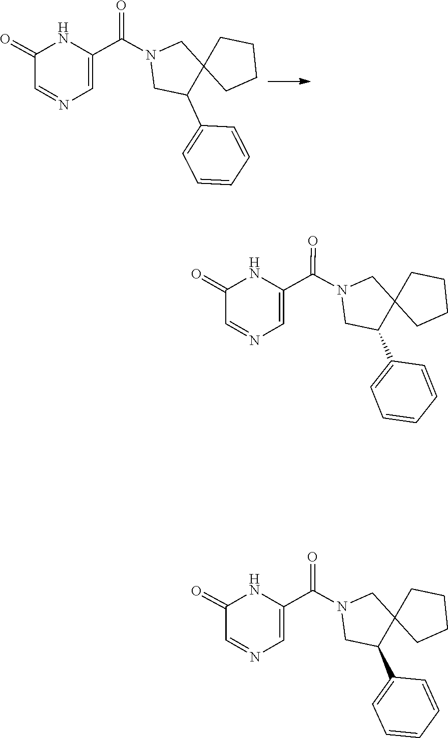 [SMILES: O=C(c1cncc(=O)[nH]1)N1CC(c2ccccc2)C2(CCCC2)C1 ; O=C(c1cncc(=O)[nH]1)N1C[C@@H](c2ccccc2)C2(CCCC2)C1 ; O=C(c1cncc(=O)[nH]1)N1C[C@H](c2ccccc2)C2(CCCC2)C1]

6-(4-Phenyl-2-azaspiro[4.4]nonane-2-carbonyl)pyrazin-2(1H)-one (80 mg) was separated by Prep-HPLC with the following conditions (Column: CHIRALPAKAY-3, 100*4.6 mm, 3 um, AY3000-AW003; Mobile Phase: 30% 1:1 EtOH/MeOH/n-Hexane) to give:

Ex. 17(a) Enantiomer A (first eluting isomer) as a white solid (24 mg, 30% yield).

LCMS (ES, m/z): 324 [M+H]⁺, method LCMS2.

¹H-NMR: (400 MHz, DMSO-d₆, ppm) δ 8.27-8.01 (m, 2H), 7.39-7.16 (m, 5H), 4.10-3.78 (m, 2H), 3.67-3.43 (m, 2H), 3.26-3.21 (m, 1H), 1.68-1.31 (m, 6H), 1.29-1.05 (m, 2H).

Ex. 17(b) Enantiomer B (second eluting isomer) as a white solid (24 mg, 30% yield).

LCMS (ES, m/z): 324 [M+H]⁺, method LCMS2.

¹H-NMR: (400 MHz, DMSO-d₆, ppm) δ 8.24-8.03 (m, 2H), 7.39-7.13 (m, 5H), 4.11-3.76 (m, 2H), 3.67-3.43 (m, 2H), 3.26-3.22 (m, 1H), 1.68-1.31 (m, 6H), 1.27-1.04 (m, 2H).

One of Enantiomers A and B is (S)-6-[4-Phenyl-2-azaspiro[4.4]nonane-2-carbonyl]-1H-pyrazin-2-one and the other is (R)-6-[4-Phenyl-2-azaspiro[4.4]nonane-2-carbonyl]-1H-pyrazin-2-one.

### Example 18: rac-2-{8-phenyl-6-azaspiro[3.4]octane-6-carbonyl}-3H-pyrimidin-4-one

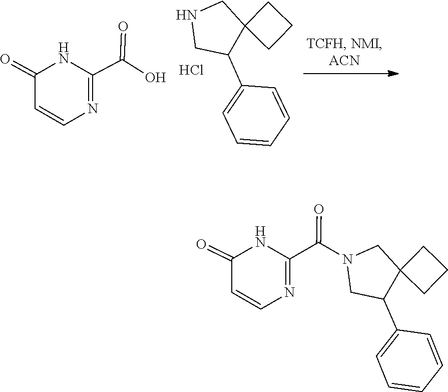 [SMILES: O=C(O)c1nccc(=O)[nH]1 ; Cl ; c1ccc(C2CNCC23CCC3)cc1 ; O=C(c1nccc(=O)[nH]1)N1CC(c2ccccc2)C2(CCC2)C1]

A solution of 4-oxo-3H-pyrimidine-2-carboxylic acid (0.42 g, 3.0 mmol), TCFH (1.2 g, 4.5 mmol), NMI (0.62 g, 7.5 mmol) in CH₃CN (3 mL) was stirred for 15 min. 8-Phenyl-6-azaspiro[3.4]octane hydrochloride (0.67 g, 3.6 mmol) was added in portions over 5 min. The resulting mixture was stirred for 1 h. The resulting mixture was quenched by water and extracted with CH₂Cl₂ (3×10 mL). The combined organic layers were washed with brine (15 mL), dried over anhydrous Na₂SO₄ and concentrated. The residue was purified by reverse-phase chromatography (Column: C18 silica gel; Mobile Phase: 10-50% MeCN in aqueous 0.1% NH₃ over 10 min; Detector: UV 254 nm) to afford racemic 2-{8-phenyl-6-azaspiro[3.4]octane-6-carbonyl}-3H-pyrimidin-4-one (12.5 mg, 1.4% yield) as a white solid.

LCMS (ES, m/z): 310 [M+H]⁺, method LCMS2.

¹H NMR: (400 MHz, DMSO-d₆, ppm) δ 12.74 (br, s, 1H), 8.11-7.85 (m, 1H), 7.42-7.33 (m, 2H), 7.32-7.21 (m, 3H), 6.46-6.34 (m, 1H), 4.06-3.86 (m, 1H), 3.86-3.71 (m, 2H), 3.70-3.59 (m, 1H), 3.31-3.25 (m, 1H), 2.07-1.91 (m, 2H), 1.89-1.60 (m, 2H), 1.60-1.47 (m, 2H).

### Example 19(a) and Example 19(b): 6-[(4-Ethynylphenyl)-6-azaspiro[3.4]octane-6-carbonyl]-1H-pyrazin-2-one Enantiomer A and 6-[(4-Ethynylphenyl)-6-azaspiro[3.4]octane-6-carbonyl]-1H-pyrazin-2-one Enantiomer B

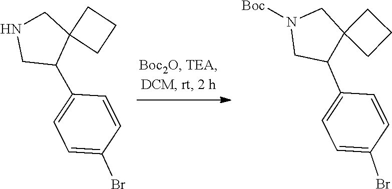 [SMILES: Brc1ccc(C2CNCC23CCC3)cc1 ; CC(C)(C)OC(=O)N1CC(c2ccc(Br)cc2)C2(CCC2)C1]

A solution of 8-(4-Bromophenyl)-6-azaspiro[3.4]octane (10 g, 38 mmol), TEA (11 g, 110 mmol) and Boc₂O (16 g, 75 mmol) in DCM (100 mL) was stirred for 2 h. The resulting mixture was concentrated and the residue purified by silica gel chromatography, eluting with n-hexane/EtOAc (5/1) to afford tert-butyl 8-(4-bromophenyl)-6-azaspiro[3.4]octane-6-carboxylate (8.0 g, 58% yield) as a reddish brown oil.

LCMS (ES, m/z): 366, 368 [M+H]⁺

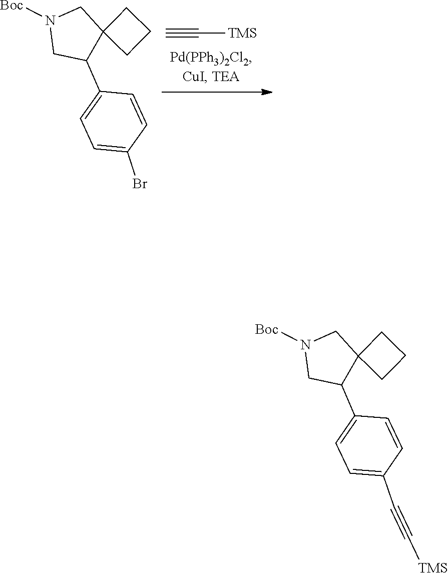

To a solution of tert-butyl 8-(4-bromophenyl)-6-azaspiro[3.4]octane-6-carboxylate (1.0 g, 2.7 mmol) in DMF (10 mL) was added trimethylsilylacetylene (1.1 g, 11 mmol), Pd(PPh₃)₂Cl₂ (0.19 g, 0.3 mmol), CuI (0.05 g, 0.27 mmol) and TEA (0.83 g, 8.2 mmol). The resulting mixture was stirred for 2 h at 75° C. The mixture was extracted with CH₂Cl₂ (3×10 mL). The combined organic layers were washed with brine (15 mL), dried over anhydrous Na₂SO₄ and concentrated. The residue was purified by silica gel column chromatography, eluting with n-hexane/EtOAc (5/1) to afford tert-butyl 8-{4-[2-(trimethylsilyl)ethynyl]phenyl}-6-azaspiro[3.4]octane-6-carboxylate (0.80 g, 76% yield) as a reddish brown oil.

LCMS (ES, m/z): 384 [M+H]⁺

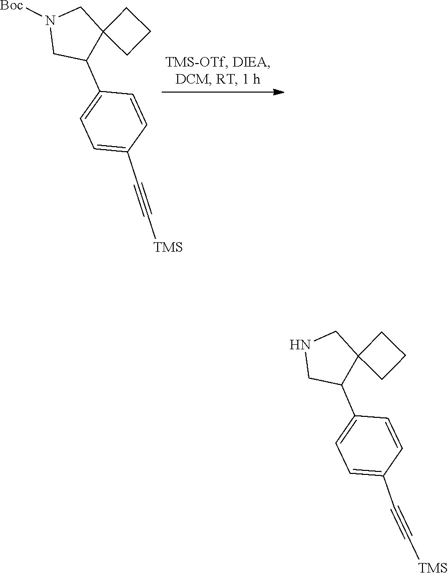 [SMILES: CC(C)(C)OC(=O)N1CC(c2ccc(C#C[Si](C)(C)C)cc2)C2(CCC2)C1 ; C[Si](C)(C)C#Cc1ccc(C2CNCC23CCC3)cc1]

To a solution of tert-butyl 8-{4-[2-(trimethylsilyl)ethynyl]phenyl}-6-azaspiro[3.4]octane-6-carboxylate (0.33 g, 0.9 mmol) in DCM (3.3 mL) was added DIPEA (0.33 g, 2.6 mmol) and TMSOTf (0.57 g, 2.6 mmol). The resulting mixture was stirred for 1 h then concentrated to afford 8-{4-[2-(trimethylsilyl)ethynyl]phenyl}-6-azaspiro[3.4]octane (0.76 g, crude) as a brown solid. The crude product was used in the next step without further purification.

LCMS (ES, m/z): 284 [M+H]⁺

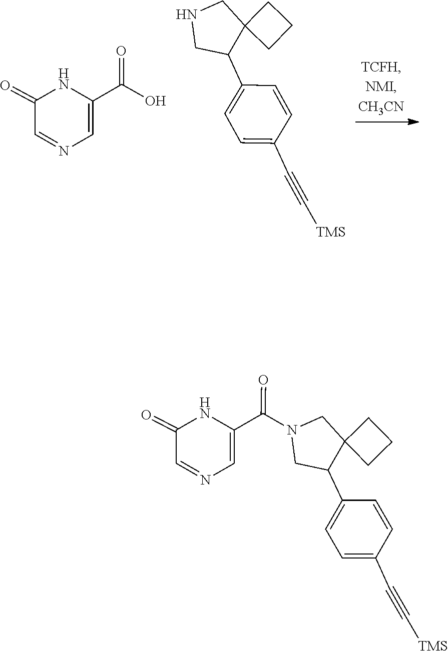 [SMILES: O=C(O)c1cncc(=O)[nH]1 ; C[Si](C)(C)C#Cc1ccc(C2CNCC23CCC3)cc1 ; C[Si](C)(C)C#Cc1ccc(C2CN(C(=O)c3cncc(=O)[nH]3)CC23CCC3)cc1]

A solution of 8-{4-[2-(trimethylsilyl)ethynyl]phenyl}-6-azaspiro[3.4]octane (0.76 g, 2.7 mmol), 6-oxo-1H-pyrazine-2-carboxylic acid (0.25 g, 1.8 mmol), TCFH (0.75 g, 2.7 mmol) and NMI (0.59 g, 7.1 mmol) in CH₃CN (2.5 mL) was stirred for 1 h then concentrated. The residue was purified by silica gel column chromatography, eluting with CH₂Cl₂/MeOH (10/1) to afford 6-(8-{4-[2-(trimethylsilyl)ethynyl]phenyl}-6-azaspiro[3.4]octane-6-carbonyl)-1H-pyrazin-2-one (0.46 g, 63% yield) as a dark red oil.

LCMS (ES, m/z): 406 [M+H]⁺

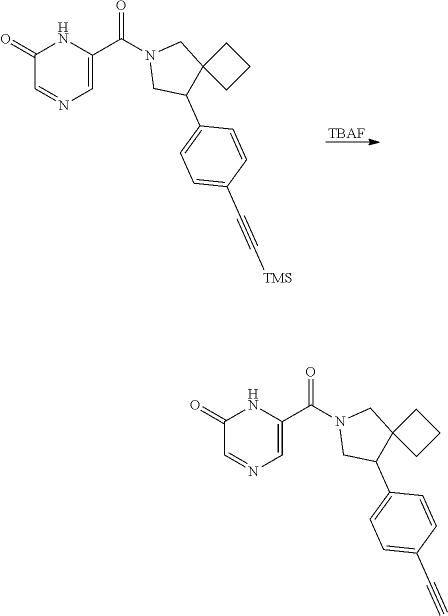 [SMILES: C[Si](C)(C)C#Cc1ccc(C2CN(C(=O)c3cncc(=O)[nH]3)CC23CCC3)cc1 ; "TBAF" ; C#Cc1ccc(C2CN(C(=O)c3cncc(=O)[nH]3)CC23CCC3)cc1]

To a solution of 6-(8-{4-[2-(trimethylsilyl)ethynyl]phenyl}-6-azaspiro[3.4]octane-6-carbonyl)-1H-pyrazin-2-one (390 mg, 1.0 mmol) in THF (4 mL) was added 1M TBAF in THF (2.8 mL, 2.8 mmol). The resulting mixture was stirred for 1 h then concentrated. The residue was purified by silica gel column chromatography, eluting with CH₂Cl₂/MeOH (10/1) to afford 6-[8-(4-ethynylphenyl)-6-azaspiro[3.4]octane-6-carbonyl]-1H-pyrazin-2-one (67 mg, 21% yield) as an off-white solid.

LCMS (ES, m/z): 334 [M+H]⁺

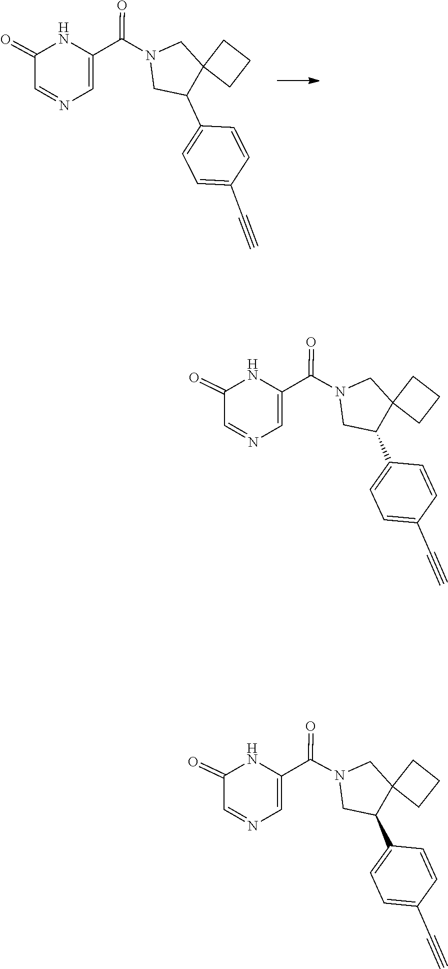 [SMILES: C#Cc1ccc(C2CN(C(=O)c3cncc(=O)[nH]3)CC23CCC3)cc1 ; C#Cc1ccc([C@@H]2CN(C(=O)c3cncc(=O)[nH]3)CC23CCC3)cc1 ; C#Cc1ccc([C@H]2CN(C(=O)c3cncc(=O)[nH]3)CC23CCC3)cc1]

6-[8-(4-Ethynylphenyl)-6-azaspiro[3.4]octane-6-carbonyl]-1H-pyrazin-2-one (67 mg, 0.2 mmol) was separated by Prep-HPLC with the following conditions (Column: XA CHIRALPAK IA, 2*25 cm, 5 μm; Mobile Phase: 50% 1:1 EtOH:DCM/Hexane (containing 0.2% FA); Flow rate: 25 mL/min; Wavelength: 254 nm) to afford:

Ex. 19(a) Enantiomer A (R^{t}=3.2 min.) as an off-white solid (15 mg, 23% yield)

LCMS (ES, m/z): 334 [M+H]⁺ 334, method LCMS2

¹H NMR: (400 MHz, DMSO-d₆, ppm) δ 11.91 (br, s, 1H), 8.31 (br, s, 1H), 8.18 (d, J=11.4 Hz, 1H), 7.48 (t, J=8.1 Hz, 2H), 7.31 (d, J=8.0 Hz, 1H), 7.25 (d, J=8.1 Hz, 1H), 4.17 (s, 1H), 4.03-3.82 (m, 1H), 3.82-3.70 (m, 1H), 3.69-3.68 (m, 1H), 3.67 (s, 1H), 3.31 (s, 1H), 2.06-1.88 (m, 2H), 1.87-1.62 (m, 2H), 1.61-1.46 (m, 2H).

Ex. 19(b) Enantiomer B (Rt=4.2 min.) as an off-white solid § (16 mg, 25% yield)

LCMS (ES, m/z): 334 [M+H]⁺, method LCMS2.

¹H NMR: (400 MHz, DMSO-d₆, ppm) δ 11.91 (br, s, 1H), 8.31 (br, s, 1H), 8.20-8.16 (m, 1H), 7.48 (t, J=8.0 Hz, 2H), 7.25-7.16 (m, 2H), 4.17 (s, 1H), 4.10-3.89 (m, 2H), 3.85-3.75 (m, 2H), 3.67 (s, 1H), 3.31 (s, 1H), 2.06-1.87 (m, 2H), 1.80-1.62 (m, 2H), 1.60-1.46 (m, 2H).

One of Enantiomers A and B is (S)-6-[(4-Ethynylphenyl)-6-azaspiro[3.4]octane-6-carbonyl]-1H-pyrazin-2-one and the other is (R)-6-[(4-Ethynylphenyl)-6-azaspiro[3.4]octane-6-carbonyl]-1H-pyrazin-2-one.

### Example 20(a) and Example 20(b): 4-[6-(6-oxo-1H-pyrazine-2-carbonyl)-6-azaspiro[3.4]octan-8-yl]benzonitrile Enantiomer A and 4-[6-(6-oxo-1H-pyrazine-2-carbonyl)-6-azaspiro[3.4]octan-8-yl]benzonitrile Enantiomer B

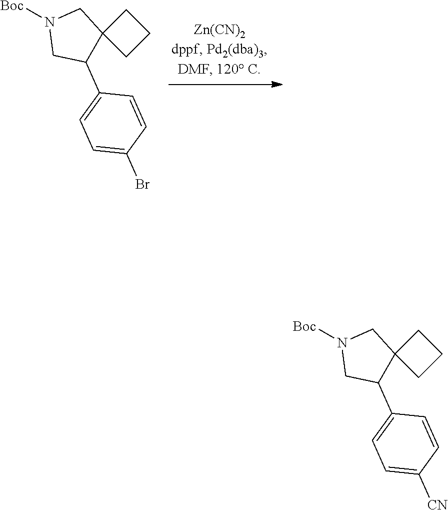 [SMILES: CC(C)(C)OC(=O)N1CC(c2ccc(Br)cc2)C2(CCC2)C1 ; CC(C)(C)OC(=O)N1CC(c2ccc(C#N)cc2)C2(CCC2)C1]

A mixture of tert-butyl 8-(4-bromophenyl)-6-azaspiro[3.4]octane-6-carboxylate (1.0 g, 2.7 mmol), zinc cyanide (1.6 g, 14 mmol), dppf (1.2 g, 2.2 mmol) and Pd₂(dba)₃ (0.25 g, 0.3 mmol) in DMF (20 mL) was stirred for 2 hours at 120° C. then cooled and diluted with water (30 mL). The mixture was extracted with EtOAc (3×50 mL). The combined organic layers were washed with brine (50 mL), dried over anhydrous Na₂SO₄ and concentrated. The residue was purified by silica gel column chromatography, eluting with petroleum ether/EtOAc (2/1) to afford tert-butyl 8-(4-cyanophenyl)-6-azaspiro[3.4]octane-6-carboxylate (0.43 g, 50% yield) as a white solid.

LCMS (ES, m/z): 313 [M+H]⁺

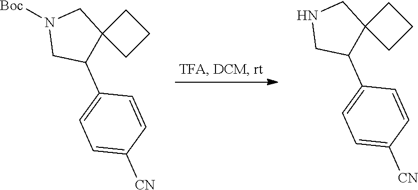 [SMILES: CC(C)(C)OC(=O)N1CC(c2ccc(C#N)cc2)C2(CCC2)C1 ; N#Cc1ccc(C2CNCC23CCC3)cc1]

A solution of tert-butyl 8-(4-cyanophenyl)-6-azaspiro[3.4]octane-6-carboxylate (0.30 g, 1.0 mmol) and TFA (1 mL) in DCM (3 mL) was stirred for 2 h. The mixture was neutralized to pH 7 with NaHCO₃ solution and extracted with EtOAc (3×5 mL). The combined organic layers were washed with brine (8 mL), dried over anhydrous Na₂SO₄ and concentrated. The residue was purified by silica gel column chromatography, eluting with petroleum ether/EtOAc (1/1) to afford 4-(6-azaspiro[3.4]octan-8-ylbenzonitrile (200 mg, 98% yield) as a white solid.

LCMS (ES, m/z): 213 [M+H]⁺

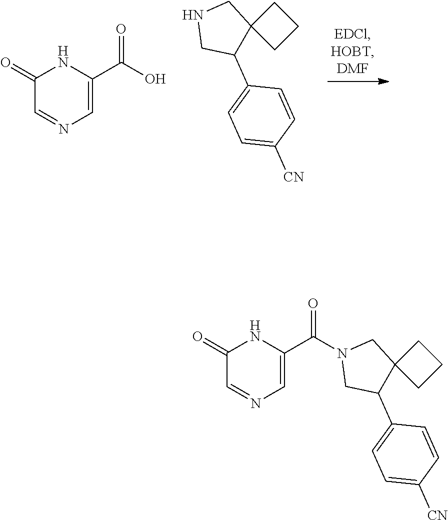 [SMILES: O=C(O)c1cncc(=O)[nH]1 ; N#Cc1ccc(C2CNCC23CCC3)cc1 ; N#Cc1ccc(C2CN(C(=O)c3cncc(=O)[nH]3)CC23CCC3)cc1]

A mixture of 4-(6-azaspiro[3.4]octan-8-ylbenzonitrile (0.19 g, 0.9 mmol), 6-oxo-1H-pyrazine-2-carboxylic acid (0.15 g, 1.1 mmol), EDCl (0.34 g, 1.8 mmol) and HOBT (0.30 g, 2.2 mmol) in DMF (4 mL) was stirred for 2 h. The resulting mixture was diluted with water (5 mL) and extracted with EtOAc (3×10 mL). The combined organic layers were washed with brine (15 mL), dried over anhydrous Na₂SO₄ and concentrated. The residue was purified by silica gel column chromatography, eluting with Petroleum ether/EtOAc (1/1) to afford 4-[6-(6-oxo-1H-pyrazine-2-carbonyl)-6-azaspiro[3.4]octan-8-yl]benzonitrile (80 mg, 26% yield) as a white solid.

LCMS (ES, m/z): 335 [M+H]⁺

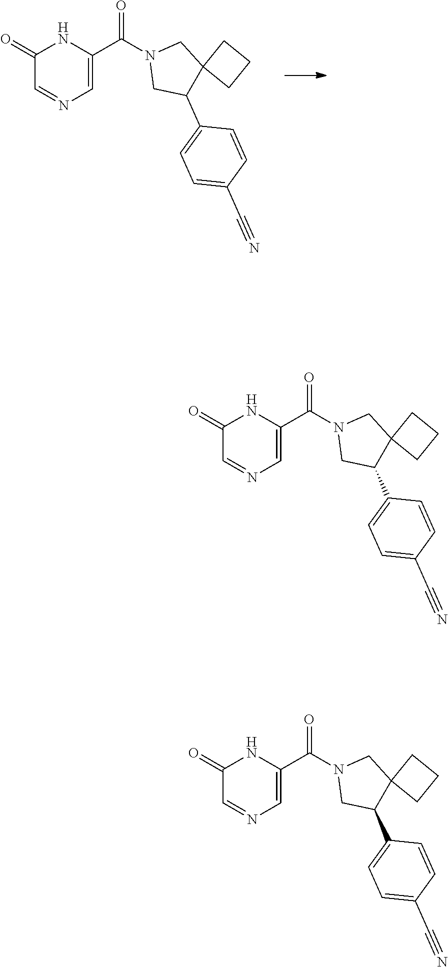 [SMILES: N#Cc1ccc(C2CN(C(=O)c3cncc(=O)[nH]3)CC23CCC3)cc1 ; N#Cc1ccc([C@@H]2CN(C(=O)c3cncc(=O)[nH]3)CC23CCC3)cc1 ; N#Cc1ccc([C@H]2CN(C(=O)c3cncc(=O)[nH]3)CC23CCC3)cc1]

4-[6-(6-oxo-1H-pyrazine-2-carbonyl)-6-azaspiro[3.4]octan-8-yl]benzonitrile (40 mg, 0.1 mmol) was separated by Prep-SFC (Column: CHIRALPAK ID, 3×25 cm, 5 μm; Mobile Phase: 45% 2:1 MeOH:DCM/CO₂; Flow rate: 80 mL/min; Wavelength: 220 nm) to afford: Ex. 20(a) Enantiomer A (Rt=7.8 min.) as an off-white solid(13 mg, 34% yield)

LCMS (ES, m/z): 335 [M+H]⁺, method LCMS2

¹H NMR (300 MHz, DMSO-d₆, ppm) δ 12.05 (br, s, 1H), 8.27 (br, s, 1H), 8.18 (d, J=8.6 Hz, 1H), 7.84 (dd, J=8.0, 5.2 Hz, 2H), 7.51 (d, J=8.1 Hz, 1H), 7.45 (d, J=8.0 Hz, 1H), 4.12-3.57 (m, 4H), 3.47-340 (m, 1H), 2.06-1.35 (m, 6H).

Ex. 20(b) Enantiomer B (R^{t}=3.8 min.) as an off-white solid, (22 mg, 55% yield)

LCMS (ES, m/z): 335 [M+H]⁺, method LCMS2.

¹H NMR (300 MHz, DMSO-d₆, ppm) δ 12.05 (br, s, 1H), 8.27 (br, s, 1H), 8.18 (d, J=8.6 Hz, 1H), 7.84 (dd, J=8.0, 5.2 Hz, 2H), 7.51 (d, J=8.1 Hz, 1H), 7.45 (d, J=8.0 Hz, 1H), 4.12-3.57 (m, 4H), 3.47-3.40 (m, 1H), 2.06-1.35 (m, 6H).

One of Enantiomers A and B is (S) 4-[6-(6-oxo-1H-pyrazine-2-carbonyl)-6-azaspiro[3.4]octan-8-yl]benzonitrile and the other is (R)-4-[6-(6-oxo-1H-pyrazine-2-carbonyl)-6-azaspiro[3.4]octan-8-yl]benzonitrile.

### Example 21(a) and Example 21(b): 2-[8-phenyl-6-azaspiro[3.4]octane-6-carbonyl]-3H-pyrimidin-4-one Enantiomer A and 2-[8-phenyl-6-azaspiro[3.4]octane-6-carbonyl]-3H-pyrimidin-4-one Enantiomer B

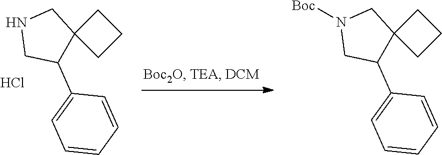 [SMILES: Cl ; c1ccc(C2CNCC23CCC3)cc1 ; CC(C)(C)OC(=O)N1CC(c2ccccc2)C2(CCC2)C1]

A solution of 8-phenyl-6-azaspiro[3.4]octane (0.80 g, 4.3 mmol), TEA (1.3 g, 13 mmol) and Boc₂O (1.1 g, 5.1 mmol) in DCM (8 mL) was stirred for 4 h. The mixture was concentrated and purified by silica gel column chromatography, eluting with CH₂Cl₂/MeOH (10/1) to afford tert-butyl 8-phenyl-6-azaspiro[3.4]octane-6-carboxylate (640 mg, 52% yield) as an off-white solid.

LCMS (ES, m/z): 288 [M+H]⁺

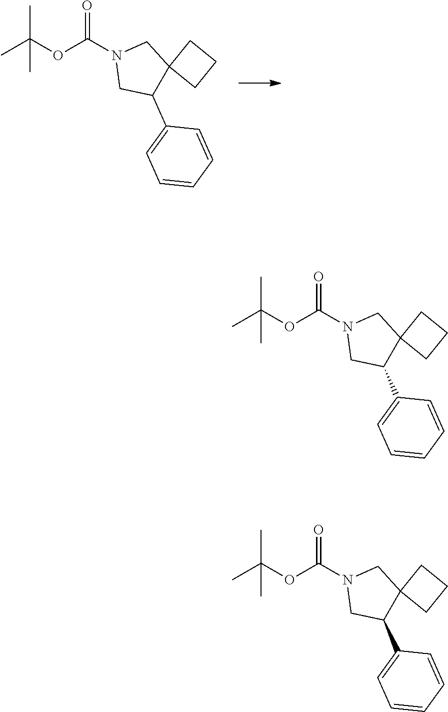 [SMILES: CC(C)(C)OC(=O)N1CC(c2ccccc2)C2(CCC2)C1 ; CC(C)(C)OC(=O)N1C[C@@H](c2ccccc2)C2(CCC2)C1 ; CC(C)(C)OC(=O)N1C[C@H](c2ccccc2)C2(CCC2)C1]

Tert-Butyl 8-phenyl-6-azaspiro[3.4]octane-6-carboxylate (390 mg, 1.4 mmol) was separated by Prep-HPLC with the following conditions (Column: XA-CHIRALPAK IG, 3*25 cm, 5 μm; Mobile Phase: 30% EtOH (containing 0.2% IPA)/3:1 Hexane:DCM; Flow rate: 35 mL/min;

Wavelength: 254 nm) to afford:

Enantiomer A (Rt=5.2 min.) as an reddish oil (130 mg, 33% yield)

LCMS (ES, m/z): 288 [M+H]⁺

Enantiomer B (Rt=7.0 min.) as an reddish oil (150 mg, 37% yield)

LCMS (ES, m/z): 288 [M+H]⁺

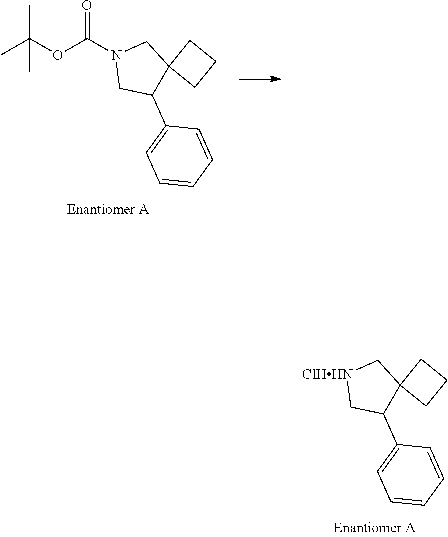

To a solution of tert-butyl 8-phenyl-6-azaspiro[3.4]octane-6-carboxylate (Enantiomer A) 145 mg, 0.5 mmol) in DCM (1.3 mL) was added 4M HCl in 1,4-dioxane (1.3 mL). The resulting mixture was stirred for 2 h and concentrated. The precipitated solids were collected by filtration and washed with EtOAc (1×2.0 mL) to afford 8-phenyl-6-azaspiro[3.4]octane hydrochloride Enantiomer A (90 mg, 95%) as an off-white solid.

LCMS (ES, m/z): 188 [M+H]⁺

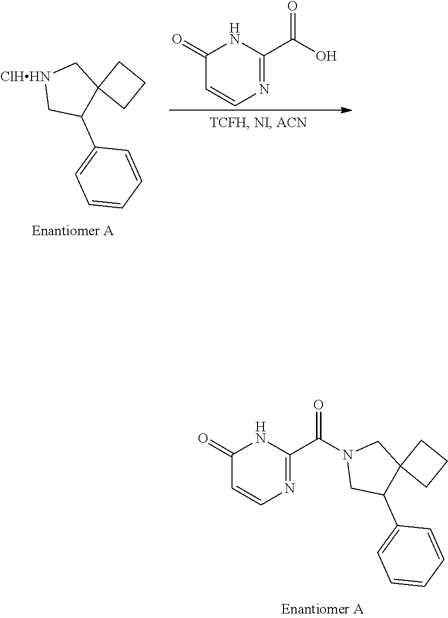

A solution of 4-oxo-3H-pyrimidine-2-carboxylic acid (70 mg, 0.5 mmol), TCFH (210 mg, 0.8 mmol) and NMI (100 mg, 1.3 mmol) in CH₃CN (0.7 mL) was stirred for 15 min. 8-Phenyl-6-azaspiro[3.4]octane hydrochloride enantiomer A (90 mg, 0.5 mmol) was added in portions over 5 min. The resulting mixture was stirred for 1 h then concentrated. The residue was purified by reversed-phase flash chromatography (Column, C18 silica gel; Mobile phase, 10-50% MeCN in 0.1% aqueous NH₃; Detector, UV 254 nm) to afford:

Ex. 21(a) Enantiomer A as an off-white solid (30 mg, 19% yield).

LCMS (ES, m/z): 310 [M+H]⁺, method LCMS02

¹H NMR: (400 MHz, DMSO-d₆, ppm) δ 8.00 (dd, J=17.9, 6.7 Hz, 1H), 7.40-7.33 (m, 2H), 7.32-7.19 (m, 3H), 6.42 (dd, J=12.1, 6.7 Hz, 1H), 4.06-3.87 (m, 1H), 3.85-3.70 (m, 2H), 3.69-3.59 (m, 1H), 3.30 (t, J=6.5 Hz, 1H), 2.07-1.90 (m, 2H), 1.90-1.70 (m, 2H), 1.67-1.49 (m, 2H).

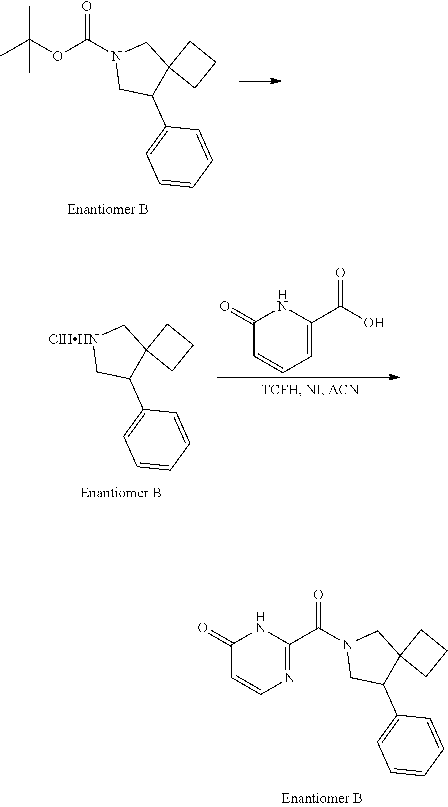

Enantiomer B was synthesised using an analogous method to afford:

Ex. 21(b) Enantiomer B as an off-white solid (35 mg, 21% yield).

LCMS (ES, m/z): 310 [M+H]⁺, method LCMS02

¹H NMR: (400 MHz, DMSO-d₆, ppm) δ 8.00 (dd, J=17.9, 6.7 Hz, 1H), 7.40-7.33 (m, 2H), 7.32-7.20 (m, 3H), 6.42 (dd, J=12.1, 6.7 Hz, 1H), 4.05-3.83 (m, 1H), 3.83-3.71 (m, 2H), 3.68-3.57 (m, 1H), 3.30 (t, J=6.5 Hz, 1H), 2.10-1.91 (m, 2H), 1.87-1.67 (m, 2H), 1.67-1.47 (m, 2H).

One of Enantiomers A and B is (S)-2-[8-phenyl-6-azaspiro[3.4]octane-6-carbonyl]-3H-pyrimidin-4-one and the other is (R)-2-[8-phenyl-6-azaspiro[3.4]octane-6-carbonyl]-3H-pyrimidin-4-one.

### Example 22: (3-hydroxyisoxazol-5-yl)(8-phenyl-6-azaspiro[3.4]octan-6-yl)methanone

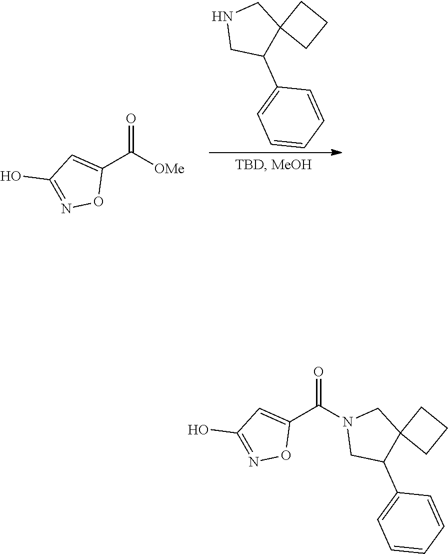 [SMILES: COC(=O)c1cc(O)no1 ; c1ccc(C2CNCC23CCC3)cc1 ; O=C(c1cc(O)no1)N1CC(c2ccccc2)C2(CCC2)C1]

To a solution of 8-phenyl-6-azaspiro[3.4]octane (0.2 g, 1.1 mmol) in MeOH (1 mL) was added methyl 3-oxo-2,3-dihydroisoxazole-5-carboxylate (0.2 g, 1.6 mmol) and TBD (0.4 g, 3.2 mmol). The solution was stirred for 4 h at 80° C. then concentrated. The residue was purified by Prep-HPLC (Column: YMC-Actus Triart C18, 30*150 mm, 5 μm; Mobile Phase: 30-60% MeCN/10 mmol/L aqueous NH₄HCO₃ containing 0.1% NH₃·H₂O over 7 min; Flow rate: 30 mL/min) to afford (3-hydroxyisoxazol-5-yl)(8-phenyl-6-azaspiro[3.4]octan-6-yl)methanone (180 mg, 54% yield) as a white solid.

LCMS (ES, m/z): 299 [M+H]⁺, method LCMS11

¹H NMR (300 MHz, DMSO-d₆, ppm) δ 11.80-11.62 (m, 1H), 7.44-7.34 (m, 2H), 7.31-7.18 (m, 3H), 6.61 (s, 1H), 4.12-3.92 (m, 1H), 3.86 (s, 1H), 3.84-3.74 (m, 1H), 3.74-3.58 (m, 1H), 3.31-3.27 (m, 1H), 2.06-1.93 (m, 2H), 1.92-1.66 (m, 2H), 1.66-1.51 (m, 1H), 1.50-1.34 (m, 1H).

### Examples 23(a) and Example 23(b): (3-hydroxyisoxazol-5-yl)(8-phenyl-6-azaspiro[3.4]octan-6-yl)methanone Enantiomer A and (3-hydroxyisoxazol-5-yl)(8-phenyl-6-azaspiro[3.4]octan-6-yl)methanone Enantiomer B

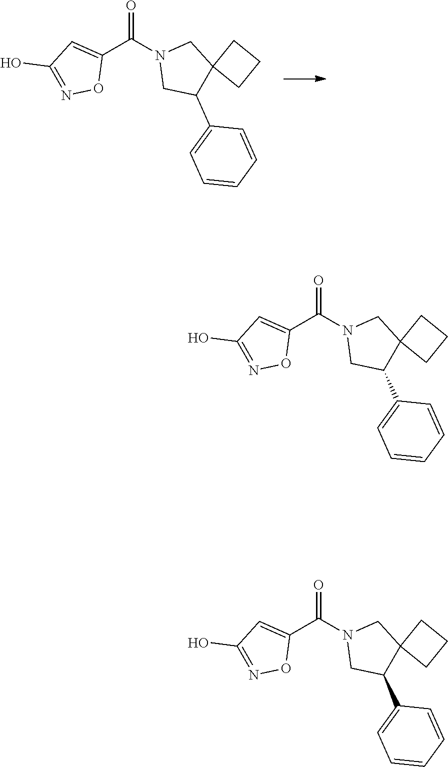 [SMILES: O=C(c1cc(O)no1)N1CC(c2ccccc2)C2(CCC2)C1 ; O=C(c1cc(O)no1)N1C[C@@H](c2ccccc2)C2(CCC2)C1 ; O=C(c1cc(O)no1)N1C[C@H](c2ccccc2)C2(CCC2)C1]

(3-hydroxyisoxazol-5-yl)(8-phenyl-6-azaspiro[3.4]octan-6-yl)methanone (70 mg, 0.23 mmol) was separated by Prep-HPLC (Column: CSH-C18, 19×250 mm, 5 μm; Mobile Phase: 10-80% MeCN/0.05% aqueous HCl; Flow rate: 20 mL/min; Wavelength: 254 nm/220 nm) to afford:

Ex. 23(a) Enantiomer A (first eluting isomer) as a white solid (22 mg, 31% yield).

LCMS (ES, m/z): 299 [M+H]⁺, method LCMS11

¹H NMR (400 MHz, DMSO-d₆, ppm) δ 11.87 (br, s, 1H), 7.38-7.32 (m, 2H), 7.29-7.21 (m, 3H), 6.56 (s, 1H), 4.09-3.95 (m, 1H), 3.87-3.73 (m, 2H), 3.73-3.58 (m, 1H), 3.27-3.23 (m, 1H), 2.08-1.95 (m, 2H), 1.87-1.67 (m, 2H), 1.66-1.53 (m, 1H), 1.53-1.39 (m, 1H).

Ex. 23(b) Enantiomer B (second eluting isomer) as a white solid (27 mg, 38% yield).

LCMS (ES, m/z): 299 [M+H]⁺, method LCMS11

¹H NMR (400 MHz, DMSO-d₆, ppm) δ 11.86 (br, s, 1H), 7.37-7.34 (m, 2H), 7.29-7.21 (m, 3H), 6.56 (s, 1H), 4.05-3.94 (m, 1H), 3.79-3.75 (m, 1H), 3.73-3.65 (m, 1H), 3.63-3.58 (m, 1H), 3.7-3.23 (m, 1H), 2.08-1.91 (m, 2H), 1.85-1.74 (m, 2H), 1.64-1.60 (m, 1H), 1.46-1.44 (m, 1H).

One of Enantiomers A and B is (S)-(3-hydroxyisoxazol-5-yl)(8-phenyl-6-azaspiro[3.4]octan-6-yl)methanone and the other is (R)-(3-hydroxyisoxazol-5-yl)(8-phenyl-6-azaspiro[3.4]octan-6-yl)methanone.

### Example 24: 3-[8-Phenyl-6-azaspiro[3.4]octane-6-carbonyl]-4H-1,2,4-oxadiazol-5-one

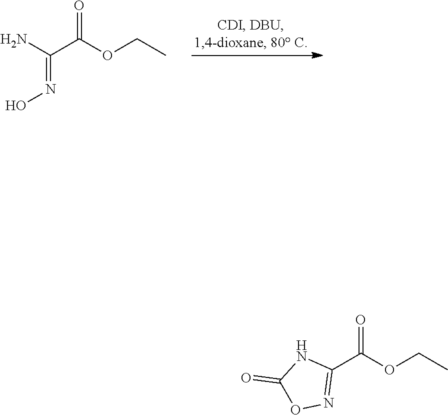

To a stirred solution of ethyl [(Z)—N′-hydroxycarbamimidoyl]formate (2.5 g, 19 mmol) in 1,4-dioxane (25 mL) at 80° C., CDI (3.7 g, 23 mmol) and DBU (3.5 g, 23 mmol) were added in portions. The resulting mixture was stirred for 2 h at 80° C. The reaction was quenched with 1M aqueous HCl at 0° C. The mixture was extracted with CH₂Cl₂ (3×100 mL). The combined organic layers were washed with brine (100 mL), dried over anhydrous Na₂SO₄ then concentrated. The residue was purified by silica gel column chromatography, eluting with CH₂Cl₂/MeOH (5/1) to afford ethyl 5-oxo-4H-1,2,4-oxadiazole-3-carboxylate (1.2 g, 40% yield) as a yellow solid.

LCMS (ES, m/z): 157 [M−H]⁻

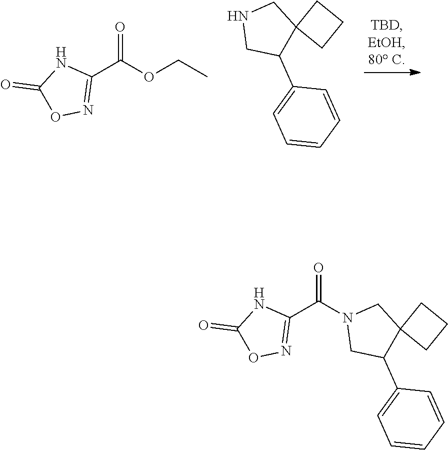 [SMILES: CCOC(=O)c1noc(=O)[nH]1 ; c1ccc(C2CNCC23CCC3)cc1 ; O=C(c1noc(=O)[nH]1)N1CC(c2ccccc2)C2(CCC2)C1]

5-Oxo-4H-1,2,4-oxadiazole-3-carboxylate (150 mg, 0.9 mmol), 8-phenyl-6-azaspiro[3.4]octane (270 mg, 1.4 mmol) and TBD (400 mg, 2.8 mmol) were stirred in EtOH (1.5 mL) at 80° C. for 2 h. The mixture was concentrated and the residue purified by reverse-phase flash chromatography (Column: C18 silica gel; Mobile phase: 10-50% MeCN in 0.1% aqueous FA; Wavelength: 254 nm) to afford 3-[8-phenyl-6-azaspiro[3.4]octane-6-carbonyl]-4H-1,2,4-oxadiazol-5-one (50 mg, 18% yield) as a white solid.

LCMS (ES, m/z): 300 [M+H]⁺, method LCMS12

¹H NMR: (400 MHz, DMSO-d₆, ppm) δ 13.07 (s, 1H), 7.41-7.33 (m, 2H), 7.33-7.22 (m, 3H), 4.15-3.99 (m, 1H), 3.96-3.87 (m, 1H), 3.86-3.73 (m, 1H), 3.70-3.62 (m, 1H), 3.31-3.24 (m, 1H), 2.03-1.91 (m, 2H), 1.88-1.71 (m, 2H), 1.69-1.43 (m, 2H).

### Examples 25 (a)-(d): 2-(2-fluoro-8-phenyl-6-azaspiro[3.4]octane-6-carbonyl)pyrimidin-4(3H)-one Diastereomer A, Diastereomer B, Diastereomer C and Diastereomer D

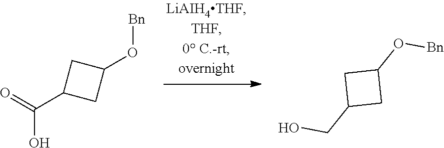 [SMILES: O=C(O)C1CC(OCc2ccccc2)C1 ; OCC1CC(OCc2ccccc2)C1]

To a solution of 3-(benzyloxy)cyclobutane-1-carboxylic acid (6.5 g, 32 mmol) in THF (65 mL) was added 2M LAH in THF (26 mL, 52 mmol) at 0° C. under and the resulting mixture was stirred overnight. The reaction was quenched by the addition of water/ice (25 mL) at 0° C. and 15% aqueous NaOH (7.2 ml). The mixture was extracted with EtOAc (3×100 mL), washed with brine (100 mL), dried over anhydrous Na₂SO₄ and concentrated. The residue was purified by silica gel column chromatography, eluting with n-hexane/EtOAc (3/1) to afford [3-(benzyloxy)cyclobutyl]methanol (4.5 g, 74% yield) as a colourless oil.

LCMS (ES, m/z): 193 [M+H]⁺

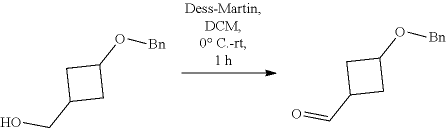 [SMILES: OCC1CC(OCc2ccccc2)C1 ; O=CC1CC(OCc2ccccc2)C1]

To a solution of [3-(benzyloxy)cyclobutyl]methanol (4.5 g, 23 mmol) in DCM (10 mL) at 0° C. was added Dess-Martin periodinane (12 g, 28 mmol) in portions. The resulting mixture was stirred for 1 h. The mixture was filtered, washed with Et₂O (2×10 mL) and concentrated. The residue was purified by silica gel column chromatography, eluting with n-hexane/EtOAc (2/1) to afford 3-(benzyloxy)cyclobutane-1-carbaldehyde (3.2 g, 71% yield) as a light yellow oil.

LCMS (ES, m/z): 191 [M+H]⁺

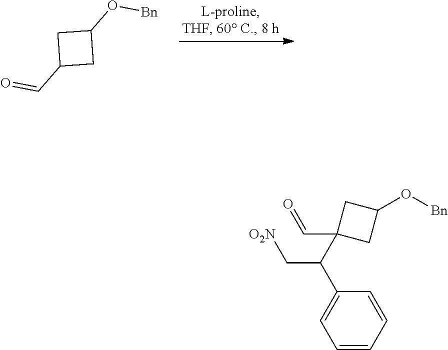 [SMILES: O=CC1CC(OCc2ccccc2)C1 ; O=CC1(C(C[N+](=O)[O-])c2ccccc2)CC(OCc2ccccc2)C1]

To a solution of p-nitrostyrene (860 mg, 5.7 mmol) and L-proline (0.20 g, 1.7 mmol) in THF (10 mL) was added 3-(benzyloxy)cyclobutane-1-carbaldehyde (3.3 g, 17 mmol) and Et₃N (0.58 g, 5.7 mmol). The resulting mixture was stirred for 8 h then concentrated. The residue was purified by silica gel column chromatography, eluting with n-hexane/EtOAc (2/1) to afford 3-(benzyloxy)-1-(2-nitro-1-phenylethyl)cyclobutane-1-carbaldehyde (1.7 g, 88% yield) as a white solid.

LCMS (ES, m/z): 338 [M−H]⁻

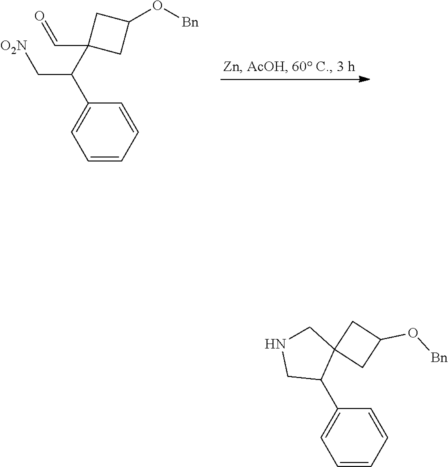 [SMILES: O=CC1(C(C[N+](=O)[O-])c2ccccc2)CC(OCc2ccccc2)C1 ; c1ccc(COC2CC3(CNCC3c3ccccc3)C2)cc1]

To a solution of 3-(benzyloxy)-1-(2-nitro-1-phenylethyl)cyclobutane-1-carbaldehyde (1.7 g, 5.0 mmol) in EtOH (16 mL) was added Zn (1.7 g, 25 mmol) and AcOH (2.5 g, 41 mmol). The resulting mixture was stirred for 3 h at 60° C. then filtered, washed with MeOH (3×6.0 mL) and concentrated. The residue was dissolved in 4M HCl in dioxane (10 mL) stirred for 10 min. then concentrated. The residue was triturated with hexane (15 mL) to afford 2-(benzyloxy)-8-phenyl-6-azaspiro[3.4]octane hydrochloride (1.2 g, 80% yield) as a white solid.

LCMS (ES, m/z): 294 [M+H]⁺

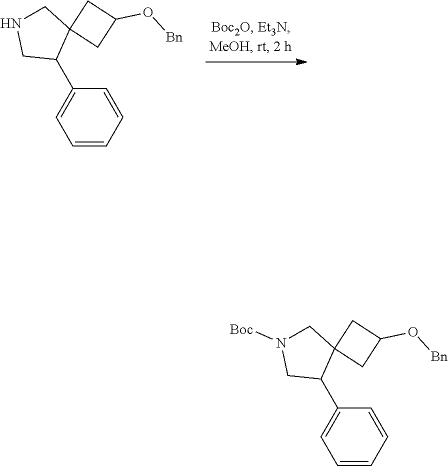 [SMILES: c1ccc(COC2CC3(CNCC3c3ccccc3)C2)cc1 ; CC(C)(C)OC(=O)N1CC(c2ccccc2)C2(CC(OCc3ccccc3)C2)C1]

2-(Benzyloxy)-8-phenyl-6-azaspiro[3.4]octane hydrochloride (1.2 g, 3.7 mmol), Et₃N (1.2 g, 11 mmol) and Boc₂O (890 mg, 4.4 mmol) were dissolved in MeOH (12 mL) and stirred for 3 h, before being concentrated. The residue was purified by silica gel column chromatography, eluting with n-hexane/EtOAc (2/1) to afford tert-butyl 2-(benzyloxy)-8-phenyl-6-azaspiro[3.4]octane-6-carboxylate (1.5 g, 93% yield) as a light yellow oil.

LCMS (ES, m/z): 394 [M+H]⁺

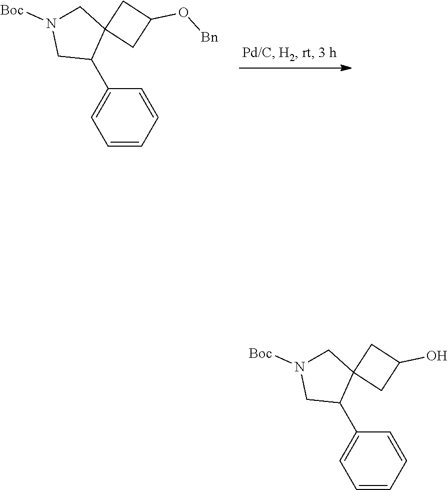 [SMILES: CC(C)(C)OC(=O)N1CC(c2ccccc2)C2(CC(OCc3ccccc3)C2)C1 ; CC(C)(C)OC(=O)N1CC(c2ccccc2)C2(CC(O)C2)C1]

To a solution of tert-butyl 2-(benzyloxy)-8-phenyl-6-azaspiro[3.4]octane-6-carboxylate (1.5 g, 3.8 mmol) in MeOH (20 mL) was added 10% Pd/C (1.2 g). The mixture was stirred for 3 h under hydrogen atmosphere, filtered through a Celite pad and concentrated. The residue was purified by silica gel column chromatography, eluting with n-hexane/EtOAc (1:1) to afford tert-butyl 2-hydroxy-8-phenyl-6-azaspiro[3.4]octane-6-carboxylate (700 mg, 60% yield) as a colourless oil.

LCMS (ES, m/z): 304 [M+H]⁺

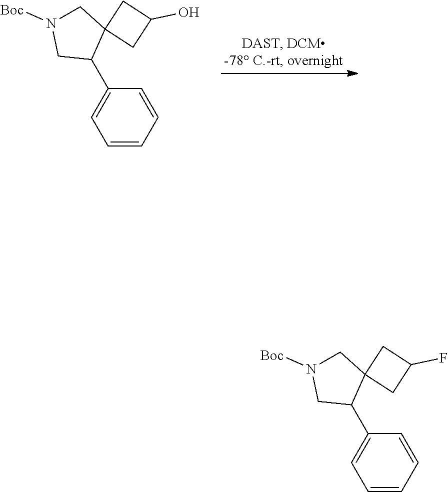 [SMILES: CC(C)(C)OC(=O)N1CC(c2ccccc2)C2(CC(O)C2)C1 ; CC(C)(C)OC(=O)N1CC(c2ccccc2)C2(CC(F)C2)C1]

A solution of tert-butyl 2-hydroxy-8-phenyl-6-azaspiro[3.4]octane-6-carboxylate (700 mg, 2.3 mmol) in DCM (10 mL) was cooled to −78° C. and DAST (740 mg, 4.6 mmol) was added dropwise. The resulting mixture was stirred overnight at room temperature then quenched with water/ice at 0° C. The mixture was extracted with DCM (3×10 mL), washed with brine (15 mL), dried over anhydrous Na₂SO₄ and concentrated afford tert-butyl 2-fluoro-8-phenyl-6-azaspiro[3.4]octane-6-carboxylate (340 mg, 48% yield) as a colourless oil.

LCMS (ES, m/z): 306 [M+H]⁺

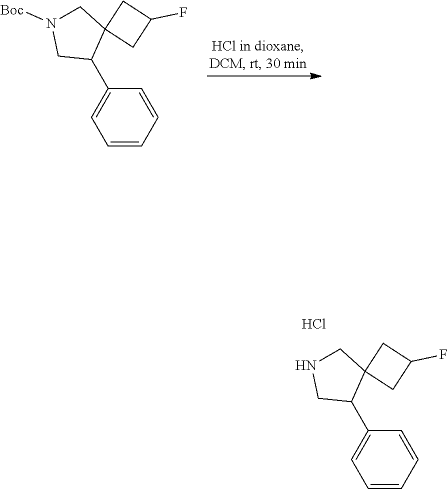 [SMILES: CC(C)(C)OC(=O)N1CC(c2ccccc2)C2(CC(F)C2)C1 ; Cl ; FC1CC2(CNCC2c2ccccc2)C1]

To a solution of tert-butyl 2-fluoro-8-phenyl-6-azaspiro[3.4]octane-6-carboxylate (340 mg, 1.1 mmol) in DCM (1.5 mL) was added 4M HCl in 1,4-dioxane (1.5 mL) dropwise. The resulting mixture was stirred for 30 min. then concentrated to afford 2-fluoro-8-phenyl-6-azaspiro[3.4]octane hydrochloride (145 mg, 53% yield) as a white solid.

LCMS (ES, m/z): 206 [M+H]⁺

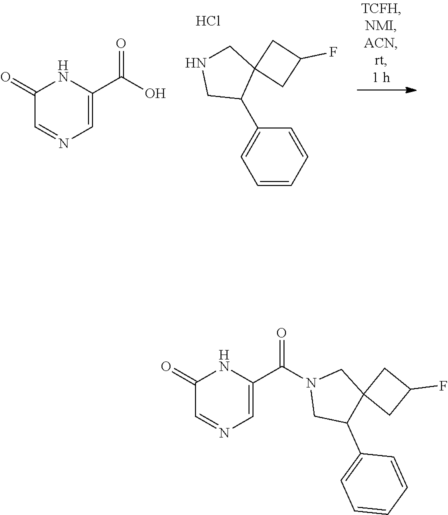 [SMILES: Cl ; O=C(O)c1cncc(=O)[nH]1 ; FC1CC2(CNCC2c2ccccc2)C1 ; O=C(c1cncc(=O)[nH]1)N1CC(c2ccccc2)C2(CC(F)C2)C1]

A solution of 6-oxo-1H-pyrazine-2-carboxylic acid (98 mg, 0.7 mmol), TCFH (400 mg, 1.4 mmol) and NMI (120 mg, 1.4 mmol) in MeCN (2 mL) was stirred for 10 min., then 2-fluoro-8-phenyl-6-azaspiro[3.4]octane (150 mg, 0.7 mmol) was added. The mixture was stirred for 1 h, then filtered, and the filtrate concentrated. The residue was purified by Prep-HPLC (Column: Welch Xtimate C18 ExRS, 250 mm, 10 μm; Mobile Phase: 30-90% MeCN/0.05% aqueous NH₃; Flow rate: 90 mL/min; Wavelength: 254 nm) to afford 6-{2-fluoro-8-phenyl-6-azaspiro[3.4]octane-6-carbonyl}-1H-pyrazin-2-one (95 mg, 41% yield) as a grey solid.

LCMS (ES, m/z): 328 [M+H]⁺

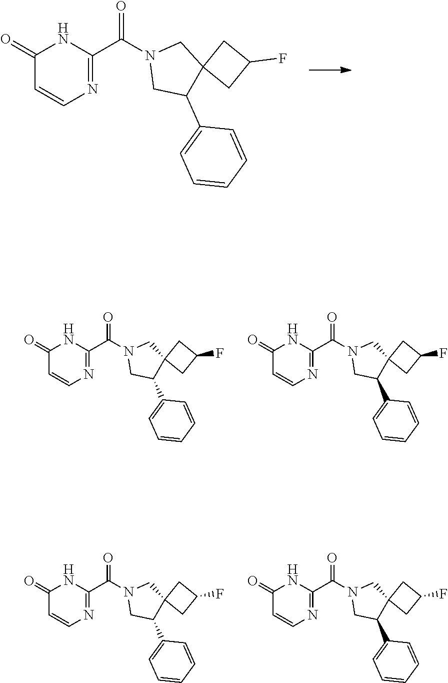 [SMILES: O=C(c1nccc(=O)[nH]1)N1CC(c2ccccc2)C2(CC(F)C2)C1 ; O=C(c1nccc(=O)[nH]1)N1C[C@@H](c2ccccc2)[C@]2(C1)C[C@H](F)C2 ; O=C(c1nccc(=O)[nH]1)N1C[C@H](c2ccccc2)[C@]2(C1)C[C@H](F)C2 ; O=C(c1nccc(=O)[nH]1)N1C[C@@H](c2ccccc2)[C@]2(C1)C[C@@H](F)C2 ; O=C(c1nccc(=O)[nH]1)N1C[C@H](c2ccccc2)[C@]2(C1)C[C@@H](F)C2]

6-{2-Fluoro-8-phenyl-6-azaspiro[3.4]octane-6-carbonyl}-1H-pyrazin-2-one (94 mg) was separated by Prep-HPLC (Column: CHIRALPAKIF-3, 50*4.6 mm, 3 um IF30CB-CM006; Mobile

Phase: EtOH (containing 0.5% FA)/3:1 n-Hexane:DCM; Flow rate: 1.0 mL/min) to afford:

Ex. 25(a) Diastereomer A (First eluting diastereomer) was isolated as an off-white solid (21 mg, 38% yield)

LCMS (ES, m/z): 328 [M+H]⁺, method LCMS14

¹H NMR (300 MHz, DMSO-d₆, ppm) δ 12.05 (br, s, 1H), δ 8.37-8.13 (m, 2H), 7.48-7.06 (m, 5H), 5.28-4.75 (m, 1H), 4.21-3.56 (m, 4H), 3.47-3.32 (m, 1H), 2.46-2.16 (m, 1H), 2.19-1.63 (in, 3H).

Ex. 25(b) Diastereomer B (Second eluting diastereomer) was isolated as an off-white solid (23 mg, 43% yield)

LCMS (ES, m/z): 328 [M+H]⁺, method LCMS14

¹H NMR (300 MHz, DMSO-d₆, ppm) δ 11.96 (br, s, 1H), 8.31-8.07 (m, 2H), 7.51-7.12 (m, 5H), 5.25-4.84 (m, 1H), 4.09-3.53 (m, 4H), 3.47-3.38 (m, 1H), 2.46-2.22 (m, 1H), 2.16-1.69 (m, 3H).

Ex. 25(c) Diastereomer C (Third eluting diastereomer) was isolated as an off-white solid (10 mg, 11% yield)

LCMS (ES, m/z): 328 [M+H]⁺, method LCMS14

¹H NMR (300 MHz, DMSO-d₆, ppm) 8.30-8.19 (m, 1H), 8.19-8.08 (m, 1H), 7.46-7.20 (m, 5H), 4.72-4.24 (m, 1H), 4.13-3.90 (m, 1H), 3.89-3.74 (m, 2H), 3.68 (s, 1H)), 3.57-3.45 (m, 1H), 2.34-1.84 (in, 4H).

Ex. 25(d) Diastereomer 0 (Fourth eluting diastereomer) was isolated as an off-white solid (10 mg, 11%)

LCMS (ES, m/z): 328 [M+H]⁺, method LCMS14

1H NMR (300 MHz, DMSO-d6, ppm) δ 8.34-8.07 (m, 2H), 7.54-7.18 (m, 5H), 4.72-4.30 (m, 1H), 4.09-3.91 (m, 15H), 3.90-3.76 (m, 2H), 3.68 (s, 1H), 3.53-3.38 (m, 1H), 2.42-1.82 (m, 4H).

The Diastereomers A to 0 are (stereochemistry not formally allocated):
(R),(R)-2-(2-fluoro-8-phenyl-6-azaspiro[3.4]octane-6-carbonyl)pyrimidin-4(3H)-one,
(S),(S)-2-(2-fluoro-8-phenyl-6-azaspiro[3.4]octane-6-carbonyl)pyrimidin-4(3H)-one,
(R),(S)-2-(2-fluoro-8-phenyl-6-azaspiro[3.4]octane-6-carbonyl)pyrimidin-4(3H)-one, and
(S),(R)-2-(2-fluoro-8-phenyl-6-azaspiro[3.4]octane-6-carbonyl)pyrimidin-4(3H)-one.

The following compounds were synthesised analogously using the appropriate acid, or equivalent, and amine. Amines were synthesised analogously to previous examples. Enantiomer A corresponds to the eutomer.

TABLE 4

| Example | | | | Separation | Retention |
|---|---|---|---|---|---|
| No | Name and structure | LCMS | ¹H-NMR | conditions | time/min. |
| Example 26(a) and Example 26(b) | 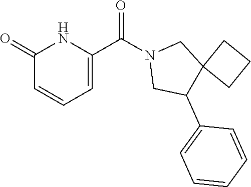 [SMILES: O=C(c1cccc(=O)[nH]1)N1CC(c2ccccc2)C2(CCC2)C1] | Enantiomer A (26a) | | | |
| | 6-(8-phenyl-6-azaspiro[3.4]octane- | 309 | ¹H NMR: (400 | SFC Column: | 6.1 |
| | 6-carbonyl)pyridin-2(1H)-one | [M + H]⁺ | MHz, DMSO-d₆, | CHIRAL ART | |
| | Enantiomer A | | ppm) δ 7.59 (br, | Cellulose-SB | |
| | 6-(8-phenyl-6-azaspiro[3.4]octane- | | s, 1H), 7.41-7.11 | 3*25 cm, 5 μm; | |
| | 6-carbonyl)pyridin-2(1H)-one | | (m, 6H), 6.89- | Mobile Phase: | |
| | Enantiomer B | | 6.61 (m, 1H), | 40% 0.1% NH₃ | |
| | One of Enantiomers A and B is (S)- | | 6.60-6.53 (m, | in MeOH/CO₂; | |
| | 6-(8-phenyl-6-azaspiro[3.4]octane- | | 1H), 4.02-3.46 | Flow rate: 80 | |
| | 6-carbonyl)pyridin-2(1H)-one and | | (m, 4H), 3.29- | mL/min; | |
| | the other is (R)-6-(8-phenyl-6- | | 3.25 (m, 1H), | Wavelength: | |
| | azaspiro[3.4]octane-6- | | 2.14-1.90 (m, | 220 nm | |
| | carbonyl)pyridin-2(1H)-one | | 2H), 1.82-1.58 | | |
| | | | (m, 3H), 1.53- | | |
| | | | 4.48(m, 1H). | | |
| | | Enantiomer B (26b) | | | |
| | | 309 | ¹H NMR: (400 | SFC Column: | 7.1 |
| | | [M + H]⁺ | MHz, DMSO-d₆, | CHIRAL ART | |
| | | | ppm) δ 7.58 (br, | Cellulose-SB | |
| | | | s, 1H), 7.41-7.24 | 3*25 cm, 5 μm; | |
| | | | (m, 5H), 7.20 (d, | Mobile Phase: | |
| | | | J = 7.5 Hz, 1H), | 40% 0.1% NH₃ | |
| | | | 6.89-6.61 (m, | in MeOH/CO₂; | |
| | | | 1H), 6.60-6.54 | Flow rate: 80 | |
| | | | (m, 1H), 4.01- | mL/min; | |
| | | | 3.52 (m, 4H), | Wavelength: | |
| | | | 3.28-3.24 (m, | 220 nm | |
| | | | 1H), 2.05-1.88 | | |
| | | | (m, 2H), 1.83- | | |
| | | | 1.62 (m, 2H), | | |
| | | | 1.53-1.49 (m, | | |
| | | | 2H). | | |
| Example 27(b) and Example 27(a) | 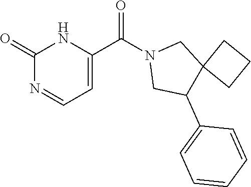 [SMILES: O=C(c1ccnc(=O)[nH]1)N1CC(c2ccccc2)C2(CCC2)C1] | Enantiomer B (27b) | | | |
| | 6-(8-phenyl-6-azaspiro[3.4]octane- | 310 | ¹H NMR: (400 | Column: | 6.2 |
| | 6-carbonyl)pyrimidin-2(1H)-one | [M + H]⁺ | MHz, DMSO-d₆, | CHIRALPAK IA | |
| | Enantiomer B | | ppm) δ 8.11 (dd, | 2*25 cm, 5 μm; | |
| | 6-(8-phenyl-6-azaspiro[3.4]octane- | | J = 10.6, 6.2 Hz, | Mobile Phase: | |
| | 6-carbonyl)pyrimidin-2(1H)-one | | 1H), 7.39- | 30% 1:2 | |
| | Enantiomer A | | 7.35 (m, 2H), | EtOH:MeOH/ | |
| | One of Enantiomers A and B is (S)- | | 7.32-7.18 (m, | Hexane | |
| | 6-(8-phenyl-6-azaspiro[3.4]octane- | | 3H), 6.65-6.51 | (containing | |
| | 6-carbonyl)pyrimidin-2(1H)-one and | | (m, 1H), 3.95- | 0.1% TFA); | |
| | the other is (R)-6-(8-phenyl-6- | | 3.60 (m, 4H), | Flow rate: 25 | |
| | azaspiro[3.4]octane-6- | | 3.35-3.21 (m, | mL/min; | |
| | carbonyl)pyrimidin-2(1H)-one | | 1H), 2.05-1.87 | Wavelength: | |
| | | | (m, 2H), 1.87- | 254 nm | |
| | | | 1.42 (m, 4H). | | |
| | | Enantiomer A (27a) | | | |
| | | 310 | ¹H NMR: (400 | Column: | 9.1 |
| | | [M + H]⁺ | MHz, DMSO-d₆, | CHIRALPAK IA | |
| | | | ppm) δ 8.12 (dd, | 2*25 cm, 5 μm; | |
| | | | J = 10.4, 6.2 Hz, | Mobile Phase: | |
| | | | 1H), 7.39- | 30% 1:2 | |
| | | | 7.35(m, 2H), | EtOH:MeOH/ | |
| | | | 7.30-7.15 (m, | Hexane | |
| | | | 3H), 6.65-6.52 | (containing | |
| | | | (m, 1H), 3.92- | 0.1% TFA); | |
| | | | 3.56 (m, 4H), | Flow rate: 25 | |
| | | | 3.32-3.21 (m, | mL/min; | |
| | | | 1H), 2.06-1.86 | Wavelength: | |
| | | | (m, 2H), 1.86- | 254 nm | |
| | | | 1.45 (m, 4H). | | |
| Example 28(a) and Example 28(b) | 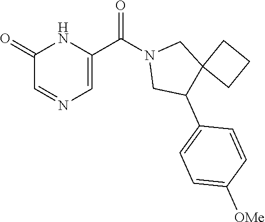 [SMILES: COc1ccc(C2CN(C(=O)c3cncc(=O)[nH]3)CC23CCC3)cc1] | Enantiomer A (28a) | | | |
| | 6-(8-(4-methoxyphenyl)-6- | 340 | ¹H NMR: (400 | Column: | 3.5 |
| | azaspiro[3.4]octane-6- | [M + H]⁺ | MHz, DMSO-d₆, | CHIRALPAK IA | |
| | carbonyl)pyrazin-2(1H)-one | | ppm) δ11.88 (br, | 3*25 cm, 5 μm; | |
| | Enantiomer A | | s, 1H), 8.20 (br, | Mobile Phase: | |
| | 6-(8-(4-methoxyphenyl)-6- | | s, 1H), 8.17 (d, J = | 30% EtOH | |
| | azaspiro[3.4]octane-6- | | 12.4 Hz, 1H), | (containing | |
| | carbonyl)pyrazin-2(1H)-one | | 7.27-7.12 (m, | 0.1% TFA)/3/1 | |
| | Enantiomer B | | 2H), 6.93 (t, J = | Hexane/DCM; | |
| | One of Enantiomers A and B is (S)- | | 8.8 Hz, 2H), | Flow rate: 25 | |
| | 6-(8-(4-methoxyphenyl)-6- | | 4.06-3.51 (m, | mL/min; | |
| | azaspiro[3.4]octane-6- | | 7H), 3.24-3.08 | Wavelength: | |
| | carbonyl)pyrazin-2(1H)-one and the | | (m, 1H), 2.05- | 254 nm | |
| | other is (R)-6-(8-(4- | | 0.87 (m, 6H). | | |
| | methoxyphenyl)-6- | | | | |
| | azaspiro[3.4]octane-6- | Enantiomer B (28b) | | | |
| | carbonyl)pyrazin-2(1H)-one | 340 | ¹H NMR: (400 | Column: | 4.4 |
| | | [M + H]⁺ | MHz, DMSO-d₆, | CHIRALPAK IA | |
| | | | ppm) δ 11.94 (br, | 3*25 cm, 5 μm; | |
| | | | s, 1H), 8.21-8.15 | Mobile Phase: | |
| | | | (m, 2H), 7.3-7.18 | 30% EtOH | |
| | | | (m, 2H), 6.93 (t, J = | (containing | |
| | | | 8.7 Hz, 2H), | 0.1% TFA)/3/1 | |
| | | | 4.06-3.81 (m, | Hexane/DCM; | |
| | | | 1H), 3.81-3.56 | Flow rate: 25 | |
| | | | (m, 6H), 3.22- | mL/min; | |
| | | | 3.17 (m, 1H), | Wavelength: | |
| | | | 2.10-1.36 (m, | 254 nm | |
| | | | 6H). | | |
| Example 29(a) and Example 29(b) | 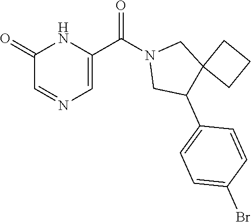 [SMILES: O=C(c1cncc(=O)[nH]1)N1CC(c2ccc(Br)cc2)C2(CCC2)C1] | Enantiomer A (29a) | | | |
| | 6-(8-(4-bromophenyl)-6- | 388, 390 | ¹H NMR: (400 | Column: | 3.5 |
| | azaspiro[3.4]octane-6- | [M + H]⁺ | MHz, DMSO-d₆, | CHIRALPAK IA, | |
| | carbonyl)pyrazin-2(1H)-one | | ppm) δ 11.93 (br, | 2*25 cm, 5 μm; | |
| | Enantiomer A | | s, 1H), 8.30-81.5 | Mobile Phase: | |
| | 6-(8-(4-bromophenyl)-6- | | (m, 2H), 7.56 (t, J = | 30% MeCN | |
| | azaspiro[3.4]octane-6- | | 7.6 Hz, 2H), | (containing | |
| | carbonyl)pyrazin-2(1H)-one | | 7.27-7.19 (m, | 0.1% TFA)/3:1 | |
| | Enantiomer B | | 2H), 4.06-3.88 | Hexane:DCM, | |
| | One of Enantiomers A and B is (S)- | | (m, 1H), 3.88- | Flow rate: 25 | |
| | 6-(8-(4-bromophenyl)-6- | | 3.68 (m, 2H), | mL/min; | |
| | azaspiro[3.4]octane-6- | | 3.67 (s, 1H), | Wavelength: | |
| | carbonyl)pyrazin-2(1H)-one and the | | 3.33-3.26 (m, | 254 nm | |
| | other is (R)-6-(8-(4-bromophenyl)-6- | | 1H), 2.10-1.87 | | |
| | azaspiro[3.4]octane-6- | | (m, 2H), 1.85- | | |
| | carbonyl)pyrazin-2(1H)-one | | 1.40 (m, 4H). | | |
| | | Enantiomer B (29b) | | | |
| | | 388, 390 | ¹H NMR: (400 | Column: | 6.2 |
| | | [M + H]⁺ | MHz, DMSO-d₆, | CHIRALPAK IA, | |
| | | | ppm) δ 8.25-8.17 | 2*25 cm, 5 μm; | |
| | | | (m, 2H), 7.56 (t, J = | Mobile Phase: | |
| | | | 7.7 Hz, 2H), | 30% MeCN | |
| | | | 7.27-7.19 (m, | (containing | |
| | | | 2H), 4.05-3.77 | 0.1% TFA)/3:1 | |
| | | | (m, 1H), 3.80- | Hexane:DCM, | |
| | | | 3.73 (m, 2H), | Flow rate: 25 | |
| | | | 3.67 (s, 1H), | mL/min; | |
| | | | 3.36-3.24 (m, | Wavelength: | |
| | | | 1H), 2.04-1.85 | 254 nm | |
| | | | (m, 2H), 1.84- | | |
| | | | 1.45 (m, 4H). | | |
| Example 30(a) and Example 30(b) | 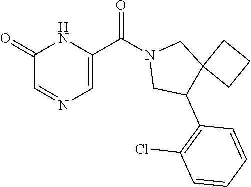 [SMILES: O=C(c1cncc(=O)[nH]1)N1CC(c2ccccc2Cl)C2(CCC2)C1] | Enantiomer A (30a) | | | |
| | 6-(8-(2-chlorophenyl)-6- | 344 | ¹H NMR: (400 | Column: | 3.2 |
| | azaspiro[3.4]octane-6- | [M + H]⁺ | MHz, DMSO-d₆, | CHIRALPAK IA, | |
| | carbonyl)pyrazin-2(1H)-one | | ppm) δ 12.00 (br, | 2*25 cm, 5 μm; | |
| | Enantiomer A | | s, 1H), 8.8.45- | Mobile Phase: | |
| | 6-(8-(2-chlorophenyl)-6- | | 8.10 (m, 2H), | 50% EtOH/ | |
| | azaspiro[3.4]octane-6- | | 7.52 (t, J = 7.5 | Hexane | |
| | carbonyl)pyrazin-2(1H)-one | | Hz, 1H), 7.41- | (containing | |
| | Enantiomer B | | 7.13 (m, 3H), | TFA); | |
| | One of Enantiomers A and B is (S)- | | 4.18-3.57 (m, | 0.1% Flow rate: 25 | |
| | 6-(8-(2-chlorophenyl)-6- | | 5H), 2.2.14-1.99 | mL/min; | |
| | azaspiro[3.4]octane-6- | | (m, 2H), 1.89- | Wavelength: | |
| | carbonyl)pyrazin-2(1H)-one and the | | 1.55 (m, 4H). | 220 nm | |
| | other is (R)-6-(8-(2-chlorophenyl)-6- | Enantiomer B (30b) | | | |
| | azaspiro[3.4]octane-6- | 344 | ¹H NMR: (400 | Column: | 6.0 |
| | carbonyl)pyrazin-2(1H)-one | [M + H]⁺ | MHz, DMSO-d₆, | CHIRALPAK IA, | |
| | | | ppm) δ 11.93 (br, | 2*25 cm, 5 μm; | |
| | | | s, 1H), 8.32 (br, | Mobile Phase: | |
| | | | s, 1H), 8.19 (d, J = | 50% EtOH/ | |
| | | | 14.6 Hz, 1H), | Hexane | |
| | | | 7.52 (t, J = 7.6 | (containing | |
| | | | Hz, 1H), 7.44- | 0.1% TFA); | |
| | | | 7.16 (m, 3H), | Flow rate: 25 | |
| | | | 4.13-3.84 (m, | mL/min; | |
| | | | 3H), 3.83-3.76 | Wavelength: | |
| | | | (m, 1H), 3.74- | 220 nm | |
| | | | 3.66 (m, 1H), | | |
| | | | 2.14-1.92 (m, | | |
| | | | 2H), 1.90-1.53 | | |
| | | | (m, 4H). | | |
| Example 31(a) and Example 31(b) | 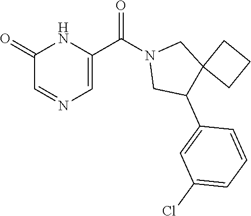 [SMILES: O=C(c1cncc(=O)[nH]1)N1CC(c2cccc(Cl)c2)C2(CCC2)C1] | Enantiomer A (31a) | | | |
| | 6-(8-(3-chlorophenyl)-6- | 344 | ¹H NMR: (400 | Column: | 6.5 |
| | azaspiro[3.4]octane-6- | [M + H]⁺ | MHz, DMSO-d₆, | CHIRAL ART | |
| | carbonyl)pyrazin-2(1H)-one | | ppm) δ 8.40 (br, | Cellulose-SB, | |
| | Enantiomer A | | s, 1H), 8.19 (d, J = | 3*25 cm, 5 μm; | |
| | 6-(8-(3-chlorophenyl)-6- | | 12.1 Hz, 1H), | Mobile Phase: | |
| | azaspiro[3.4]octane-6- | | 7.48-7.30 (m, | 10% EtOH | |
| | carbonyl)pyrazin-2(1H)-one | | 3H), 7.23 (dd, J = | (containing | |
| | Enantiomer B | | 22.6, 7.5 Hz, | 0.1% TFA)/3:1 | |
| | One of Enantiomers A and B is (S)- | | 1H), 4.09-3.88 | Hexane:DCM; | |
| | 6-(8-(3-chlorophenyl)-6- | | (m, 1H), 3.89- | Flow rate: 35 | |
| | azaspiro[3.4]octane-6- | | 3.73 (m, 2H), | mL/min; | |
| | carbonyl)pyrazin-2(1H)-one and the | | 3.68 (s, 1H), | Wavelength: | |
| | other is (R)-6-(8-(3-chlorophenyl)-6- | | 3.37-3.28 (m, | 254 nm | |
| | azaspiro[3.4]octane-6- | | 1H), 2.09-1.87 | | |
| | carbonyl)pyrazin-2(1H)-one | | (m, 2H), 1.85- | | |
| | | | 1.39 (m, 4H). | | |
| | | Enantiomer B (31b) | | | |
| | | 344 | ¹H NMR: (400 | Column: | 7.7 |
| | | [M + H]⁺ | MHz, DMSO-d₆, | CHIRAL ART | |
| | | | ppm) δ 8.26 (br, | Cellulose-SB, | |
| | | | s, 1H), 8.19 (d, J = | 3*25 cm, 5 μm; | |
| | | | 12.1 Hz, 1H), | Mobile Phase: | |
| | | | 7.48-7.30 (m, | 10% EtOH | |
| | | | 3H), 7.23 (dd, J= | (containing | |
| | | | 22.7, 7.5 Hz, | 0.1% TFA)/3:1 | |
| | | | 1H), 4.02-3.81 | Hexane:DCM; | |
| | | | (m, 2H), 3.81- | Flow rate: 35 | |
| | | | 3.72 (m, 1H), | mL/min; | |
| | | | 3.68 (s, 1H), 3.33 | Wavelength: | |
| | | | (q, J = 6.0 Hz, | 254 nm | |
| | | | 1H), 2.04-1.88 | | |
| | | | (m, 2H), 1.88- | | |
| | | | 1.43 (m, 4H). | | |
| Example 32(a) and Example 32(b) | 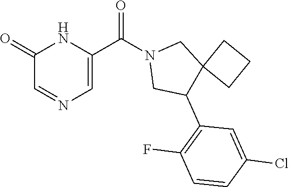 [SMILES: O=C(c1cncc(=O)[nH]1)N1CC(c2cc(Cl)ccc2F)C2(CCC2)C1] | Enantiomer A (32a) | | | |
| | 6-8-(5-chloro-2-fluorophenyl)-6- | | | | |
| | azaspiro[3.4]octane-6- | 362 | ¹H NMR (400 | Column: | 4.2 |
| | carbonyl)pyrazin-2(1H)-one | [M + H]⁺ | MHz, DMSO-d₆, | CHIRALPAK IA, | |
| | Enantiomer A | | ppm) δ 8.29-8.10 | 2*25 cm, 5 μm; | |
| | 6-8-(5-chloro-2-fluorophenyl)-6- | | (m, 2H), 7.45- | Mobile Phase: | |
| | azaspiro[3.4]octane-6- | | 7.40 (m, 1H), | 40% EtOH/ | |
| | carbonyl)pyrazin-2(1H)-one | | 7.36-7.24 (m, | Hexane | |
| | Enantiomer B | | 2H), 4.08-3.91 | (containing | |
| | One of Enantiomers A and B is (S)- | | (m, 1H), 3.88- | 0.2% FA); Flow | |
| | 6-8-(5-chloro-2-fluorophenyl)-6- | | 3.78 (m, 2H), | rate: 25 mL/min; | |
| | azaspiro[3.4]octane-6- | | 3.70 (d, J = 6.1 | Wavelength: | |
| | carbonyl)pyrazin-2(1H)-one and the | | Hz, 1H), 3.68- | 254 nm | |
| | other is (R)-6- 8-(5-chloro-2- | | 3.60 (m, 1H), | | |
| | fluorophenyl)-6- | | 2.05-1.90 (m, | | |
| | azaspiro[3.4]octane-6- | | 2H), 1.81-1.55 | | |
| | carbonyl)pyrazin-2(1H)-one | | (m, 4H). | | |
| | | Enantiomer B (32b) | | | |
| | | 362 | ¹H NMR (400 | Column: | 6.3 |
| | | [M + H]⁺ | MHz, DMSO-d₆, | CHIRALPAK IA, | |
| | | | ppm) δ 8.35-8.12 | 2*25 cm, 5 μm; | |
| | | | (m, 2H), 7.50- | Mobile Phase: | |
| | | | 7.35 (m, 1H), | 40% EtOH/ | |
| | | | 7.36-7.21 (m, | Hexane | |
| | | | 2H), 4.04-3.92 | (containing | |
| | | | (m, 1H), 3.89- | 0.2% FA); Flow | |
| | | | 3.72 (m, 2H), | rate: 25 mL/min; | |
| | | | 3.70 (d, J = 5.3 | Wavelength: | |
| | | | Hz, 1H), 3.65- | 254 nm | |
| | | | 3.60 (m, 1H), | | |
| | | | 2.05-1.90 (m, | | |
| | | | 2H), 1.87-1.55 | | |
| | | | (m, 4H). | | |
| | | Enantiomer A (33a) | | | |
| Example 33(a) and Example 33(b) | 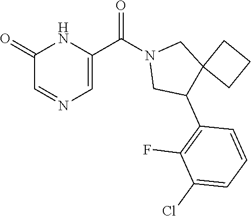 [SMILES: O=C(c1cncc(=O)[nH]1)N1CC(c2cccc(Cl)c2F)C2(CCC2)C1] | 362 [M + H]⁺ | ¹H NMR: (400 MHz, DMSO-d₆, ppm) δ 8.27-8.09 (m, 2H), 7.56- 7.47 (m, 1H), 7.31-7.14 (m, 2H), 4.08-3.80 (m, 2H), 3.76- 3.57 (m, 3H), 2.11-1.90 (m, 2H), 1.88-1.55 (m, 4H). | Column: CHIRALPAKIG- 3, 50*4.6 mm, 3 um IG30CB- BW008; Mobile Phase: 30% EtOH (containing 0.5% FA)/5:1 n-Hexane:DCM; Flow rate: 1.0 mL/min | First eluting enantiomer |
| | 6-(8-(3-chloro-2-fluorophenyl)-6- | Enantiomer B (33b) | | | |
| | azaspiro[3.4]octane-6- | 362 | ¹H NMR: (400 | Column: | Second |
| | carbonyl)pyrazin-2(1H)-one | [M + H]⁺ | MHz, DMSO-d₆, | CHIRALPAKIG- | eluting |
| | Enantiomer A | | ppm) δ 8.27- | 3, 50*4.6 mm, | enantiomer |
| | 6-(8-(3-chloro-2-fluorophenyl)-6- | | 8.09 (m, 2H), | 3 um IG30CB- | |
| | azaspiro[3.4]octane-6- | | 7.57-7.47 (m, | BW008; Mobile | |
| | carbonyl)pyrazin-2(1H)-one | | 1H), 7.32-7.14 | Phase: 30% | |
| | Enantiomer B | | (m, 2H), 4.09- | EtOH | |
| | One of Enantiomers A and B is (S)- | | 3.80 (m, 2H), | (containing | |
| | 6-(8-(3-chloro-2-fluorophenyl)-6- | | 3.80-3.57 (m, | 0.5% FA)/5:1 | |
| | azaspiro[3.4]octane-6- | | 3H), 2.11-1.87 | n-Hexane:DCM; | |
| | carbonyl)pyrazin-2(1H)-one and the | | (m, 2H), 1.85- | Flow rate: 1.0 | |
| | other is (R)-6-(8-(3-chloro-2- | | 1.57 (m, 4H). | mL/min | |
| | fluorophenyl)-6- | | | | |
| | azaspiro[3.4]octane-6- | | | | |
| | carbonyl)pyrazin-2(1H)-one | | | | |
| Example 34(a) and Example 34(b) | 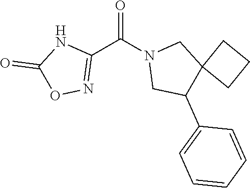 [SMILES: O=C(c1noc(=O)[nH]1)N1CC(c2ccccc2)C2(CCC2)C1] | Enantiomer A (34a) | | | |
| | 3-(8-phenyl-6-azaspiro[3.4]octane- | 300 | ¹H NMR: (400 | Reverse-phase | N/A |
| | 6-carbonyl)-1,2,4-oxadiazol-5(4H)- | [M + H]⁺ | MHz, DMSO-d₆, | flash | |
| | one Enantiomer A | | ppm) δ 13.07 (br, | chromatography | |
| | 3-(8-phenyl-6-azaspiro[3.4]octane- | | s, 1H), 7.38-7.36 | (Column, C18 | |
| | 6-carbonyl)-1,2,4-oxadiazol-5(4H)- | | (m, 2H), 7.33- | silica gel; mobile | |
| | one Enantiomer B | | 7.24 (m, 3H), | phase, 10% to | |
| | One of Enantiomers A and B is (S)- | | 4.15-4.02 (m, | 50% MeCN in | |
| | 3-(8-phenyl-6-azaspiro[3.4]octane- | | 1H), 3.92 (d, J = | water (0.1% | |
| | 6-carbonyl)-1,2,4-oxadiazol-5(4H)- | | 4.9 Hz, 1H), | FA); Detector, | |
| | one and the other is (R)-3-(8- | | 3.89-3.81 (m, | UV 254 nm) | |
| | phenyl-6-azaspiro[3.4]octane-6- | | 1H), 3.77 (dd, J = | Enantiomers | |
| | carbonyl)-1,2,4-oxadiazol-5(4H)- | | 12.8, 6.0 Hz, | had been | |
| | one | | 1H), 3.31-3.25 | separated at a | |
| | | | (m, 1H), 2.03- | previous step | |
| | | | 1.95 (m, 2H), | | |
| | | | 1.86-1.72 (m, | | |
| | | | 2H), 1.68-1.44 | | |
| | | | (m, 2H). | | |
| | | Enantiomer B (34b) | | | |
| | | 300 | ¹H NMR: (400 | Reverse-phase | N/A |
| | | [M + H]⁺ | MHz, DMSO-d₆, | flash | |
| | | | ppm) δ 13.06 (br, | chromatography | |
| | | | s, 1H), 7.39-7.36 | (Column, C18 | |
| | | | (m, 2H), 7.32- | silica gel; mobile | |
| | | | 7.23 (m, 3H), | phase, 10% to | |
| | | | 4.13-4.00 (m, | 50% MeCN in | |
| | | | 1H), 3.90 (d, J = | water (0.1% | |
| | | | 3.3 Hz, 1H), | FA); Detector, | |
| | | | 3.88-3.80 (m, | UV 254 nm) | |
| | | | 1H), 3.79-3.69 | Enantiomers | |
| | | | (m, 1H), 3.31- | had been | |
| | | | 3.25 (m, 1H), | separated at a | |
| | | | 2.00-1.97 (m, | previous step | |
| | | | 2H), 1.87-1.70 | | |
| | | | (m, 2H), 1.68- | | |
| | | | 1.44 (m, 2H). | | |

### Example 35(a) 6-(8-(4-fluorophenyl)-6-azaspiro[3.4]octane-6-carbonyl)pyrazin-2(1H)-one Enantiomer A and Example 35(b) 6-(8-(4-fluorophenyl)-6-azaspiro[3.4]octane-6-carbonyl)pyrazin-2(1H)-one Enantiomer B

### Step 1. Preparation of 1-[1-(4-Fluorophenyl)-2-nitroethyl]cyclobutane-1-carbaldehyde

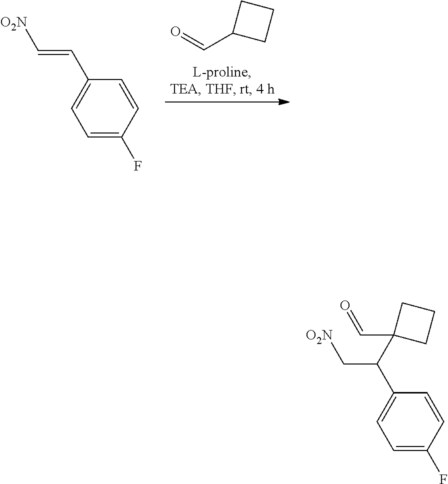 [SMILES: O=[N+]([O-])/C=C/c1ccc(F)cc1 ; O=CC1CCC1 ; O=CC1(C(C[N+](=O)[O-])c2ccc(F)cc2)CCC1]

To a stirred mixture of styrene, (E)-1-fluoro-4-(2-nitrovinyl)benzene (6.0 g, 36 mmol) and L-praline (4.1 g, 36 mmol, 1 eq) in THF (60 mL) were added cyclobutanecarbaldehyde (9.1 g, 110 mmol, 3 eq) and Et₃N (1.1 g, 11 mmol, 0.3 eq). The resulting mixture was stirred for 4 h then concentrated. The residue was purified by silica gel column chromatography, eluting with petroleum ether/EtOAc (5/1) to afford 1-[1-(4-fluorophenyl)-2-nitroethyl]cyclobutane-1-carbaldehyde (4.6 g, 50% yield) as a light yellow oil.

LCMS (ES, m/z): 250 [M−H]⁻

### Step 2. Preparation of 6-Oxo-1H-pyrazine-2-carboxylic acid

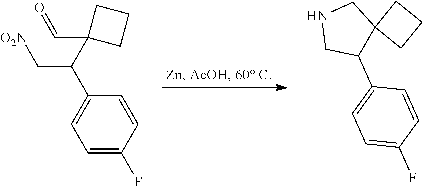 [SMILES: O=CC1(C(C[N+](=O)[O-])c2ccc(F)cc2)CCC1 ; Fc1ccc(C2CNCC23CCC3)cc1]

To 1-[1-(4-fluorophenyl)-2-nitroethyl]cyclobutane-1-carbaldehyde (0.85 g, 3.4 mmol) in EtOH (17 mL) was added zinc powder (1.1 g, 17 mmol, 5 eq) and acetic acid (1.6 g, 27 mmol, 8 eq). The reaction mixture was stirred for 2 h at 60° C., then cooled and filtered. The filtrate was concentrated, and the residue taken up in 4M HCl in MeOH (20 mL). This was stirred for 2 h, then concentrated. The residue was triturated with EtOAc and petroleum ether to afford 6-[8-(4-fluorophenyl)-6-azaspiro[3.4]octane-6-carbonyl]-1H-pyrazin-2-one (0.6 g, 86% yield) as a white solid.

LCMS (ES, m/z): 206 [M+H]⁺

### Step 3. Preparation of 6-[8-(4-Fluorophenyl)-6-azaspiro[3.4]octane-6-carbonyl]-1H-pyrazin-2-one

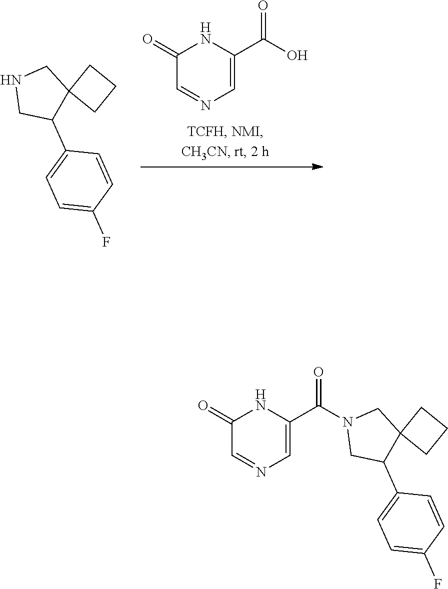 [SMILES: Fc1ccc(C2CNCC23CCC3)cc1 ; O=C(O)c1cncc(=O)[nH]1 ; O=C(c1cncc(=O)[nH]1)N1CC(c2ccc(F)cc2)C2(CCC2)C1]

To 6-oxo-1H-pyrazine-2-carboxylic acid (0.20 g, 1.4 mmol) in MeCN (2 mL) was added TCFH (0.6 g, 2.1 mmol, 1.5 eq) and NMI (0.29 g, 3.6 mmol, 2.5 eq). The reaction mixture was stirred for 15 min. then 8-(4-fluorophenyl)-6-azaspiro[3.4]octane (0.35 g, 1.7 mmol, 1.2 eq) was added. The resulting mixture was stirred for 1 h, then filtered, and the filtrate concentrated. The residue was purified by reverse-phase flash chromatography with the following conditions: (column, C18 silica gel; mobile phase: 10-50% MeOH/0.1% aqueous NH₃·H₂O over 10 min.; detector, UV 254 nm) to afford 6-[8-(4-fluorophenyl)-6-azaspiro[3.4]octane-6-carbonyl]-1H-pyrazin-2-one (120 mg, 25% yield) as an off-white solid.

LCMS (ES, m/z): 328 [M+H]⁺

### Step 4. Preparation of 6-(8-(4-fluorophenyl)-6-azaspiro[3.4]octane-6-carbonyl)pyrazin-2(1H)-one Enantiomer A and 6-(8-(4-fluorophenyl)-6-azaspiro[3.4]octane-6-carbonyl)pyrazin-2(1H)-one Enantiomer B

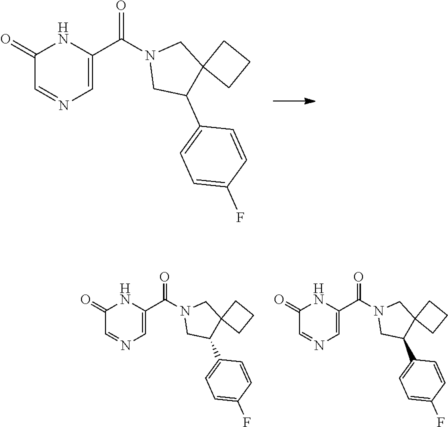

6-[8-(4-Fluorophenyl)-6-azaspiro[3.4]octane-6-carbonyl]-1H-pyrazin-2-one (120 mg, 0.4 mmol) was separated by chromatography (Column: XA-CHIRALPAK IG, 3×25 cm, 5 μm; Mobile Phase: 20% EtOH (containing 0.1% diethylamine)/Hexane; Flow rate: 35 mL/min; Wave Length: 254 nm) to afford:

Ex. 35(a) 6-[-8-(4-Fluorophenyl)-6-azaspiro[3.4]octane-6-carbonyl]-1H-pyrazin-2-one Enantiomer A (27 mg, 22% yield) as an off-white solid.

Rt=10.3 min.

LCMS (ES, m/z): 328 [M+H]⁺

¹H NMR: (400 MHz, DMSO-d₆, ppm) δ 12.01 (br, s, 1H), 8.35-8.15 (m, 2H), 7.37-7.25 (m, 2H), 7.22-7.16 (m, 2H), 4.05-3.60 (m, 4H), 3.30-3.27 (m, 1H), 2.05-1.87 (m, 2H), 1.86-1.44 (m, 4H).

Ex.35(b) 6-[8-(4-fluorophenyl)-6-azaspiro[3.4]octane-6-carbonyl]-1H-pyrazin-2-one Enantiomer B (24 mg, 20% yield) as an off-white solid.

Rt=12.1 min.

LC-MS (ES, m/z): 328 [M+H]⁺

¹H NMR: (400 MHz, DMSO-d₆, ppm) δ 11.98 (br, s, 1H), 8.32-8.17 (m, 2H), 7.37-7.24 (m, 2H), 7.22-7.18 (m, 2H), 4.04-3.62 (m, 4H), 3.33-3.27 (m, 1H), 2.05-1.88 (m, 2H), 1.86-1.44 (m, 4H).

One of Enantiomers A and B is (S)-6-(8-(4-fluorophenyl)-6-azaspiro[3.4]octane-6-carbonyl)pyrazin-2(1H)-one and the other is (R)-6-(8-(4-fluorophenyl)-6-azaspiro[3.4]octane-6-carbonyl)pyrazin-2(1H)-one.

The following compounds were synthesised analogously using the appropriate acid, or equivalent, and amine. Amines were synthesised analogously to previous examples. Enantiomer A corresponds to the eutomer.

TABLE 5

| Example | | | | Separation | Retention |
|---|---|---|---|---|---|
| No | Name and structure | LCMS | ¹H-NMR | conditions | time/min. |
| Example 36(b) and Example 36(a) | 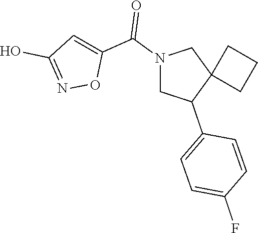 [SMILES: O=C(c1cc(O)no1)N1CC(c2ccc(F)cc2)C2(CCC2)C1] | Enantiomer B (36b) | | | |
| | (8-(4-fluorophenyl)-6- | 317 | ¹H NMR: (400 | Column: XA- | 5.6 |
| | azaspiro[3.4]octan-6-yl)(3- | [M + H]⁺ | MHz, DMSO-d₆, | CHIRALPAK | |
| | hydroxyisoxazol-5- | | ppm) δ 11.75 (br, | OJ-H, 2*25 cm, | |
| | yl)methanone | | s, 1H), 7.37-7.28 | 5 μm; Mobile | |
| | Enantiomer B | | (m, 2H), 7.19 (t, J = | Phase: 20% | |
| | (8-(4-fluorophenyl)-6- | | 8.8 Hz, 2H), | EtOH/Hexane; | |
| | azaspiro[3.4]octan-6-yl)(3- | | 6.60 (s, 1H), 4.09- | Flow rate: 25 | |
| | hydroxyisoxazol-5- | | 3.93 (m, 1H), | mL/min; | |
| | yl)methanone | | 3.89-3.58 (m, 3H), | Wavelength: | |
| | Enantiomer A | | 3.38-3.34 (m, 1H), | 254 nm | |
| | One of Enantiomers A and B | | 2.03-1.91 (m, 2H), | | |
| | is (S)-(8-(4-fluorophenyl)-6- | | 1.85-1.71 (m, 2H), | | |
| | azaspiro[3.4]octan-6-yl)(3- | | 1.68-1.58 (m, 1H), | | |
| | hydroxyisoxazol-5- | | 1.56-1.38 (m, 1H). | | |
| | yl)methanone and the other is | Enantiomer A (36a) | | | |
| | (R)-(8-(4-fluorophenyl)-6- | 317 | ¹H NMR: (400 | Column: XA- | 8.0 |
| | azaspiro[3.4]octan-6-yl)(3- | [M + H]⁺ | MHz, DMSO-d₆, | CHIRALPAK | |
| | hydroxyisoxazol-5- | | ppm) δ 11.74 (br, | OJ-H, 2*25 cm, | |
| | yl)methanone | | s, 1H), 7.38-7.25 | 5 μm; Mobile | |
| | | | (m, 2H), 7.24-7.12 | Phase: 20% | |
| | | | (m, 2H), 6.59 (s, | EtOH/Hexane; | |
| | | | 1H), 4.10-3.90 (m, | Flow rate: 25 | |
| | | | 1H), 3.88-3.57 (m, | mL/min; | |
| | | | 3H), 3.32-3.30 (m, | Wavelength: | |
| | | | 1H), 2.04-1.90 (m, | 254 nm | |
| | | | 2H), 1.86-1.69 (m, | | |
| | | | 2H), 1.70-1.55 (m, | | |
| | | | 1H), 1.57-1.33 (m, | | |
| | | | 1H). | | |
| Example 37(a) and Example 37(b) | 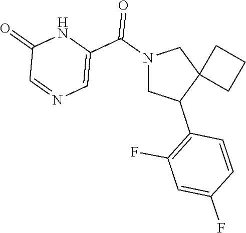 [SMILES: O=C(c1cncc(=O)[nH]1)N1CC(c2ccc(F)cc2F)C2(CCC2)C1] | Enantiomer A (37a) | | | |
| | 6-(8-(2,4-difluorophenyl)-6- | 346 | ¹H NMR (400 | Column: XA | 8.2 |
| | azaspiro[3.4]octane-6- | [M + H]⁺ | MHz, DMSO-d₆, | CHIRALPAK IA, | |
| | carbonyl)pyrazin-2(1H)-one | | ppm) δ 8.31-8.09 | 2*25 cm, 5 μm; | |
| | Enantiomer A | | (m, 2H), 7.37-7.16 | Mobile Phase: | |
| | 6-(8-(2,4-difluorophenyl)-6- | | (m, 2H), 7.12-7.08 | 20% EtOH | |
| | azaspiro[3.4]octane-6- | | (m, 1H), 4.01-3.65 | (containing | |
| | carbonyl)pyrazin-2(1H)-one | | (m, 4H), 3.61-3.54 | 0.1% FA)/ | |
| | Enantiomer B | | (m, 1H), 2.03-1.48 | Hexane; Flow | |
| | One of Enantiomers A and B | | (m, 6H). | rate: 25 mL/min; | |
| | is (S)-6-(8-(2,4- | | | Wavelength: | |
| | difluorophenyl)-6- | | | 254 nm | |
| | azaspiro[3.4]octane-6- | Enantiomer B (37b) | | | |
| | carbonyl)pyrazin-2(1H)-one | 346 | ¹H NMR (400 | Column: XA | 14.5 |
| | and the other is (R)-6-(8-(2,4- | [M + H]⁺ | MHz, DMSO-d₆, | CHIRALPAK IA, | |
| | difluorophenyl)-6- | | ppm) δ 12.19 (brs, | 2*25 cm, 5 μm; | |
| | azaspiro[3.4]octane-6- | | 1H), 8.38-8.09 (m, | Mobile Phase: | |
| | carbonyl)pyrazin-2(1H)-one | | 2H), 7.43-7.20 (m, | 20% EtOH | |
| | | | 2H), 7.13-7.06 (m, | (containing | |
| | | | 1H), 4.08-3.48 (m, | 0.1% FA)/ | |
| | | | 5H), 2.01-1.44 (m, | Hexane; Flow | |
| | | | 6H). | rate: 25 mL/min; | |
| | | | | Wavelength: | |
| | | | | 254 nm | |
| Example 38(b) and Example 38(a) | 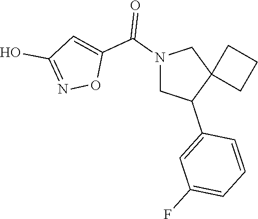 [SMILES: O=C(c1cc(O)no1)N1CC(c2cccc(F)c2)C2(CCC2)C1] | Enantiomer B (38b) | | | |
| | (8-(3-fluorophenyl)-6- | 317 | ¹H NMR (300 | Column: XA | 4.5 |
| | azaspiro[3.4]octan-6-yl)(3- | [M + H]⁺ | MHz, DMSO-d₆, | CHIRALPAK IA, | |
| | hydroxyisoxazol-5- | | ppm) δ 11.81 (br, | 2*25 cm, 5 μm; | |
| | yl)methanone | | s, 1H), 7.49-7.33 | Mobile Phase: | |
| | Enantiomer B | | (m, 1H), 7.24-7.09 | 20% EtOH | |
| | (8-(3-fluorophenyl)-6- | | (m, 3H), 6.59 (s, | (containing | |
| | azaspiro[3.4]octan-6-yl)(3- | | 1H), 4.20-3.96 (m, | 0.1% FA)/ | |
| | hydroxyisoxazol-5- | | 1H), 3.94-3.77 (m, | Hexane; Flow | |
| | yl)methanone | | 2H), 3.77-3.56 (m, | rate: 25 mL/min; | |
| | Enantiomer A | | 1H), 3.44-3.35 (m, | Wavelength: | |
| | One of Enantiomers A and B | | 1H), 2.10-1.94 (m, | 254 nm | |
| | is (S)-(8-(3-fluorophenyl)-6- | | 2H), 1.90-1.71 (m, | | |
| | azaspiro[3.4]octan-6-yl)(3- | | 2H), 1.70-1.38 (m, | | |
| | hydroxyisoxazol-5- | | 2H). | | |
| | yl)methanone and the other is | Enantiomer A (38a) | | | |
| | (R)-(8-(3-fluorophenyl)-6- | 317 | ¹H NMR (300 | Column: XA | 8.0 |
| | azaspiro[3.4]octan-6-yl)(3- | [M + H]⁺ | MHz, DMSO-d₆, | CHIRALPAK IA, | |
| | hydroxyisoxazol-5- | | ppm) δ 11.77 (br, | 2*25 cm, 5 μm; | |
| | yl)methanone | | s, 1H), 7.51-7.35 | Mobile Phase: | |
| | | | (m, 1H), 7.24-7.07 | 20% EtOH | |
| | | | (m, 3H), 6.60 (s, | (containing | |
| | | | 1H), 4.16-3.96 (m, | 0.1% FA)/ | |
| | | | 1H), 3.86 (s, 1H), | Hexane; Flow | |
| | | | 3.84-3.56 (m, 2H), | rate: 25 mL/min; | |
| | | | 3.41-3.37 (m, 1H), | Wavelength: | |
| | | | 2.10-1.94 (m, 2H), | 254 nm | |
| | | | 1.89-1.72 (m, 2H), | | |
| | | | 1.71-1.40 (m, 2H). | | |
| Example 39(b) and Example 39(a) | 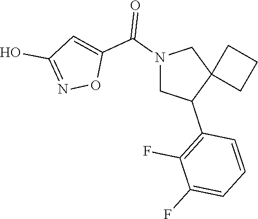 [SMILES: O=C(c1cc(O)no1)N1CC(c2cccc(F)c2F)C2(CCC2)C1] | Enantiomer B (39b) | | | |
| | (8-(2,3-difluorophenyl)-6- | 335 | ¹H NMR (300 | Column: XA- | 9.5 |
| | azaspiro[3.4]octan-6-yl)(3- | [M + H]⁺ | MHz, DMSO-d₆, | CHIRAL ART | |
| | hydroxyisoxazol-5- | | ppm) δ 11.81 (br, | Cellulose-SZ, | |
| | yl)methanone | | s, 1H), 7.38- | 3*25 cm 5 um; | |
| | Enantiomer B | | 7.33(m, 1H), 7.31- | Mobile Phase A: | |
| | (8-(2,3-difluorophenyl)-6- | | 7.18 (m, 1H), | 20% IPA/ | |
| | azaspiro[3.4]octan-6-yl)(3- | | 7.15-7.10 (m, 1H), | Hexane | |
| | hydroxyisoxazol-5- | | 6.59 (d, J = 5.8 | (containing | |
| | yl)methanone | | Hz, 1H), 4.15-4.10 | 0.2% FA); Flow | |
| | Enantiomer A | | (m, 1H), 4.03-3.61 | rate: 35 mL/min; | |
| | One of Enantiomers A and B | | (m, 4H), 2.05-2.00 | Wavelength: | |
| | is (S)-(8-(2,3-difluorophenyl)- | | (m, 2H), 1.90-1.56 | 254 nm | |
| | 6-azaspiro[3.4]octan-6-yl)(3- | | (m, 4H). | | |
| | hydroxyisoxazol-5- | Enantiomer A (39a) | | | |
| | yl)methanone and the other is | 335 | ¹H NMR (300 | Column: XA- | 11.7 |
| | (R)-(8-(2,3-difluorophenyl)-6- | [M + H]⁺ | MHz, DMSO-d₆, | CHIRAL ART | |
| | azaspiro[3.4]octan-6-yl)(3- | | ppm) δ 11.81 (br, | Cellulose-SZ, | |
| | hydroxyisoxazol-5- | | s, 1H), 7.40-7.35 | 3*25 cm 5 um; | |
| | yl)methanone | | (m, 1H), 7.31-7.18 | Mobile Phase A: | |
| | | | (m, 1H), 7.15-7.09 | 20% IPA/ | |
| | | | (m, 1H), 6.59 (d, J = | Hexane | |
| | | | 5.8 Hz, 1H), | (containing | |
| | | | 4.15-3.61 (m, 5H), | 0.2% FA); Flow | |
| | | | 2.05-2.00 (m, 2H), | rate: 35 mL/min; | |
| | | | 1.90-1.56 (m, 4H). | Wavelength: | |
| | | | | 254 nm | |
| Example 40(b) and Example 40(a) | 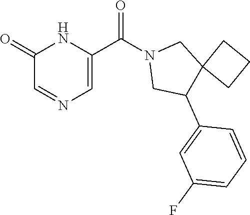 [SMILES: O=C(c1cncc(=O)[nH]1)N1CC(c2cccc(F)c2)C2(CCC2)C1] | Enantiomer B (40b) | | | |
| | 6-(8-(3-fluorophenyl)-6- | 328 | ¹H NMR (300 | Column: XA- | 7.0 |
| | azaspiro[3.4]octane-6- | [M + H]⁺ | MHz, DMSO-d₆, | CHIRALPAK IF, | |
| | carbonyl)pyrazin-2(1H)-one | | ppm) δ 12.76 (br, | 3*25 cm, 5 μm; | |
| | Enantiomer B | | s, 1H), 8.33-8.04 | Mobile Phase: | |
| | 6-(8-(3-fluorophenyl)-6- | | (m, 2H), 7.47-7.32 | 30% EtOH | |
| | azaspiro[3.4]octane-6- | | (m, 1H), 7.21-7.02 | (containing | |
| | carbonyl)pyrazin-2(1H)-one | | (m, 3H), 4.05-3.80 | 0.1% FA)/ACN, | |
| | Enantiomer A | | (m, 3H), 3.79-3.76 | Flow rate: 35 | |
| | One of Enantiomers A and B | | (m, 1H), 3.66 (s, | mL/min; | |
| | is (S)- 6-(8-(3-fluorophenyl)-6- | | 1H), 2.08-1.86 (m, | Wavelength: | |
| | azaspiro[3.4]octane-6- | | 2H), 1.86-1.60 (m, | 254 nm | |
| | carbonyl)pyrazin-2(1H)-one | | 2H), 1.63-1.44 (m, | | |
| | and the other is (R)-6-(8-(3- | | 2H). | | |
| | fluorophenyl)-6- | Enantiomer A (40a) | | | |
| | azaspiro[3.4]octane-6- | 328 | ¹H NMR (300 | Column: XA- | 9.4 |
| | carbonyl)pyrazin-2(1H)-one | [M + H]⁺ | MHz, DMSO-d₆, | CHIRALPAK IF, | |
| | | | ppm) δ 8.29-8.10 | 3*25 cm, 5 μm; | |
| | | | (m, 2H), 7.49-7.35 | Mobile Phase: | |
| | | | (m, 1H), 7.18-7.05 | 30% EtOH | |
| | | | (m, 3H), 4.05-3.95 | (containing | |
| | | | (m, 1H), 3.92-3.81 | 0.1% FA)/ACN, | |
| | | | (m, 2H), 3.79-3.75 | Flow rate: 35 | |
| | | | (m, 1H), 3.67 (s, | mL/min; | |
| | | | 1H), 2.11-1.88 (m, | Wavelength: | |
| | | | 2H), 1.85-1.64 (m, | 254 nm | |
| | | | 2H), 1.63-1.45 (m, | | |
| | | | 2H). | | |
| Example 41(a) and Example 41(b) | 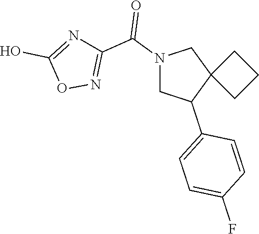 [SMILES: O=C(c1noc(O)n1)N1CC(c2ccc(F)cc2)C2(CCC2)C1] | Enantiomer A (41a) | | | |
| | 3-(8-(4-fluorophenyl)-6- | 318 | ¹H NMR: (400 | Reverse-phase | N/A |
| | azaspiro[3.4]octane-6- | [M + H]⁺ | MHz, DMSO-d₆, | flash | |
| | carbonyl)-1,2,4-oxadiazol- | | ppm) δ 13.04 (s, | chromatography: | |
| | 5(4H)-one | | 1H), 7.33-7.26 (m, | Column, C18 | |
| | Enantiomer A | | 2H), 7.23-7.16 (m, | silica getl; Mobile | |
| | 3-(8-(4-fluorophenyl)-6- | | 2H), 4.13-3.97 (m, | Phase, 10% to | |
| | azaspiro[3.4]octane-6- | | 1H), 3.90 (d, J = | 50% MeCN in | |
| | carbonyl)-1,2,4-oxadiazol- | | 7.2 Hz, 1H), 3.87- | water | |
| | 5(4H)-one | | 3.68 (m, 1H), 3.65 | containing | |
| | Enantiomer B | | (d, J= 4.6 Hz, 1H), | 0.1% HCl; | |
| | One of Enantiomers A and B | | 3.36-3.32 (m, 1H), | detector, UV | |
| | is (S)-3-(8-(4-fluorophenyl)-6- | | 2.01-1.90 (m, 2H), | 254 nm | |
| | azaspiro[3.4]octane-6- | | 1.83-1.70 (m, 2H), | Enantiomers | |
| | carbonyl)-1,2,4-oxadiazol- | | 1.66-1.40 (m, 2H). | had been | |
| | 5(4H)-one and the other is | | | separated at a | |
| | (R)-3-(8-(4-fluorophenyl)-6- | | | previous step | |
| | azaspiro[3.4]octane-6- | Enantiomer B (41b) | | | |
| | carbonyl)-1,2,4-oxadiazol- | 318 | ¹H NMR: (400 | Reverse-phase | N/A |
| | 5(4H)-one | [M + H]⁺ | MHz, DMSO-d₆, | flash | |
| | | | ppm) δ 13.04 (s, | chromatography: | |
| | | | 1H), 7.33-7.27 (m, | Column, C18 | |
| | | | 2H), 7.20-7.13 (m, | silica gel; Mobile | |
| | | | 2H), 4.13-3.97 (m, | Phase, 10% to | |
| | | | 1H), 3.90 (d, J = | 50% MeCN in | |
| | | | 7.2 Hz, 1H), 3.86- | water | |
| | | | 3.69 (m, 1H), 3.65 | (containing | |
| | | | (d, J = 4.4 Hz, 1H), | 0.1% HCl; | |
| | | | 3.37-3.29 (m, 1H), | detector, UV | |
| | | | 2.01-1.89 (m, 2H), | 254 nm | |
| | | | 1.83-1.68 (m, 2H), | Enantiomers | |
| | | | 1.66-1.54 (m, 1H), | had been | |
| | | | 1.54-1.40 (m, 1H). | separated at a | |
| | | | | previous step | |
| Example 42(b) and Example 42(a) | 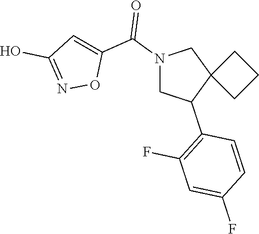 [SMILES: O=C(c1cc(O)no1)N1CC(c2ccc(F)cc2F)C2(CCC2)C1] | Enantiomer B (42b) | | | |
| | (8-(2,4-difluorophenyl)-6- | 335 | ¹H NMR (300 | Column: XA- | 8.1 |
| | azaspiro[3.4]octan-6-yl)(3- | [M + H]⁺ | MHz, DMSO-d₆, | CHIRAL ART | |
| | hydroxyisoxazol-5- | | ppm) δ 11.84 (br, | Cellulose-SB, | |
| | yl)methanone | | s, 1H), 7.47-7.21 | 3*25 cm, 5 um; | |
| | Enantiomer B | | (m, 2H), 7.11-7.06 | Mobile Phase: | |
| | (8-(2,4-difluorophenyl)-6- | | (m, 1H), 6.59 (s, | 15% EtOH | |
| | azaspiro[3.4]octan-6-yl)(3- | | 1H), 4.21-3.51 (m, | (containing | |
| | hydroxyisoxazol-5- | | 5H), 2.10-1.95 (m, | 0.1% FA)/ | |
| | yl)methanone | | 2H), 1.91-1.54 (m, | Hexane | |
| | Enantiomer A | | 4H). | (containing | |
| | One of Enantiomers A and B | | | 0.1% FA); Flow | |
| | is (S)-(8-(2,4-difluorophenyl)- | | | rate: 35 mL/min; | |
| | 6-azaspiro[3.4]octan-6-yl)(3- | | | Wavelength: | |
| | hydroxyisoxazol-5- | | | 254 nm | |
| | yl)methanone and the other is | Enantiomer A (42a) | | | |
| | (R)-(8-(2,4-difluorophenyl)-6- | 335 | ¹H NMR (300 | Column: XA- | 9.4 |
| | azaspiro[3.4]octan-6-yl)(3- | [M + H]⁺ | MHz, DMSO-d₆, | CHIRAL ART | |
| | hydroxyisoxazol-5- | | ppm) δ 11.84 (br, | Cellulose-SB, | |
| | yl)methanone | | s, 1H), 7.47-7.21 | 3*25 cm, 5 μm; | |
| | | | (m, 2H), 7.11-7.06 | Mobile Phase: | |
| | | | (m, 1H), 6.59 (s, | 15% EtOH | |
| | | | 1H), 4.21-3.51 (m, | (containing | |
| | | | 5H), 2.10-1.95 (m, | 0.1% FA)/ | |
| | | | 2H), 1.91-1.54 (m, | Hexane | |
| | | | 4H). | (containing | |
| | | | | 0.1% FA); Flow | |
| | | | | rate: 35 mL/min; | |
| | | | | Wavelength: | |
| | | | | 254 nm | |
| Example 43(a) and Example 43(b) | 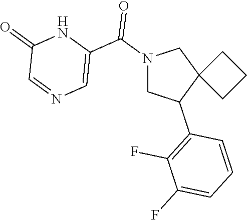 [SMILES: O=C(c1cncc(=O)[nH]1)N1CC(c2cccc(F)c2F)C2(CCC2)C1] | Enantiomer A 43(a) | | | |
| | 6-(8-(2,3-difluorophenyl)-6- | 346 | ¹H NMR: (400 | Column: XA- | 6.1 |
| | azaspiro[3.4]octane-6- | [M + H]⁺ | MHz, DMSO-d₆, | CHIRALPAK IG, | |
| | carbonyl)pyrazin-2(1H)-one | | ppm) δ 12.14 (br, | 3*25 cm, 5 μm; | |
| | Enantiomer A | | s, 1H), 8.35-8.19 | Mobile Phase: | |
| | 6-(8-(2,3-difluorophenyl)-6- | | (m, 1H), 8.15 (d, J = | 25% EtOH | |
| | azaspiro[3.4]octane-6- | | 11.1 Hz, 1H), | (containing | |
| | carbonyl)pyrazin-2(1H)-one | | 7.40-7.28 (m, 1H), | 0.1% FA)/3:1 | |
| | Enantiomer B | | 7.26-7.15 (m, 1H), | Hexane:DCM; | |
| | One of Enantiomers A and B | | 7.12-6.96 (m, 1H), | Flow rate: 35 | |
| | is (S)-6-(8-(2,3- | | 4.06-3.56 (m, 5H), | mL/min; | |
| | difluorophenyl)-6- | | 2.07-1.90 (m, 2H), | Wavelength: | |
| | azaspiro[3.4]octane-6- | | 1.87-1.55 (m, 4H). | 254 nm | |
| | carbonyl)pyrazin-2(1H)-one | Enantiomer B (43b) | | | |
| | and the other is (R)-6-(8-(2,3- | 346 | ¹H NMR: (400 | Column: XA- | 7.5 |
| | difluorophenyl)-6- | [M + H]⁺ | MHz, DMSO-d₆, | CHIRALPAK IG, | |
| | azaspiro[3.4]octane-6- | | ppm) δ 12.11 (br, | 3*25 cm, 5 μm; | |
| | carbonyl)pyrazin-2(1H)-one | | s, 1H), 8.35-8.19 | Mobile Phase: | |
| | | | (m, 1H), 8.15 (d, J = | 25% EtOH | |
| | | | 11.0 Hz, 1H), | (containing | |
| | | | 7.41-7.27 (m, 1H), | 0.1% FA)/3:1 | |
| | | | 7.27-7.15 (m, 1H), | Hexane:DCM; | |
| | | | 7.12-6.96 (m, 1H), | Flow rate: 35 | |
| | | | 4.06-3.58 (m, 5H), | mL/min; | |
| | | | 2.06-1.88 (m, 2H), | Wavelength: | |
| | | | 1.87-1.56 (m, 4H). | 254 nm | |
| Example 44(a) and Example 44(b) | 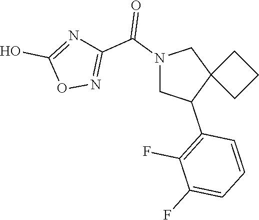 [SMILES: O=C(c1noc(O)n1)N1CC(c2cccc(F)c2F)C2(CCC2)C1] | Enantiomer A (44a) | | | |
| | 3-(8-(2,3-difluorophenyl)-6- | 336 | ¹H NMR: (400 | Reverse-phase | N/A |
| | azaspiro[3.4]octane-6- | [M + H]⁺ | MHz, DMSO-d₆, | flash | |
| | carbonyl)-1,2,4-oxadiazol- | | ppm) δ 13.06 (br, | chromatography | |
| | 5(4H)-one Enantiomer A | | s, 1H), 7.39-7.29 | Column, C18 | |
| | 3-(8-(2,3-difluorophenyl)-6- | | (m, 1H), 7.22-7.18 | silica gel; Mobile | |
| | azaspiro[3.4]octane-6- | | (m, 1H), 7.13-7.03 | Phase: 15% to | |
| | carbonyl)-1,2,4-oxadiazol- | | (m, 1H), 4.16-3.58 | 45% MeCN in | |
| | 5(4H)-one Enantiomer B | | (m, 5H), 2.05-1.95 | water | |
| | One of Enantiomers A and B | | (m, 2H), 1.88-1.55 | (containing | |
| | is (S)-3-(8-(2,3- | | (m, 4H). | 0.1% | |
| | difluorophenyl)-6- | | | NH₃•H₂O + 10 mm | |
| | azaspiro[3.4]octane-6- | | | ol/L NH₄HCO₃); | |
| | carbonyl)-1,2,4-oxadiazol- | | | Detector: UV | |
| | 5(4H)-one and the other is | | | 254 nm. | |
| | (R)-3-(8-(2,3-difluorophenyl)- | | | Enantiomers | |
| | 6-azaspiro[3.4]octane-6- | | | had been | |
| | carbonyl)-1,2,4-oxadiazol- | | | separated at a | |
| | 5(4H)-one | | | previous step | |
| | | Enantiomer B (44b) | | | |
| | | 336 | ¹H NMR: (400 | Reverse-phase | N/A |
| | | [M + H]⁺ | MHz, DMSO-d₆, | flash | |
| | | | ppm) δ 13.05 (br, | chromatography | |
| | | | s, 1H), 7.39-7.30 | Column, C18 | |
| | | | (m, 1H), 7.20 (q, J = | silica gel; Mobile | |
| | | | 7.7 Hz, 1H), | Phase: 10% to | |
| | | | 7.13-7.02 (m, 1H), | 50% MeCN in | |
| | | | 4.17-3.71 (m, 4H), | water | |
| | | | 3.70-3.59 (m, 1H), | (containing | |
| | | | 2.07-1.95 (m, 2H), | 0.1% | |
| | | | 1.88-1.56 (m, 4H). | NH₃•H₂O + 10 mm | |
| | | | | ol/L NH₄HCO₃); | |
| | | | | Detector: UV | |
| | | | | 254 nm | |
| | | | | Enantiomers | |
| | | | | had been | |
| | | | | separated at a | |
| | | | | previous step | |
| Example 45(a) and Example 45(b) | 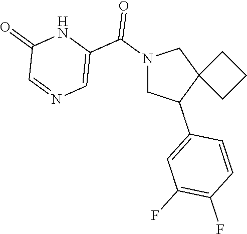 [SMILES: O=C(c1cncc(=O)[nH]1)N1CC(c2ccc(F)c(F)c2)C2(CCC2)C1] | Enantiomer A (45a) | | | |
| | 6-(8-(3,4-difluorophenyl)-6- | 346 | ¹H NMR: (300 | Column: XA | 3.0 |
| | azaspiro[3.4]octane-6- | [M + H]⁺ | MHz, DMSO-d₆, | CHIRALPAK IA, | |
| | carbonyl)pyrazin-2(1H)-one | | ppm) δ 12.13 (br, | 2*25 cm, 5 μm; | |
| | Enantiomer A | | s, 1H), 8.23 (br, s, | Mobile Phase: | |
| | 6-(8-(3,4-difluorophenyl)-6- | | 1H), 8.19-8.17 (m, | 30% EtOH | |
| | azaspiro[3.4]octane-6- | | 1H), 7.49-7.30 (m, | (containing | |
| | carbonyl)pyrazin-2(1H)-one | | 2H), 7.18-7.13 (m, | 0.1% FA)/3:1 | |
| | Enantiomer B | | 1H), 4.12-3.60 (m, | Hexane:DCM; | |
| | One of Enantiomers A and B | | 4H), 3.31 (s, 1H), | Flow rate: 25 | |
| | is (S)-6-(8-(3,4- | | 2.11-1.88 (m, 2H), | mL/min; | |
| | difluorophenyl)-6- | | 1.87-1.44 (m, 4H). | Wavelength: | |
| | azaspiro[3.4]octane-6- | | | 254 nm | |
| | carbonyl)pyrazin-2(1H)-one | | | | |
| | and the other is (R)-6-(8-(3,4- | | | | |
| | difluorophenyl)-6- | | | | |
| | azaspiro[3.4]octane-6- | | | | |
| | carbonyl)pyrazin-2(1H)-one | Enantiomer B (45b) | | | |
| | | 346 | ¹H NMR: (300 | Column: XA | 4.7 |
| | | [M + H]⁺ | MHz, DMSO-d₆, | CHIRALPAK IA, | |
| | | | ppm) δ 12.14 (br, | 2*25 cm, 5 μm; | |
| | | | s, 1H), 8.23 (br, s, | Mobile Phase: | |
| | | | 1H), 8.20-8.13 (m, | 30% EtOH | |
| | | | 1H), 7.51-7.29 (m, | (containing | |
| | | | 2H), 7.08 (s, 1H), | 0.1% FA)/3:1 | |
| | | | 4.12-3.60 (m, 4H), | Hexane:DCM; | |
| | | | 3.31 (s, 1H), 2.10- | Flow rate: 25 | |
| | | | 1.88 (m, 2H), | mL/min; | |
| | | | 1.87-1.44 (m, 4H). | Wavelength: | |
| | | | | 254 nm | |
| Example 46(b) and Example 46(a) | 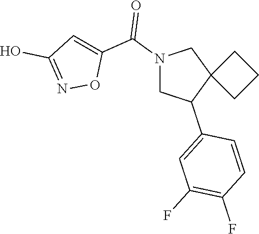 [SMILES: O=C(c1cc(O)no1)N1CC(c2ccc(F)c(F)c2)C2(CCC2)C1] | Enantiomer B (46b) | | | |
| | (8- | 335 | ¹H NMR: (300 | Column: XA- | 11.4 |
| | (3,4-difluorophenyl)-6- | [M + H]⁺ | MHz, DMSO-d₆, | CHIRAL ART | |
| | azaspiro[3.4]octan-6-yl)(3- | | ppm) δ 11.82 (br, | Cellulose-SC, | |
| | hydroxyisoxazol-5- | | s, 1H), 7.57-7.28 | 3*25 cm, 5 μm; | |
| | yl)methanone | | (m, 2H), 7.22-7.02 | Mobile Phase: | |
| | Enantiomer B | | (m, 1H), 6.59 (s, | 15% EtOH | |
| | (8-(3,4-difluorophenyl)-6- | | 1H), 4.10-3.92 (m, | (containing | |
| | azaspiro[3.4]octan-6-yl)(3- | | 1H), 3.86 (s, 1H), | 0.1% FA)/ | |
| | hydroxyisoxazol-5- | | 3.83-3.56 (m, 2H), | Hexane; Flow | |
| | yl)methanone | | 3.32-3.30 (m, 1H), | rate: 35 mL/min; | |
| | Enantiomer A | | 2.10-1.90 (m, 2H), | Wavelength: | |
| | One of Enantiomers A and B | | 1.88-1.58 (m, 3H), | 254 nm | |
| | is (S)-(8-(3,4-difluorophenyl)- | | 1.55-1.35 (m, 1H). | | |
| | 6-azaspiro[3.4]octan-6-yl)(3- | Enantiomer A (46a) | | | |
| | hydroxyisoxazol-5- | 335 | ¹H NMR: (300 | Column: XA- | 12.8 |
| | yl)methanone and the other is | [M + H]⁺ | MHz, DMSO-d₆, | CHIRAL ART | |
| | (R)-(8-(3,4-difluorophenyl)-6- | | ppm) δ 11.82 (br, | Cellulose-SC, | |
| | azaspiro[3.4]octan-6-yl)(3- | | s, 1H), 7.56-7.29 | 3*25 cm, 5 μm; | |
| | hydroxyisoxazol-5- | | (m, 2H), 7.22-7.02 | Mobile Phase: | |
| | yl)methanone | | (m, 1H), 6.58 (s, | 15% EtOH | |
| | | | 1H), 4.12-3.92 (m, | (containing | |
| | | | 1H), 3.86 (s, 1H), | 0.1% FA)/ | |
| | | | 3.84-3.55 (m, 2H), | Hexane; Flow | |
| | | | 3.32-3.30(m, 1H), | rate: 35 mL/min; | |
| | | | 2.09-1.88 (m, 2H), | Wavelength: | |
| | | | 1.88-1.57 (m, 3H), | 254 nm | |
| | | | 1.59-1.35 (m, 1H). | | |
| Example 47(b) and Example 47(a) | 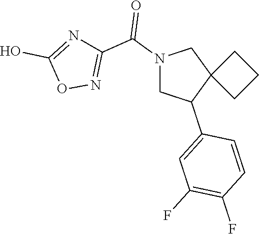 [SMILES: O=C(c1noc(O)n1)N1CC(c2ccc(F)c(F)c2)C2(CCC2)C1] | Enantiomer B (47b) | | | |
| | 3-(8-(3,4-difluorophenyl)-6- | 352 | ¹H NMR: (400 | Column: Sunfire | N/A |
| | azaspiro[3.4]octane-6- | [M + H]⁺ | MHz, DMSO-d₆, | Prep C18 OBD | |
| | carbonyl)-1,2,4-oxadiazol- | | ppm) δ 13.07 (br, | Column, | |
| | 5(4H)-one Enantiomer B | | s, 1H), 7.53-7.37 | 19*150 mm, | |
| | 3-(8-(3,4-difluorophenyl)-6- | | (m, 2H), 7.14 (s, | 5 μm; Mobile | |
| | azaspiro[3.4]octane-6- | | 1H), 4.17-4.00 (m, | Phase: 15% to | |
| | carbonyl)-1,2,4-oxadiazol- | | 1H), 3.98-3.88 (m, | 40% CAN/ | |
| | 5(4H)-one Enantiomer A | | 1H), 3.88-3.72 (m, | water | |
| | One of Enantiomers A and B | | 1H), 3.67 (s, 1H), | (containing | |
| | is (S)-3-(8-(3,4- | | 3.45-3.35 (m, 1H), | 0.05% HCl); | |
| | difluorophenyl)-6- | | 2.04-1.94 (m, 2H), | Flow rate: 30 | |
| | azaspiro[3.4]octane-6- | | 1.87-1.71 (m, 2H), | mL/min; | |
| | carbonyl)-1,2,4-oxadiazol- | | 1.69-1.59 (m, 1H), | Wavelength: | |
| | 5(4H)-one and the other is | | 1.59-1.43 (m, 1H). | 254 nm/220 nm | |
| | (R)- 3-(8-(3,4-difluorophenyl)- | | | Enantiomers | |
| | 6-azaspiro[3.4]octane-6- | | | had been | |
| | carbonyl)-1,2,4-oxadiazol- | | | separated at a | |
| | 5(4H)-one | | | previous step | |
| | | Enantiomer A (47a) | | | |
| | | 336 | ¹H NMR: (300 | Column: Sunfire | N/A |
| | | [M + H]⁺ | MHz, DMSO-d₆, | Prep C18 OBD | |
| | | | ppm) δ 13.07 (br, | Column, | |
| | | | s, 1H), 7.47-7.36 | 19*150 mm, | |
| | | | (m, 2H), 7.15 (s, | 5 μm; Mobile | |
| | | | 1H), 4.16-3.69 (m, | Phase: 15% to | |
| | | | 3H), 3.67 (s, 1H), | 40% CAN/ | |
| | | | 3.44-3.36 (m, 1H), | water | |
| | | | 2.05-1.92 (m, 2H), | (containing | |
| | | | 1.90-1.71 (m, 2H), | 0.05% HCl); | |
| | | | 1.70-1.41 (m, 2H). | Flow rate: 30 | |
| | | | | mL/min; | |
| | | | | Wavelength: | |
| | | | | 254 nm/220 nm | |
| | | | | Enantiomers | |
| | | | | had been | |
| | | | | separated at a | |
| | | | | previous step | |
| Example 48(b) and Example 48(a) | 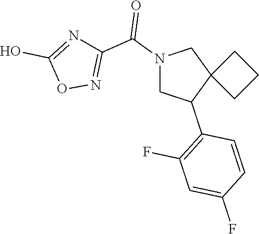 [SMILES: O=C(c1noc(O)n1)N1CC(c2ccc(F)cc2F)C2(CCC2)C1] | Enantiomer B (48b) | | | |
| | 3-(8-(2,4-difluorophenyl)-6- | 336 | ¹H NMR (400 | Column: XA- | 1.95 |
| | azaspiro[3.4]octane-6- | [M + H]⁺ | MHz, DMSO-d₆, | CHIRALPAK IF- | |
| | carbonyl)-1,2,4-oxadiazol- | | ppm) δ 13.08 (br, | 3, 50*4.6 mm, | |
| | 5(4H)-one Enantiomer B | | s, 1H), 7.47-7.26 | 3 um IF30CB- | |
| | 3-(8-(2,4-difluorophenyl)-6- | | (m, 2H), 7.10-7.01 | CP002; Mobile | |
| | azaspiro[3.4]octane-6- | | (m, 1H), 4.21-3.57 | Phase: 50% | |
| | carbonyl)-1,2,4-oxadiazol- | | (m, 5H), 2.12-1.91 | EtOH | |
| | 5(4H)-one Enantiomer A | | (m, 2H), 1.93-1.43 | (containing | |
| | One of Enantiomers A and B | | (m, 4H). | 0.5% FA)/ | |
| | is (S)-3-(8-(2,4- | | | MeOH; Flow | |
| | difluorophenyl)-6- | | | rate: 1 mL/min; | |
| | azaspiro[3.4]octane-6- | | | Wavelength: | |
| | carbonyl)-1,2,4-oxadiazol- | | | 254 nm | |
| | 5(4H)-one and the other is | Enantiomer A (48a) | | | |
| | (R)- 3-(8-(2,4-difluorophenyl)- | 336 | ¹H NMR (400 | Column: XA- | 2.52 |
| | 6-azaspiro[3.4]octane-6- | [M + H]⁺ | MHz, DMSO-d₆, | CHIRALPAK IF- | |
| | carbonyl)-1,2,4-oxadiazol- | | ppm) δ 13.05 (br, | 3, 50*4.6 mm, | |
| | 5(4H)-one | | s, 1H), 7.38-7.23 | 3 um IF30CB- | |
| | | | (m, 2H), 7.13-7.02 | CP002; Mobile | |
| | | | (m, 1H), 4.17-3.60 | Phase: 50% | |
| | | | (m, 5H), 2.06-1.90 | EtOH | |
| | | | (m, 2H), 1.85-1.56 | (containing | |
| | | | (m, 4H). | 0.5% FA)/ | |
| | | | | MeOH; Flow | |
| | | | | rate: 1 mL/min; | |
| | | | | Wavelength: | |
| | | | | 254 nm | |
| Example 49(a) and Example Ex 49(b) | 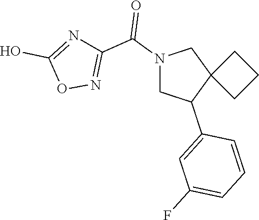 [SMILES: O=C(c1noc(O)n1)N1CC(c2cccc(F)c2)C2(CCC2)C1] | Enantiomer A (49a) | | | |
| | 3-(8-(3-fluorophenyl)-6- | 318 | ¹H NMR (300 | Column: XA- | 1.09 |
| | azaspiro[3.4]octane-6- | [M + H]⁺ | MHz, DMSO-d₆, | CHIRALPAK IA- | |
| | carbonyl)-1,2,4-oxadiazol- | | ppm) δ 13.09 (br, | 3, 50*4.6 mm, | |
| | 5(4H)-one Enantiomer A | | s, 1H), 7.61-7.27 | 3 um IA30CB- | |
| | 3-(8-(3-fluorophenyl)-6- | | (m, 1H), 7.27-6.99 | BX003; Mobile | |
| | azaspiro[3.4]octane-6- | | (m, 3H), 4.10-3.90 | Phase: 20% | |
| | carbonyl)-1,2,4-oxadiazol- | | (m, 1H), 3.89-3.83 | EtOH | |
| | 5(4H)-one Enantiomer B | | (m, 1H), 3.82-3.72 | (containing | |
| | One of Enantiomers A and B | | (m, 1H), 3.68-3.60 | 0.5% FA)/ | |
| | is (S)-3-(8-(3-fluorophenyl)-6- | | (m, 1H), 3.42-3.33 | MeCN; Flow | |
| | azaspiro[3.4]octane-6- | | (m, 1H), 2.07-1.93 | rate: 1 mL/min; | |
| | carbonyl)-1,2,4-oxadiazol- | | (m, 2H), 1.91-1.67 | Wavelength: | |
| | 5(4H)-one and the other is | | (m, 2H), 1.65-1.49 | 254 nm | |
| | (R)- 3-(8-(3-fluorophenyl)-6- | | (m, 2H). | | |
| | azaspiro[3.4]octane-6- | Enantiomer B (49b) | | | |
| | carbonyl)-1,2,4-oxadiazol- | 318 | ¹H NMR (300 | Column: XA- | 1.59 |
| | 5(4H)-one | [M + H]⁺ | MHz, DMSO-d₆, | CHIRALPAK IA- | |
| | | | ppm) δ 13.07 (br, | 3, 50*4.6 mm, | |
| | | | s, 1H), 7.48-7.34 | 3 um IA30CB- | |
| | | | (m, 1H), 7.23-7.03 | BX003; Mobile | |
| | | | (m, 3H), 4.16-3.91 | Phase: 20% | |
| | | | (m, 1H), 3.90-3.70 | EtOH | |
| | | | (m, 2H), 3.68-3.60 | (containing | |
| | | | (m, 1H), 3.40-3.36 | 0.5% FA)/ | |
| | | | (m, 1H), 2.06-1.92 | MeCN; Flow | |
| | | | (m, 2H), 1.90-1.69 | rate: 1 mL/min; | |
| | | | (m, 2H), 1.68-1.49 | Wavelength: | |
| | | | (m, 2H). | 254 nm | |

### BIOLOGICAL EXAMPLES

### Biological Example 1—mPTP Activity Assay in Isolated Rat Liver Mitochondria and Isolated Rat Brain Mitochondria

### Rat Liver Mitochondria Assay

Pharmacological inhibition or modulation of the mPTP can be measured in well characterised ‘Ca²⁺ retention’ assays performed in isolated mitochondria. In vitro, isolated mitochondria rapidly sequester exogenous Ca²⁺ until the intramitochondrial Ca²⁺ concentration reaches the threshold for mPTP activation. Once the pore is activated, mitochondrial integrity is compromised and the stored Ca²⁺ is released. The distribution of Ca²⁺ between extra- and intra-mitochondrial compartments can be measured in real time with the use of membrane-impermeant Ca²⁺ sensitive fluorescent dyes. Depending on the configuration of the assay, inhibition or modulation of the mPTP either delays the opening of the pore or increases the concentration of Ca²⁺ required to induce mPTP opening.

MPTP activity was measured in mitochondria freshly isolated from female Sprague Dawley (250 to 300 gram) rat livers using the following method. Cervical dislocation was performed on the rat. The liver was then perfused in-situ with ~40 ml cold Dulbecco's Phosphate Buffered Saline (DPBS) prior to dissection and transferred into 30 ml Isolation Buffer (250 mM Sucrose, 10 mM KCl, 1 mM EGTA, 1 mM EDTA, 25 mM HEPES, adjusted to pH 7.5 with 1M NaOH). Each lobe of the liver was then removed from the buffer, minced using tweezers and a scalpel into ~5 mm pieces then transferred into a 50 ml Potterton dounce homogenization tube on ice containing 30 ml ice-cold centrifugation buffer (300 mM Trehalose, 25 mM HEPES, 1 mM EGTA, 1 mM EDTA, 10 mM KCl, adjusted to pH 7.5 with 1M NaOH and supplemented with 0.1% bovine serum albumin (BSA) and complete protease inhibitor cocktail (one tablet of inhibitor per 50 mls of buffer). Homogenisation was carried out using a Teflon® pestle at 1800 rpm. The slurry was centrifuged at 800 g for 10 min at 4° C., then the supernatant centrifuged at 10,000 g for 10 min. The pellet was washed once with FLIPR assay buffer (75 mM Mannitol, 25 mM Sucrose, 5 mM Potassium Phosphate Monobasic, 20 mM Tris base, 100 mM KCl, 0.1% BSA adjusted to pH 7.4 with 5M HCl) centrifuged again, then resuspended in FLIPR assay buffer to a concentration of 8.8 mg/ml protein.

Tested compounds (10 mM stock in DMSO) were serially diluted in DMSO in half log steps to generate 10 test concentrations (final concentrations in assay 30 μM to 1 nM). An intermediate dilution of 5 μl DMSO samples into 247 μl FLIPR assay buffer was carried out prior to transfer of 5 μl into duplicate wells of a 384 well polypropylene assay plate. Control wells were 0.5% (v/v) DMSO and 5 μM cyclosporin A.

A stock mitochondria/Fluo5N assay solution was prepared in 5.6 ml FLIPR assay buffer (at RT) supplemented with succinate disodium salt (10 mM), rotenone (1 μM), Fluo5N pentapotassium salt (2 μM) and 1 ml mitochondria suspension, then transferred (15 μl) into the assay plate containing test compounds and incubated for 10 min at RT. Assay plates were processed for fluorescence detection on either a FLIPR Tetra (Molecular Devices) or CLARIOstar (BMG) plate reader. For kinetic fluorescence detection on the FLIPR, dye fluorescence was measured every 3 sec for a total of 10 min. After 12 sec, a 2.5 μl bolus of CaCl₂ (75 μM) was added from a source plate containing 675 μM CaCl₂ in FLIPR assay buffer. Alternatively, the 2.5 μl addition of CaCl₂ was performed using a Viaflow 384, plates were incubated at RT for 10 min, and dye fluorescence measured after 10 min on the CLARIOstar plate reader. pIC50 values for tested compounds were calculated using the fluorescence value collected at the 10 min timepoint on either plate reader, with % inhibition calculated using the DMSO control and cyclosporin A values as 0 and 100% respectively.

### Rat Brain Mitochondria Assay

MPTP activity was measured in brain mitochondria freshly isolated from female Sprague Dawley (250 to 300 gram) rats. Anaesthetised rats were perfused in-situ with ~40 ml cold Dulbecco's Phosphate Buffered Saline (DPBS), then brains dissected and transferred into 30 ml Isolation Buffer (225 mM mannitol, 75 mM sucrose, 1 mM EGTA, adjusted to pH 7.4 with 1M NaOH). The brain was minced using tweezers and a scalpel into ~5 mm pieces then transferred into a 50 ml Potterton Dounce homogenization tube on ice containing 10 ml ice-cold isolation buffer (as above with addition of Complete Protease inhibitor; 1 tablet per 50 ml buffer). Homogenisation was carried out using a Teflon® pestle at 1800 rpm. The slurry was centrifuged at 2000 g for 10 min at 4° C., then the supernatant centrifuged at 12,000 g for 9 min. The pellet was resuspended with a dounce homogeniser in isolation buffer as above but with the addition of 0.02% digitonin, centrifuged at 12,000 g for 11 min and finally resuspended in 5 ml modified isolation buffer (as above but with EGTA reduced to 0.1 mM).

Test compounds were prepared in 384 well polypropylene assay plates as described above for the liver mitochondria assay. A stock mitochondria/Fluo5N assay solution was prepared in 5.6 ml assay buffer (120 mM mannitol, 40 mM MOPS, 5 mM KH₂PO₄, 60 mM KCl, 10 mM pyruvate, 2 mM malate, 2 mM MgCl₂, 20 μM ADP, 1.26 μM oligomycin A, adjusted to pH 7.4) supplemented with Fluo5N pentapotassium salt (2 μM) and 1 ml mitochondria suspension, then transferred (15 μl) into the assay plate containing test compounds and incubated for 10 min at RT. Assay plates were then transferred to a FLIPR Tetra plate reader (Molecular Devices). Dye fluorescence was then measured every 3 sec for a total of 10 min. After 12 sec, a 2.5 μl bolus of Ca²⁺ (75 μM) was added from a source plate containing 675 μM CaCl₂ in FLIPR assay buffer. pIC50 values for test compounds were calculated using the fluorescence value collected at the 10 min timepoint with % inhibition calculated using the DMSO control and cyclosporin A values at 100% and 0% respectively.

General cytotoxicity was assessed using standard cell viability methods (Cell Titre Glo; Promega) in HEK293 and SHSY5Y cells, following incubation of test compound for between 24 and 96 hours.

Results: mPTP pIC₅₀ values for certain Example compounds of the invention in rat liver and/or brain mPTP assays are provided in Table 6 below. The results indicate that the tested compounds of the invention display inhibition of mPTP, with many Example compounds displaying pIC₅₀ values of 6.0 or greater. Example 35a showed the highest activity in the rat liver mitochondria assay. Example 36a showed the highest activity in the rat brain mitochondria assay. Table 6 also presents mPTP rat brain mitochondria pIC₅₀ values for certain Example compounds. These results indicate that the tested Example compounds are active against isolated rat liver mitochondria and isolated rat brain mitochondria.

TABLE 6
Summary of results from Biological Example 1

| Example No | mPTP Rat liver pIC50* | mPTP Rat brain pIC50 |
|---|---|---|
| 1a | 5.68 | 6.73 |
| 1b | 6.01 | 6.94 |
| 1c | <4.52 | NT |
| 2 | 5.80 | 6.90 |
| 3 | 5.14 | NT |
| 4 | 5.47 | 6.22 |
| 5a | 5.04 | NT |
| 5b | 5.33 | 6.55 |
| 5c | <4.52 | <4.52 |
| 6a | 6.39 | 7.34 |
| 6b | 4.59 | <4.52 |
| 7 | 5.95 | 7.11 |
| 8 | 6.04 | 6.97 |
| 9 | 5.28 | 6.61 |
| 10a | 6.18 | 7.03 |
| 10b | 5.19 | 6.07 |
| 11 | 4.75 | 6.25 |
| 12 | <4.52 | NT |
| 13 | 4.55 | NT |
| 14b | <4.52 | NT |
| 14a | 5.23 | 5.21 |
| 15a | 5.27 | 6.1 |
| 15b | <4.52 | 4.81 |
| 15d | <4.52 | <4.52 |
| 15c | 5.24 | 5.93 |
| 16a | 5.58 | 6.39 |
| 16b | <4.52 | <4.52 |
| 17a | 6 | 6.63 |
| 17b | <4.52 | <4.52 |
| 18 | 5.52 | 6.56 |
| 19a | 4.77 | 5.06 |
| 19b | <4.52 | <4.52 |
| 20b | <4.52 | <4.52 |
| 20a | <4.52 | 4.64 |
| 21a | 5.94 | 6.98 |
| 21b | <4.52 | <4.52 |
| 22 | 6.12 | 6.7 |
| 23a | 6.17 | 7.22 |
| 23b | <4.52 | <4.52 |
| 24 | 6.02 | 6.75 |
| 25a | 6.33 | 6.88 |
| 25b | <4.52 | 5.57 |
| 25c | 5.68 | 6.54 |
| 25d | <4.52 | <4.52 |
| 26a | 4.88 | 4.96 |
| 26b | <4.52 | <4.52 |
| 27b | <4.52 | <4.52 |
| 27a | 6.11 | 6.34 |
| 28a | 5.17 | 5.69 |
| 28b | <4.52 | <4.52 |
| 29a | 4.59 | 6.2 |
| 29b | <4.52 | <4.52 |
| 30a | 5.28 | 6.92 |
| 30b | <4.52 | <4.52 |
| 31a | 6.17 | 7.22 |
| 31b | 4.58 | <4.52 |
| 32a | 5.87 | 7.19 |
| 32b | <4.52 | <4.52 |
| 33a | 5.77 | 7.32 |
| 33b | <4.52 | <4.52 |
| 34a | 5.57 | 7.37 |
| 34b | <4.52 | <4.52 |
| 35a | 6.82 | 7.5 |
| 35b | 5.57 | 5.79 |
| 36b | <4.52 | <4.52 |
| 36a | 6.65 | 7.53 |
| 37a | 5.99 | 7.28 |
| 37b | <4.52 | <4.52 |
| 38b | <4.52 | <4.52 |
| 38a | 6.41 | 7.34 |
| 39b | <4.52 | <4.52 |
| 39a | 6.47 | 7.41 |
| 40b | 4.97 | <4.52 |
| 40a | 6.55 | 7.34 |
| 41a | 5.95 | 7.19 |
| 41b | <4.52 | <4.52 |
| 42b | <4.52 | <4.52 |
| 42a | 6.26 | 6.99 |
| 43a | 6.56 | 7.43 |
| 43b | 4.94 | 5.69 |
| 44a | 5.63 | 7.33 |
| 44b | <4.52 | 5.19 |
| 45a | 6.15 | 7.45 |
| 45b | <4.52 | 6.78 |
| 46b | <4.52 | 7.03 |
| 46a | 6.28 | 7.44 |
| 47b | <4.52 | <4.52 |
| 47a | 5.75 | 7 |
| 48b | <4.52 | <4.52 |
| 48a | 5.53 | 6.47 |
| 49a | 6.52 | NT |
| 49b | 4.52 | <4.52 |
| 50 | <4.57 | <4.52 |
| 51 | <4.57 | <4.52 |

NT—Not tested

Conclusion: The results of Biological Example 1 demonstrates that a significant proportion of tested compounds of the invention (or at least in the form of one stereoisomer thereof) are inhibitors of mPTP in rat liver and/or brain mPTP assays. A large number of the tested compounds are highly potent and have a pIC₅₀ value of 6.0 and above in the rat liver assay and/or a pIC₅₀ value of 7.0 and above in the rat brain assay. Therefore, the compounds of the invention are believed to be useful pharmaceuticals, particularly for the treatment or prophylaxis of diseases and disorders in which inhibition of mPTP provides a therapeutic or prophylactic effect.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### REFERENCES

Berge S et al. Pharmaceutical Salts. J. Pharm. Sci. 1977; 66; 1-19.
Greene et al. 4th Rev Ed., 2006, ISBN-10: 0471697540. “Protective Groups in Organic Synthesis”.
Yu et al., TDP-43 Triggers Mitochondrial DNA Release via mPTP to Activate cGAS/STING in ALS, Cell, Volume 183, Issue 3, 2020, pages 636-649.e18.
Jang et al., Proximal tubule cyclophilin D mediates kidney fibrogenesis in obstructive nephropathy, 2021, American Journal of Physiology: Renal physiology, 2021 321:4, F431-F442
Plyte et al., Cinnamic Anilides as New Mitochondrial Permeability Transition Pore Inhibitors Endowed with Ischemia-Reperfusion Injury Protective Effect in Vivo, J. Med Chem. 2014, 57, 5333-47
Chen et al., Probing Mitochondrial Permeability Transition Pore Activity in Nucleated Cells and Platelets by High-Throughput Screening Assays Suggests Involvement of Protein Phosphatase 2B in Mitochondrial Dynamics, Assay and Drug Development Technologies, 2018, 16, 445-45.

1. A compound according to formula (I): 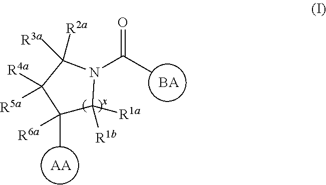
wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the carbon atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl, wherein said phenyl or phenyl fused to C₅₋₆cycloalkyl may be optionally substituted by one or more AA¹;
AA¹ is halo, C₂₋₆alkynyl, C₁₋₆alkyl C₁₋₆haloalkyl, C₁₋₆alkoxy, C₁₋₆haloalkoxy, C₃₋₆cycloalkyl, CN, OH, NR^{q}R^{r}, or NHSO₂R^{t};
R^{q} is H or C₁₋₄alkyl;
R^{r} is H or C₁₋₄alkyl;
R^{t} is C₁₋₄alkyl;
BA is a monocyclic or bicyclic heterocycle or a monocyclic or bicyclic heteroaryl, wherein said heterocycle or heteroaryl may be optionally substituted by one or more B^{1A}; and
B^{1A} is halo, C₁₋₆alkyl, C₁₋₆haloalkyl, oxo (=O), C₁₋₆alkoxy, C₁₋₆haloalkoxy, or C₀₋₆alkylene(OH);
or a salt and/or solvate thereof,
and provided that the compound of formula (I) is not 5-hydroxypyridin-3-yl)(8-phenyl-6-azaspiro[3.4]octan-6-yl)methanone:

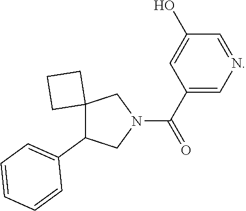 [SMILES: O=C(c1cncc(O)c1)N1CC(c2ccccc2)C2(CCC2)C1]

2. A compound of formula (IB) according to claim 1: 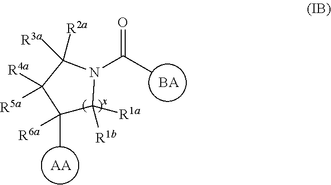
wherein:
R^{1a} is H or C₁₋₄alkyl;
R^{1b} is H or C₁₋₄alkyl;
R^{2a} is H, halo, C₁₋₄alkyl or C₁₋₄haloalkyl;
R^{3a} is H, halo or C₁₋₄alkyl;
R^{4a} is H or C₁₋₄alkyl;
R^{5a} is C₁₋₆alkyl, C₁₋₆haloalkyl, C₁₋₆alkoxy, C₁₋₆haloalkoxy, C₂₋₆alkenyl, C₂₋₆haloalkenyl, C₀₋₆alkylene(C₃₋₆cycloalkyl), C₀₋₆alkylene(OH); or R^{4a} and R^{5a} together with the atom to which they are attached form a C₃₋₆cycloalkyl wherein said cycloalkyl may be optionally substituted by one or more groups selected from C₁₋₄alkyl, C₁₋₄haloalkyl and halo;
R^{6a} is H, halo, C₁₋₄alkyl, C₁₋₄haloalkyl, C₁₋₄alkoxy or C₁₋₄haloalkoxy;
x is 0, 1 or 2;
AA is a phenyl or phenyl fused to C₅₋₆cycloalkyl optionally substituted by one or more AA¹;
AA¹ is halo, C₁₋₆alkyl, C₁₋₆haloalkyl, C₁₋₆alkoxy, C₁₋₆haloalkoxy, C₃₋₆cycloalkyl, CN, OH, NR^{q}R^{r}, or NHSO₂R^{t};
R^{q} is H or C₁₋₄alkyl;
R^{r} is H or C₁₋₄alkyl;
R^{t} is C₁₋₄alkyl;
BA is a monocyclic or bicyclic heterocycle or a monocyclic or bicyclic heteroaryl which may be optionally substituted by one or more B^{1A}; and
B^{1A} is halo, C₁₋₆alkyl, C₁₋₆haloalkyl, C₁₋₆alkoxy, C₁₋₆haloalkoxy, or C₀₋₆alkylene(OH);
or a salt and/or solvate thereof,
and provided that the compound of formula (IB) is not 5-hydroxypyridin-3-yl)(8-phenyl-6-azaspiro[3.4]octan-6-yl)methanone:

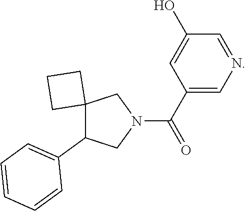 [SMILES: O=C(c1cncc(O)c1)N1CC(c2ccccc2)C2(CCC2)C1]

3. (canceled)

4. The compound or salt and/or solvate thereof according to claim 1, which is the compound or pharmaceutically acceptable salt and/or solvate thereof.

5. The compound or salt and/or solvate thereof according to claim 1, wherein R^{1a} and R^{1b} are each H.

6. The compound or salt and/or solvate thereof according to claim 1, wherein R^{2a} and R^{3a} are each H.

7. The compound or salt and/or solvate thereof according to claim 1, wherein R^{4a} and R^{5a} together with the atom to which they are attached form a C₃₋₆cycloalkyl, such as cyclobutyl.

8. The compound or salt and/or solvate thereof according to claim 1, wherein R^{4a} and R^{5a} are each methyl.

9. The compound or salt and/or solvate thereof according to claim 1, wherein R^{6a} is H.

10. The compound or salt and/or solvate thereof according to claim 1, wherein x is 1.

11. The compound or salt and/or solvate thereof according to claim 1, wherein AA is phenyl.

12. The compound or salt and/or solvate thereof according to claim 1, wherein AA is substituted by one or more AA¹, and wherein at least one AA¹ is halo.

13. (canceled)

14. A compound or salt and/or solvate thereof, which is selected from the group consisting of:
(6-hydroxypyrazin-2-yl)(8-phenyl-6-azaspiro[3.4]octan-6-yl)methanone;
(4-hydroxypyridin-2-yl)(8-phenyl-6-azaspiro[3.4]octan-6-yl)methanone;
(3,3-dimethyl-4-phenylpyrrolidin-1-yl)(5-hydroxypyridin-3-yl)methanone;
2-methyl-5-(8-phenyl-6-azaspiro[3.4]octane-6-carbonyl)-2,4-dihydro-3H-1,2,4-triazol-3-one;
(6-hydroxypyrazin-2-yl)(8-phenyl-6-azaspiro[3.4]octan-6-yl)methanone;
6-(8-(2-fluorophenyl)-6-azaspiro[3.4]octane-6-carbonyl)pyrazin-2(1H)-one;
6-(3,3-dimethyl-4-phenylpyrrolidine-1-carbonyl)pyrazin-2(1H)-one;
6-(3,3-dimethyl-4-phenylpiperidine-1-carbonyl)pyrazin-2(1H)-one;
6-(3,3-dimethyl-4-phenylpyrrolidine-1-carbonyl)pyrazin-2(1H)-one;
5-(8-(2-fluorophenyl)-6-azaspiro[3.4]octane-6-carbonyl)-2-methyl-2,4-dihydro-3H-1,2,4-triazol-3-one;
6-(3-methyl-4-phenylpyrrolidine-1-carbonyl)pyrazin-2(1H)-one;
8-Phenyl-6-(3H-1,2,3-triazole-4-carbonyl)-6-azaspiro[3.4]octane;
6-(3-ethyl-4-phenylpyrrolidine-1-carbonyl)pyrazin-2(1H)-one;
6-[8-(4-Chlorophenyl)-6-azaspiro[3.4]octane-6-carbonyl]-1H-pyrazin-2-one;
6-[4-Phenyl-2-azaspiro[4.4]nonane-2-carbonyl]-1H-pyrazin-2-one;
rac-2-{8-phenyl-6-azaspiro[3.4]octane-6-carbonyl}-3H-pyrimidin-4-one;
6-[(4-Ethynylphenyl)-6-azaspiro[3.4]octane-6-carbonyl]-1H-pyrazin-2-one;
4-[6-(6-oxo-1H-pyrazine-2-carbonyl)-6-azaspiro[3.4]octan-8-yl]benzonitrile;
2-[8-phenyl-6-azaspiro[3.4]octane-6-carbonyl]-3H-pyrimidin-4-one;
(3-hydroxyisoxazol-5-yl)(8-phenyl-6-azaspiro[3.4]octan-6-yl)methanone;
(3-hydroxyisoxazol-5-yl)(8-phenyl-6-azaspiro[3.4]octan-6-yl)methanone;
3-[8-Phenyl-6-azaspiro[3.4]octane-6-carbonyl]-4H-1,2,4-oxadiazol-5-one;
2-(2-fluoro-8-phenyl-6-azaspiro[3.4]octane-6-carbonyl)pyrimidin-4(3H)-one;
6-(8-phenyl-6-azaspiro[3.4]octane-6-carbonyl)pyridin-2(1H)-one;
6-(8-phenyl-6-azaspiro[3.4]octane-6-carbonyl)pyrimidin-2(1H)-one;
6-(8-(4-methoxyphenyl)-6-azaspiro[3.4]octane-6-carbonyl)pyrazin-2(1H)-one;
6-(8-(4-bromophenyl)-6-azaspiro[3.4]octane-6-carbonyl)pyrazin-2(1H)-one;
6-(8-(2-chlorophenyl)-6-azaspiro[3.4]octane-6-carbonyl)pyrazin-2(1H)-one;
6-(8-(3-chlorophenyl)-6-azaspiro[3.4]octane-6-carbonyl)pyrazin-2(1H)-one;
6-8-(5-chloro-2-fluorophenyl)-6-azaspiro[3.4]octane-6-carbonyl)pyrazin-2(1H)-one;
6-(8-(3-chloro-2-fluorophenyl)-6-azaspiro[3.4]octane-6-carbonyl)pyrazin-2(1H)-one;
3-(8-phenyl-6-azaspiro[3.4]octane-6-carbonyl)-1,2,4-oxadiazol-5(4H)-one;
6-(8-(4-fluorophenyl)-6-azaspiro[3.4]octane-6-carbonyl)pyrazin-2(1H)-one;
(8-(4-fluorophenyl)-6-azaspiro[3.4]octan-6-yl)(3-hydroxyisoxazol-5-yl)methanone;
6-(8-(2,4-difluorophenyl)-6-azaspiro[3.4]octane-6-carbonyl)pyrazin-2(1H)-one;
(8-(3-fluorophenyl)-6-azaspiro[3.4]octan-6-yl)(3-hydroxyisoxazol-5-yl)methanone;
(8-(2,3-difluorophenyl)-6-azaspiro[3.4]octan-6-yl)(3-hydroxyisoxazol-5-yl)methanone;
6-(8-(3-fluorophenyl)-6-azaspiro[3.4]octane-6-carbonyl)pyrazin-2(1H)-one;
3-(8-(4-fluorophenyl)-6-azaspiro[3.4]octane-6-carbonyl)-1,2,4-oxadiazol-5(4H)-one;
(8-(2,4-difluorophenyl)-6-azaspiro[3.4]octan-6-yl)(3-hydroxyisoxazol-5-yl)methanone;
6-(8-(2,3-difluorophenyl)-6-azaspiro[3.4]octane-6-carbonyl)pyrazin-2(1H)-one;
3-(8-(2,3-difluorophenyl)-6-azaspiro[3.4]octane-6-carbonyl)-1,2,4-oxadiazol-5(4H)-one 6-(8-(3,4-difluorophenyl)-6-azaspiro[3.4]octane-6-carbonyl)pyrazin-2(1H)-one;
(8-(3,4-difluorophenyl)-6-azaspiro[3.4]octan-6-yl)(3-hydroxyisoxazol-5-yl)methanone;
3-(8-(3,4-difluorophenyl)-6-azaspiro[3.4]octane-6-carbonyl)-1,2,4-oxadiazol-5(4H)-one;
3-(8-(2,4-difluorophenyl)-6-azaspiro[3.4]octane-6-carbonyl)-1,2,4-oxadiazol-5(4H)-one; and
3-(8-(3-fluorophenyl)-6-azaspiro[3.4]octane-6-carbonyl)-1,2,4-oxadiazol-5(4H)-one;
or a salt and/or solvate of any one thereof.

15. The compound or salt and/or solvate thereof according to claim 1, which is a compound of formula (I).

16. The compound or salt and/or solvate thereof according to claim 1, which is salt of a compound of formula (I).

17. (canceled)

18. A pharmaceutical composition comprising a compound of formula (I) or a pharmaceutically acceptable salt and/or solvate thereof according to claim 4, and a pharmaceutically acceptable carrier or excipient.

19-21. (canceled)

22. A method of treating a disease or disorder in which inhibition of mPTP provides a therapeutic effect, wherein the proviso of formula (I) does not apply, which comprises administrating to a human subject in need thereof an effective amount of a compound or pharmaceutically acceptable salt and/or solvate thereof according to claim 4.

23. The method according to claim 22, wherein the disease or disorder is selected from the group consisting of: Parkinson's disease, dementia with Lewy bodies, Alzheimer's disease, amyotrophic lateral sclerosis, multiple sclerosis, frontal temporal dementia, chemotherapy induced neuropathy, Huntington's disease, spinocerebellar ataxias, progressive supranuclear palsy, hereditary spastic paraplegia, Duchenne muscular dystrophy, congenital muscular dystrophy, traumatic brain injury, Friedreich's ataxia, AIDS dementia complex, depressive disorders, schizophrenia, epilepsy, acute myocardial infarction, stroke, kidney ischemia reperfusion injury, organ damage during transplantation, hepatic steatosis, diabetes, diabetic retinopathy, cognitive decline, obesity and feeding behaviours, non-alcoholic fatty liver disease, acute pancreatitis, systemic lupus, organ failure in sepsis, hepatitis, bone weakness in aging in osteoporosis, sarcopenia, chronic kidney disease associated with APOL1 genetic variants, chronic kidney disease, mitochondria Reye syndrome, Leber's hereditary optic neuropathy and associated disorders, Facial onset sensory and motor neuronopathy, Primary lateral sclerosis, Progressive muscular atrophy, Inclusion body myopathy associated with early-onset Paget disease of the bone and Frontotemporal lobar degeneration dementia, Perry disease, Chronic traumatic encephalopathy, Hippocampal sclerosis dementia, Limbic-predominant age-related TDP-43 encephalopathy, and Cerebral age-related TDP-43 with sclerosis.

24-34. (canceled)

35. The compound or salt and/or solvate thereof according to claim 1, wherein BA is selected from the group consisting of: 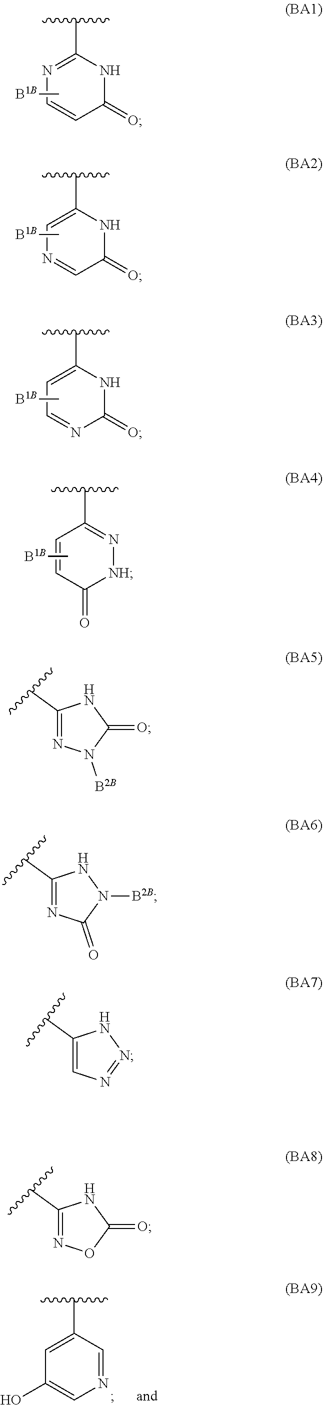 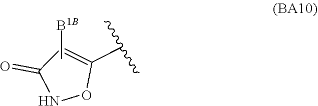
wherein:
B^{1B} is H or C₁₋₆alkyl, such as methyl; and
B^{2B} is C₁₋₆alkyl, such as methyl.

36. The compound or salt and/or solvate thereof according to claim 35, wherein BA is selected from the group consisting of: 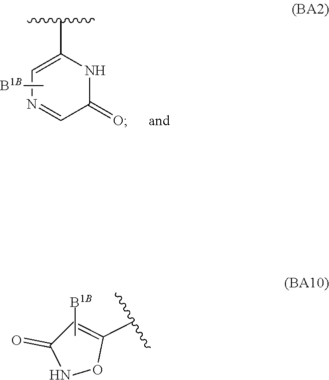
wherein:
B^{1B} is H or methyl.
